(19) United States
(12) Patent Application Publication
(10) Pub. No.: US 2024/0390400 A1
(43) Pub. Date: Nov. 28, 2024

(54) ENPP1 INHIBITORS AS INHIBITORS OF METASTASIS

(71) Applicant: Angarus Therapeutics, Inc., Portola Valley, CA (US)

(72) Inventors: Jackie Papkoff, San Francisco, CA (US); Randolph Mellus Johnson, Half Moon Bay, CA (US); Neil A. Verity, Helotes, TX (US); Nigel Ray, Portola Valley, CA (US); James Johnston, Westlake Village, CA (US)

(21) Appl. No.: 18/647,254

(22) Filed: Apr. 26, 2024

Related U.S. Application Data
(63) Continuation of application No. PCT/US2022/078900, filed on Oct. 28, 2022.
(60) Provisional application No. 63/273,702, filed on Oct. 29, 2021.

(51) Int. Cl. A61K 31/675; A61K 31/704; A61K 39/395; A61P 35/04

(52) U.S. Cl. CPC A61K 31/675; A61K 31/704; A61K 39/3955; A61P 35/04

(57) ABSTRACT

Methods are provided for the treatment or prevention of metastasis by the inhibition of ENPP1. Aspects of the subject methods include contacting a sample with a cell impermeable ENPP1 inhibitor to treat or prevent metastasis. Aspects of the methods include administering to a subject in need thereof a therapeutically effective amount of an immune checkpoint inhibitor, a chemotherapeutic agent, and/or radiation treatment, in combination with a therapeutically effective amount of a cell impermeable ENPP1 inhibitor to treat or prevent metastasis.

### CROSS REFERENCE TO RELATED APPLICATIONS

This application is a bypass continuation of PCT/US2022/078900, filed on Oct. 28, 2022, which claims priority to and the benefit of U.S. Provisional Patent Application No. 63/273,702, filed Oct. 29, 2021, the contents of which are incorporated by reference in their entirety herein.

### INTRODUCTION

Cyclic guanosine monophosphate-adenosine monophosphate (cGAMP) activates the Stimulator of Interferon Genes (STING) pathway, which is an important anti-cancer innate immune pathway. The cGAS-cGAMP-STING pathway gets activated in presence of cytoplasmic DNA either due to microbial infection or patho-physiological condition, including cancer and autoimmune disorder. Cyclic GMP-AMP synthase (cGAS) belongs to the nucleotidyltransferase family and is a universal DNA sensor that is activated upon binding to cytosolic dsDNA to produce the signaling molecule (2′-5′, 3′-5′) cyclic GMP-AMP (or 2′, 3′-cGAMP or cyclic guanosine monophosphate-adenosine monophosphate, cGAMP). Acting as a second messenger during microbial infection, 2′, 3′-cGAMP binds and activates STING, leading to production of type I interferon (IFN) and other co-stimulatory molecules that trigger the immune response. Besides its role in infectious disease, the STING pathway has is under exploration as a target for cancer immunotherapy and autoimmune diseases.

Ectonucleotide pyrophosphatase/phosphodiesterase 1 (ENPP1) is the dominant hydrolase of cGAMP that can degrade cGAMP. ENPP1 is a member of the ecto-nucleotide pyrophosphatase/phosphodiesterase (ENPP) family. The encoded protein is a type II transmembrane glycoprotein comprising two identical disulfide-bonded subunits. The ENPP1 protein has broad specificity and can cleave a variety of substrates, including phosphodiester bonds of nucleotides and nucleotide sugars and pyrophosphate bonds of nucleotides and nucleotide sugars. This protein may function to hydrolyze nucleoside 5′ triphosphates to their corresponding monophosphates and may also hydrolyze diadenosine polyphosphates.

Metastasis is a major cause of death among patients with cancer. Despite progress that has been achieved with therapeutic approaches, a complete cure awaits more effective strategies.

The process of metastasis involves multiple steps that include release of cancer cells from the primary site, intravasation to neighboring vessels, transport to the site of metastasis through blood flow, extravasation and/or infarction to the distant organ, and re-growth of the invading cells with acquisition of nutrition in the new environment. Therefore multiple genes are expected to be associated with the process of metastasis. Although many investigators have been working on this clinically important issue, the precise mechanisms or identification of the critical genes remain to be clarified.

Thus, there is need to prevent or reduce metastasis in cancer patients.

### SUMMARY

Methods are provided for treating, reducing, or preventing or reducing metastasis of cancer in a subject in need thereof, the methods comprising administering to the subject a therapeutically effective amount of a composition comprising an ectonucleotide pyrophosphatase/phosphodiesterase 1 (ENPP1) inhibitor.

In some aspects, the method further comprises administering to the subject a therapeutically effective amount of a composition comprising an immune checkpoint inhibitor in combination with the composition comprising an ectonucleotide pyrophosphatase/phosphodiesterase 1 (ENPP1) inhibitor. In some embodiments, the immune checkpoint inhibitor is a cytotoxic T-lymphocyte-associated antigen 4 (CTLA-4) inhibitor, a programmed death 1 (PD-1) inhibitor, or a PD-L1 inhibitor. In some embodiments, the immune check point inhibitor is atezolizumab, avelumab, durvalumab, cemiplimab, ipilimumab, pembrolizumab, or nivolumab. In certain embodiments, the immune check point inhibitor is ipilimumab, pembrolizumab, or nivolumab.

In some aspects, the method further comprises administering to the subject a therapeutically effective amount of a composition comprising a chemotherapeutic agent in combination with the composition comprising an ectonucleotide pyrophosphatase/phosphodiesterase 1 (ENPP1) inhibitor. In some embodiments, the chemotherapeutic agent is a cytotoxic agent. In some embodiments, the chemotherapeutic agent is selected from the group consisting of daunorubicin, doxorubicin, epirubicin, idarubicin, mitoxantrone, and valrubicin. In certain embodiment, the chemotherapeutic agent is doxorubicin.

In some aspects, the method further comprises administering radiation therapy to the subject.

Provided for the methods herein is an ectonucleotide pyrophosphatase/phosphodiesterase 1 (ENPP1) inhibitor, or a composition comprising said inhibitor.

In some aspects, the ENPP1 inhibitor comprises the formula (VI):

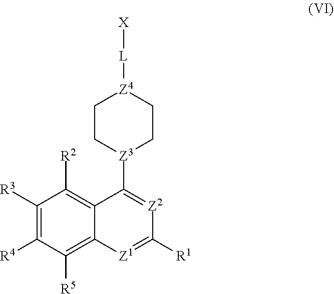

wherein, X is a hydrophilic head group selected from boronic acid, hydroxamic acid, phosphonic acid, phosphonate, phosphonate ester, phosphate, phosphate ester, thiophosphate, thiophosphate ester, phosphoramidate and thiophosphoramidate; L is a linker; Z¹ and Z² are each independently selected from CR¹ and N; Z³ and Z⁴ are each independently selected from CR and N, wherein R is H, alkyl or substituted alkyl; each R¹ is independently selected from H, cyano, trifluoromethyl, halogen, amino, alkyl, substituted alkyl, alkenyl, substituted alkenyl, heterocycle and substituted heterocycle; R² and R⁵ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle; R³ and R⁴ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle; or R³ and R⁴ together with the carbon atoms to which they are attached form a fused ring selected from heterocycle, substituted heterocycle, cycloalkyl, substituted cycloalkyl, aryl and substituted aryl; or a pro-drug, a pharmaceutically acceptable salt or a solvate thereof.

In some aspects, L is selected from —CH₂—, —(CH₂)₂—, —(CH₂)₃—, —(CH₂)₄—, —(CH₂)₅— and —(CH₂)₆—; X is selected from:

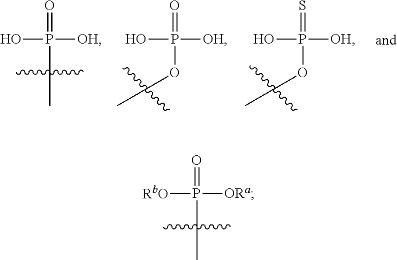

wherein: R^{a} and R^{b} are each independently selected from aryl, alkyl, and —CH₂OC(O)R^{c}, —CH₂OC(O)OR^{e}; and wherein R^{e} is alkyl.

In some aspects, the ENPP1 inhibitor is of the formula:

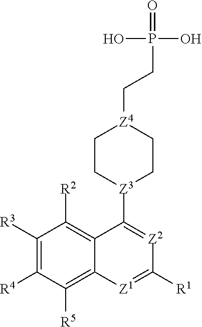

wherein, Z¹ and Z² are each N; Z³ is N; and Z⁴ is CH or N.

In some aspects, the portion of the ENPP1 inhibitor represented by

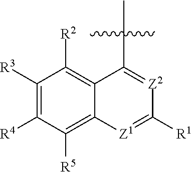

is selected from:

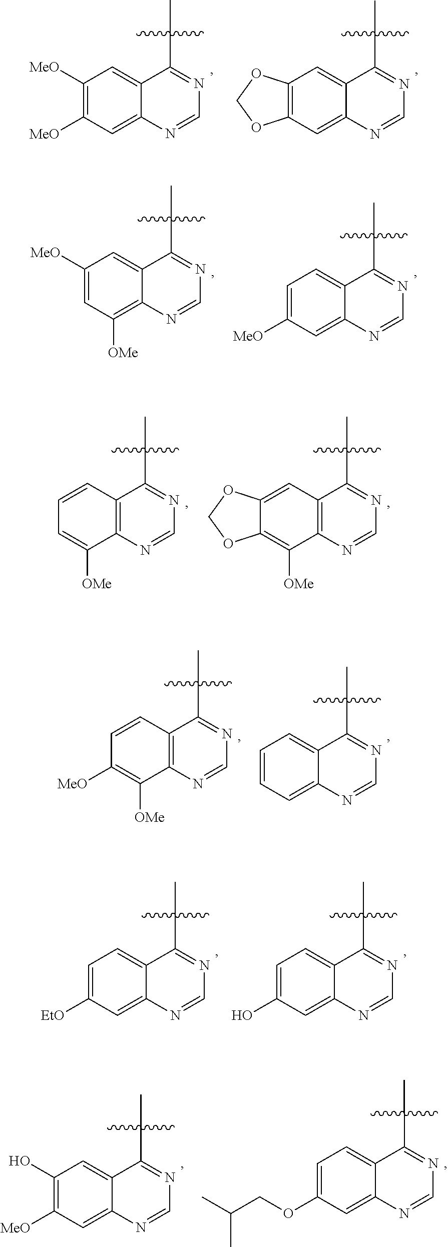

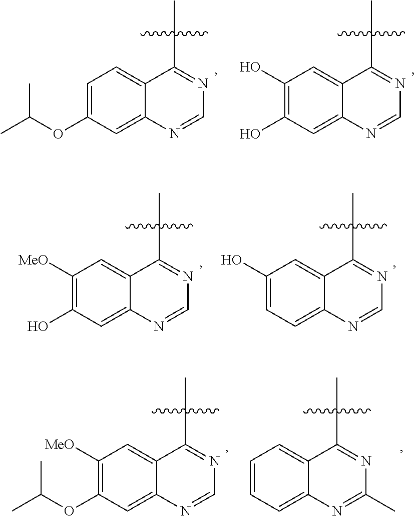

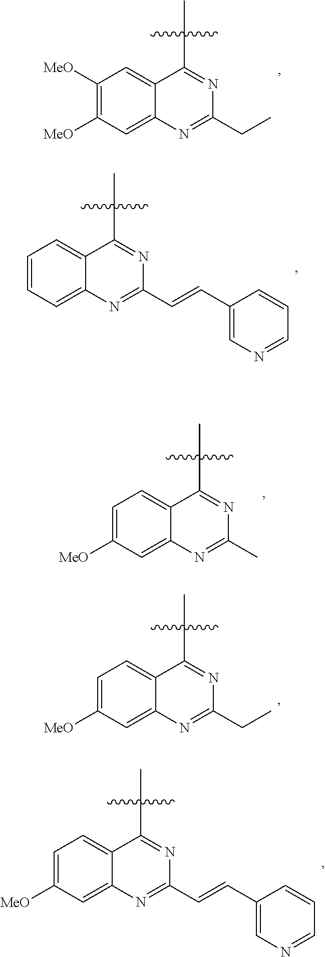 [SMILES: *c1nc(CC)nc2cc(OC)c(OC)cc12 ; *c1nc(/C=C/c2cccnc2)nc2ccccc12 ; *c1nc(C)nc2cc(OC)ccc12 ; *c1nc(CC)nc2cc(OC)ccc12 ; *c1nc(/C=C/c2cccnc2)nc2cc(OC)ccc12]

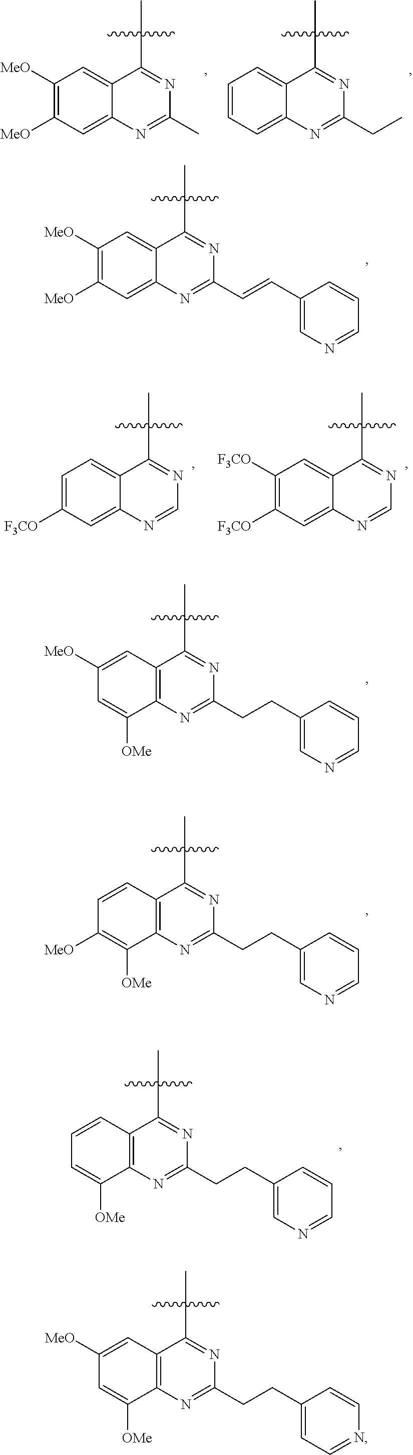

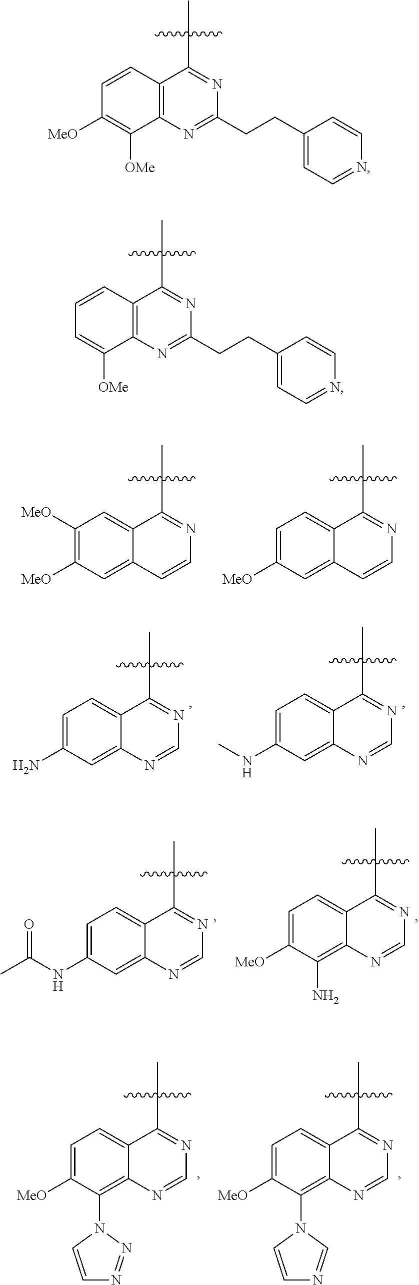

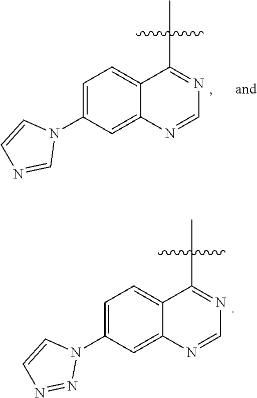 [SMILES: *c1ncnc2cc(-n3ccnc3)ccc12 ; *c1ncnc2cc(-n3ccnn3)ccc12]

In some aspects, the ENPP1 inhibitor is selected from the group consisting of

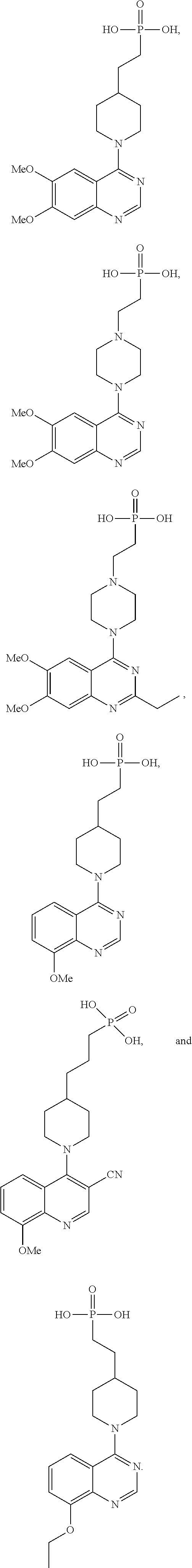

In some aspects, the inhibitor is a compound of Table 1 or Table 2.

In some aspects, the composition further comprises an additional adjuvant, optionally wherein the additional adjuvant is selected from the group consisting of: alum, CpG oligonucleotides, Freund's adjuvant, 1018 ISS, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, lipopolysacharride, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon, mycobacterial extracts, synthetic bacterial cell wall mimics, and Ribi's Detox.

Also provided for herein is a method of treating, reducing, or preventing metastasis in a subject, for treating, reducing, or preventing metastasis of cancer in a subject in need thereof, the methods comprising administering to the subject a therapeutically effective amount of a composition comprising an ectonucleotide pyrophosphatase/phosphodiesterase 1 (ENPP1) inhibitor, the method further comprising administering an immune checkpoint inhibitor and radiation treatment. Administration of the ENPP1 inhibitor, immune checkpoint inhibitor, and/or radiation can be simultaneous or sequential. Sequential administration can be after several minutes, hours, or days.

In some embodiments of the methods provided herein, the subject is to undergo or has undergone resection of a malignant tumor. In some embodiments of the methods provided herein, the method comprises suppressing or preventing the recurrence of the cancer. In some embodiments of the methods provided herein, the method comprises suppressing or preventing malignant tumor cells from colonizing or invading vascular tissue.

Also provided for use in the methods herein is a pharmaceutical composition comprising: a) an ectonucleotide pyrophosphatase/phosphodiesterase 1 (ENPP1) inhibitor; and b) a nanoparticle, wherein the pharmaceutical composition is formulated for mucosal delivery.

In some aspects, the nanoparticle comprises a liposome. In some aspects, the nanoparticle comprises a hydrogel. In some aspects, the liposome comprises a pulmonary surfactant, a pulmonary surfactant membrane constituent, and/or a pulmonary surfactant biomimetic. In some aspects, the liposome, the pulmonary surfactant, the pulmonary surfactant membrane constituent, and/or the pulmonary surfactant biomimetic is negatively charged.

In some aspects, the mucosal delivery comprises buccal delivery, sublingual delivery, or intranasal delivery.

These and other advantages and features of the disclosure will become apparent to those persons skilled in the art upon reading the details of the compositions and methods of use, which are more fully described below.

### BRIEF DESCRIPTION OF THE FIGURES

The invention is best understood from the following detailed description when read in conjunction with the accompanying figures. The patent or application file contains at least one figure executed in color. It is emphasized that, according to common practice, the various features of the figures are not to-scale. On the contrary, the dimensions of the various features are arbitrarily expanded or reduced for clarity. Included in the drawings are the following figures. It is understood that the figures, described below, are for illustration purposes only. The figures are not intended to limit the scope of the present teachings in any way.

FIG. 1 shows a plot of EMT-6 tumor volume in mm³ over time for tumors treated with irradiation (IR), compound 76, and an anti-PDL1 compound, alone and in combination.

FIG. 2 shows a plot of EMT-6 tumor volume in mm³ over time for tumors treated with irradiation (IR), compound 76, and an anti-PDL1 compound, alone and in combination.

FIG. 3A-FIG. 3E show plots of EMT-6 tumor volume in mm³ over time for tumors treated with irradiation (IR), compound 76, and an anti-PDL1 compound, alone and in combination.

FIG. 4 and FIG. 5 show images of metastasis for EMT-6 lung tumors treated with irradiation (IR), compound 76, and an anti-PDL1 compound, alone and in combination.

FIG. 6 shows data of the number and percentage of animals with metastasis for EMT-6 lung tumors treated with irradiation (IR), compound 76, and an anti-PDL1 compound, alone and in combination.

FIG. 7 shows a plot of tumor size and tumor area percent for EMT-6 lung tumors treated with irradiation (IR), compound 76, and an anti-PDL1 compound, alone and in combination.

FIG. 8 shows a plot of percent of animals with metastasis of EMT-6 lung tumors treated with irradiation (IR), compound 76, and an anti-PDL1 compound, alone and in combination.

FIG. 9 is a plot of primary tumor volume in mm³ over time for 4T1 tumor-bearing mice treated with vehicle, AG-3132, doxorubicin, or AG-3132+doxorubicin at day 0 to day 21, with associated P-values for AG-3132, doxorubicin, and AG-3132+doxorubicin groups compared to the vehicle group.

FIG. 10A is a histogram comparing the mean primary tumor volume in mm³ over time for 4T1 tumor-bearing mice treated with vehicle, AG-3132, doxorubicin, or AG-3132+doxorubicin.

FIG. 10B is a dot plot comparing the primary tumor volume in 4T1 tumor-bearing mice treated with vehicle, AG-3132, doxorubicin, or AG-3132+doxorubicin.

FIG. 11 is a plot of body weight over time for 4T1 tumor-bearing mice treated with vehicle, AG-3132, doxorubicin, or AG-3132+doxorubicin.

FIG. 12A is a histogram comparing the extent of lymph node metastasis (measured by mean luciferase activity) in 4T1 tumor-bearing mice treated with vehicle, AG-3132, doxorubicin, or AG-3132+doxorubicin.

FIG. 12B is a dot plot comparing the extent of lymph node metastasis (measured by in vivo luciferase activity) in 4T1 tumor-bearing mice treated with vehicle, AG-3132, doxorubicin, or AG-3132+doxorubicin.

FIG. 13A is a histogram comparing the extent of lumbar spine metastasis (measured by mean luciferase activity) in 4T1 tumor-bearing mice treated with vehicle, AG-3132, doxorubicin, or AG-3132+doxorubicin.

FIG. 13B is a dot plot comparing the extent of lumbar spine metastasis (measured by in vivo luciferase activity) in 4T1 tumor-bearing mice treated with vehicle, AG-3132, doxorubicin, or AG-3132+doxorubicin.

FIG. 14A is a histogram comparing the extent of lung metastasis (measured by mean luciferase activity) in 4T1 tumor-bearing mice treated with vehicle, AG-3132, doxorubicin, or AG-3132+doxorubicin.

FIG. 14B is a dot plot comparing the extent of lung metastasis (measured by in vivo luciferase activity) in 4T1 tumor-bearing mice treated with vehicle, AG-3132, doxorubicin, or AG-3132+doxorubicin.

FIG. 15A is a histogram comparing the extent of ileum metastasis (measured by mean luciferase activity) in 4T1 tumor-bearing mice treated with vehicle, AG-3132, doxorubicin, or AG-3132+doxorubicin.

FIG. 15B is a dot plot comparing the extent of ileum metastasis (measured by in vivo luciferase activity) in 4T1 tumor-bearing mice treated with vehicle, AG-3132, doxorubicin, or AG-3132+doxorubicin.

### DETAILED DESCRIPTION

As summarized above, aspects of the present disclosure include methods for treating, reducing, or preventing metastasis of cancer in a subject in need thereof, the methods comprising administering to the subject a therapeutically effective amount of a composition comprising an ectonucleotide pyrophosphatase/phosphodiesterase 1 (ENPP1) inhibitor.

In some aspects, the method further comprises administering to the subject a therapeutically effective amount of a composition comprising an immune checkpoint inhibitor in combination with the composition comprising an ectonucleotide pyrophosphatase/phosphodiesterase 1 (ENPP1) inhibitor.

In some aspects, the method further comprises administering radiation therapy to the subject.

### ENPP1-Inhibitor Compounds

As summarized above, aspects of the disclosure include methods of used of ENPP1 inhibitor compounds. The subject compounds can include a core structure based on an aryl or heteroaryl ring system, e.g., a quinazoline, isoquinoline or pyrimidine group, which is linked to a hydrophilic head group. The linker between the aryl or heteroaryl ring system and the hydrophilic head group can include a monocyclic carbocycle or heterocycle and an acyclic linker. In some cases, the linker includes a 1,4-disubstituted 6-membered ring, such as cyclohexyl, piperidinyl or piperazinyl. The aryl or heteroaryl ring system is optionally further substituted. Exemplary ENPP1 inhibitor compounds of interest including quinazoline, isoquinoline and pyrimidine ring systems are set forth in formulae I IV, V, VI and VII and the following structures 1-106, 201-222 and 226.

In some cases, the subject ENPP1 inhibitor compound is of formula (I):

Y-A-L-X (I)

wherein:
Y is selected from aryl, substituted aryl, heteroaryl, substituted heteroaryl, carbocycle, substituted carbocycle, heterocycle and substituted heterocycle;
A is selected from carbocycle, substituted carbocycle, heterocycle and substituted heterocycle;
L is a covalent bond or a linker; and
X is a hydrophilic head group,
or a pro-drug, a pharmaceutically acceptable salt or a solvate thereof.

The term “hydrophilic head group” refers to a linked group of the subject compounds that is hydrophilic and well solvated in aqueous environments e.g., under physiological conditions, and has low permeability to cell membranes. In some cases, by low permeability to cell membranes is meant a permeability coefficient of 10⁻⁴ cm/s or less, such as 10⁻⁵ cm/s or less, 10⁻⁶ cm/s or less, 10⁻⁷ cm/s or less, 10⁻⁸ cm/s or less, 10⁻⁹ cm/s or less, or even less, as measured via any convenient methods of passive diffusion for an isolated hydrophilic head group through a membrane (e.g., cell monolayers such as the colorectal Caco-2 or renal MDCK cell lines). See e.g., Yang and Hinner, Methods Mol Biol. 2015; 1266: 29-53.

The hydrophilic head group can impart improved water solubility and reduced cell permeability upon the molecule to which it is attached. The hydrophilic head group may be any convenient hydrophilic group that is well solvated in aqueous environments and which has low permeability to membranes. In certain instances, the hydrophilic group is a discrete functional group (e.g., as described herein) or a substituted version thereof. In general terms, larger, uncharged polar groups or charged groups have low permeability. In some cases, the hydrophilic head group is charged, e.g., positively or negatively charged. In some embodiments, the hydrophilic head group is not cell permeable and imparts cell impermeability upon the subject compound. It is understood that a hydrophilic headgroup, or a prodrug form thereof, can be selected to provide for a desired cell permeability of the subject compound. In certain cases, the hydrophilic head group is a neutral hydrophilic group. In some cases, the hydrophilic head group comprises a promoiety. In certain instances, the subject compound is cell permeable.

In some embodiments of formula (I), the hydrophilic head group (X) is selected from boronic acid, hydroxamic acid, phosphonic acid or phosphonate, phosphonate ester, phosphate, phosphate ester, thiophosphate, thiophosphate ester, phosphoramidate, thiophosphoramidate, sulfonate, sulfonic acid, sulfate, hydroxamic acid, keto acid, amide and carboxylic acid. In some embodiments of formula (I), the hydrophilic head group is phosphonic acid, phosphonate, or a salt thereof. In some embodiments of formula (I), the hydrophilic head group is phosphate or a salt thereof. In some embodiments of formula (I), the hydrophilic head group is phosphonate ester or phosphate ester.

Particular examples of hydrophilic head groups of interest include, but are not limited to, a head group comprising a first molecule selected from phosphates (RPO₄H⁻), phosphonates (RPO₃H⁻), boric acid (RBO₂H₂), carboxylates (RCO₂⁻), sulfates (RSO₄⁻), sulfonates (RSO₃⁻), amines (RNH₃⁺), glycerols, sugars such as lactose or derived from hyaluronic acid, polar amino acids, polyethylene oxides and oligoethyleneglycols, that is optionally conjugated to a residue of a second molecule selected from choline, ethanolamine, glycerol, nucleic acid, sugar, inositol, and serine. The head group may contain various other modifications, for instance, in the case of the oligoethyleneglycols and polyethylene oxide (PEG) containing head groups, such PEG chain may be terminated with a methyl group or have a distal functional group for further modification. Examples of hydrophilic head groups also include, but are not limited to, thiophosphate, phosphocholine, phosphoglycerol, phosphoethanolamine, phosphoserine, phosphoinositol, ethylphosphosphorylcholine, polyethyleneglycol, polyglycerol, melamine, glucosamine, trimethylamine, spermine, spermidine, and conjugated carboxylates, sulfates, boric acid, sulfonates, sulfates and carbohydrates.

Any convenient linkers can be utilized to link A to X. In some cases, A is linked to X via a covalent bond. In certain cases, A is linked to X via a linear linker of 1-12 atoms in length, such as 1-10, 1-8 or 1-6 atoms in length, e.g., 1, 2, 3, 4, 5 or 6 atoms in length. The linker L can be a (C₁₋₆)alkyl linker or a substituted (C₁₋₆)alkyl linker, optionally substituted with a heteroatom or linking functional group, such as an ester (—CO₂—), amido (CONH), carbamate (OCONH), ether (—O—), thioether (—S—) and/or amino group (—NR— where R is H or alkyl).

In some instances of formula (I), L is selected from alkyl, substituted alkyl, alkyloxy and substituted alkoxy; and X is selected from phosphonic acid, phosphonate, phosphate, thiophosphate, phosphoramidate and thiophosphoramidate. In some embodiments of formula (I), L-X comprises a group of the formula (XI):

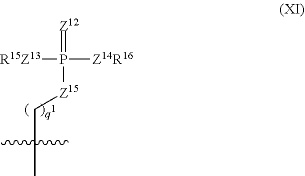

wherein:
Z¹² is selected from O and S;
Z¹³ and Z¹⁴ are each independently selected from O and NR′;
Z¹⁵ is selected from O and CH₂;
R¹⁵ and R¹⁶ are each independently selected from H, alkyl, substituted alkyl, alkenyl, substituted alkenyl, alkoxy, substituted alkoxy, aryl, substituted aryl, an acyl group, an ester, an amide, heterocycle, substituted heterocycle cycloalkyl and substituted cycloalkyl;
R′ is H, alkyl or substituted alkyl; and
q¹ is an integer from 0 to 6.

In some embodiments of formula (XI), Z¹², Z¹³ and Z¹⁴ are all oxygen atoms and Z¹⁵ is CH₂. In other cases, Z¹² is a sulfur atom, Z¹³ and Z¹⁴ are both oxygen atoms and Z¹⁵ is CH₂. In other cases, Z¹² is a sulfur atom, Z¹³, Z¹⁴, Z¹⁵ are all oxygen atoms. In some cases, Z¹² is an oxygen atom, Z¹³ is NR′, Z¹⁴ is an oxygen atom and Z¹⁵ is a carbon atom. In other cases, Z¹² is an oxygen atom, Z¹³ is a nitrogen atom, Z¹⁴ and Z¹⁵ are both oxygen atoms. In other cases, Z¹² is an oxygen atom, Z¹³ and Z¹⁴ are each independently NR′ and Z¹⁵ is an oxygen atom. In yet other cases, Z¹² is an oxygen atom, Z¹³ and Z¹⁴ are each independently NR′ and Z¹⁵ is CH₂.

In some embodiments of formula (XI), R¹⁵ and R¹⁶ are both hydrogen atoms. In other cases, both R¹⁵ and R¹⁶ are substituents other than hydrogen. In some cases, R¹⁵ and R¹⁶ are each independently alkyl or substituted alkyl groups. In some other cases, R¹⁵ and R¹⁶ are each independently aryl groups. In some cases, R¹⁵ and R¹⁶ are each independently alkyl groups. In some cases, R¹⁵ and R¹⁶ are both alkyl groups substituted with an ester. In other cases, R¹⁵ and R¹⁶ are both alkyl groups substituted with an ester. In certain cases, both R¹⁵ and R¹⁶ are phenyl groups. In some cases, R¹⁵ and R¹⁶ are each the same substituent. In other cases, R¹⁵ and R¹⁶ are different substituents.

In some embodiments of formula (XI), Z¹⁵ is a carbon atom and q¹ is 0. In other cases, Z¹⁵ is a carbon atom and q¹ is greater than 0, such as 1, 2, 3, 4, 5 or 6. In some cases, Z¹⁵ is a carbon atom and q¹ is 1. In other embodiments, Z¹⁵ is an oxygen atom and q¹ is 1. In other cases, Z¹⁵ is an oxygen atom and q¹ is greater than 1, such as 2, 3, 4, 5 or 6. In some cases, Z¹⁵ is an oxygen atom and q¹ is 2.

In some embodiments of formula (XI), the L-X is selected from one of the following groups:

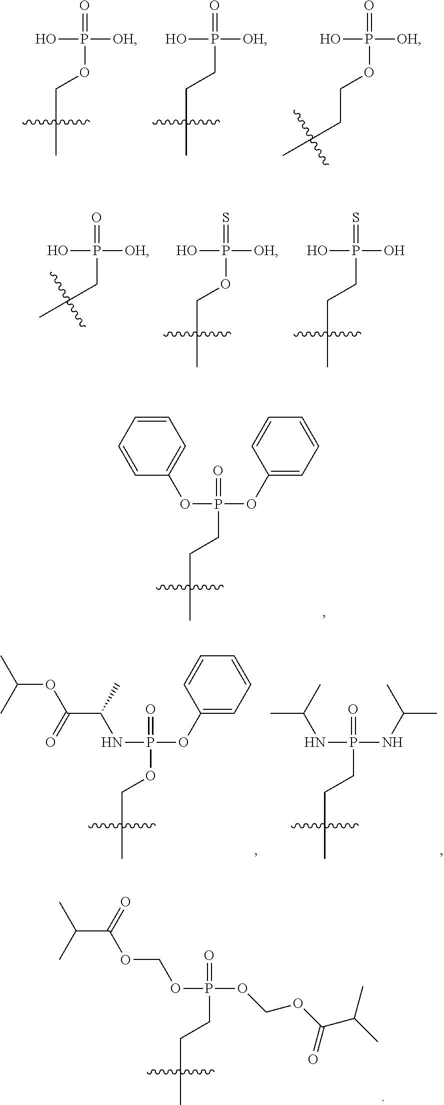

In some embodiments of formula (I), L-X comprises a group of the formula (XII):

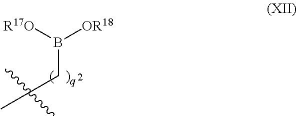

wherein:
R¹⁷ and R¹⁸ are each independently selected from H, alkyl, substituted alkyl, alkenyl, substituted alkenyl, alkoxy, substituted alkoxy, aryl, substituted aryl, an acyl group, an ester, an amide, heterocycle, substituted heterocycle cycloalkyl and substituted cycloalkyl or R¹⁷ and R¹⁸ together with the atoms to which they are attached form a group selected from heterocycle and substituted heterocycle; and
q² is an integer from 1 to 6.

In some embodiments of formula (XII), R¹⁷ and R¹⁸ are both hydrogen atoms. In other cases, both R¹⁷ and R¹⁸ are substituents other than hydrogen. In certain embodiments of formula (XII), q² is 1. In certain cases, q² is greater than 1, such as 2, 3, 4, 5 or 6. In some cases of formula (XII), q² is 2. In certain embodiments of formula (XII), the hydrophilic head group is of the structure:

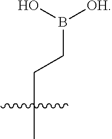 [SMILES: *CCB(O)O]

In some embodiments of formula (I), L-X comprises a group of the formula (XIII):

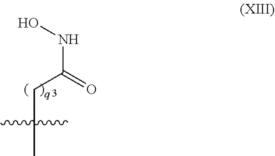

wherein q3 is an integer from 1 to 6. In certain embodiments, q³ is 1. In certain embodiments, q³ is greater than 1, such as 2, 3, 4, 5 or 6. In certain embodiments, q³ is 2. In certain embodiments of formula (XIII), the hydrophilic head group is of the structure:

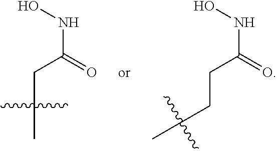 [SMILES: *CC(=O)NO ; *CCC(=O)NO]

In some embodiments of formula (I), L-X comprises a group of the formula (XIV):

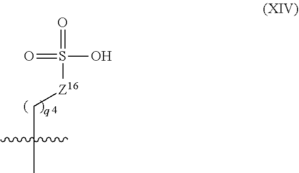

wherein: Z¹⁶ is selected from O and CH₂; and

q¹ is an integer from 0 to 6 (e.g., 0-5).

In some embodiments of formula (XIV), Z¹⁶ is CH₂ and q⁴ is 0. In other cases, Z¹⁶ is CH₂ and q¹ is greater than 0, such as 1, 2, 3, 4, 5 or 6. In some cases, Z¹⁶ is CH₂ and q¹ is 1. In other embodiments, Z¹⁶ is an oxygen atom and q¹ is 1. In other cases, Z¹⁶ is an oxygen atom and q¹ is greater than 1, such as 2, 3, 4, 5 or 6. In some cases, Z¹⁶ is an oxygen atom and q¹ is 2.

In some embodiments of formula (XIV), the hydrophilic head group is selected from one of the following groups:

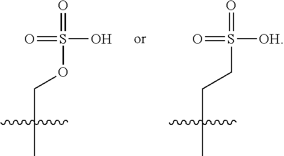 [SMILES: *COS(=O)(=O)O ; *CCS(=O)(=O)O]

In some embodiments of formula (I), L-X comprises a group of the formula (XV):

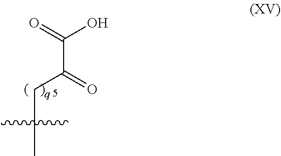

wherein q5 is an integer from 1 to 6. In certain embodiments, q⁵ is 1. In certain embodiments, q⁵ is greater than 1, such as 2, 3, 4, 5 or 6. In certain embodiments, q⁵ is 2. In certain embodiments of formula (XV), the hydrophilic head group is of the structure:

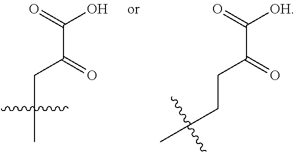 [SMILES: *CC(=O)C(=O)O ; *CCC(=O)C(=O)O]

In some embodiments of formula (I), L-X comprises a group of the formula (XVI):

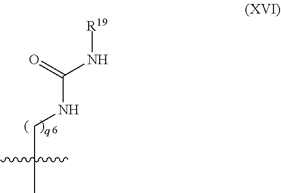

wherein:
R¹⁹ is selected from H, alkyl, substituted alkyl, alkenyl, substituted alkenyl, alkoxy, substituted alkoxy, aryl, substituted aryl, an acyl group, an ester, an amide, heterocycle, substituted heterocycle cycloalkyl and substituted cycloalkyl; and
q⁶ is an integer from 1 to 6.

In some embodiments of formula (XVI), R¹⁹ is hydrogen. In other cases, R¹⁹ is a substituent other than hydrogen. In certain embodiments, R¹⁹ is alkyl or substituted alkyl. In certain embodiments of formula (XVI), q⁶ is 1. In certain cases, q⁶ is greater than 1, such as 2, 3, 4, 5 or 6. In some cases of formula (XVI), q⁶ is 2. In certain embodiments of formula (XVI), the -L-X is of the structure:

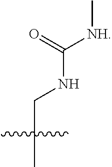 [SMILES: *CNC(=O)NC]

In some embodiments of formula (I), L-X is of the formula (XVII):

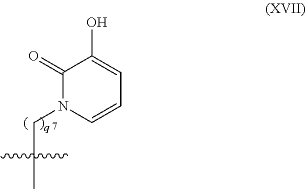

wherein q7 is an integer from 1 to 6. In certain embodiments, q⁷ is 1. In certain embodiments, q⁷ is greater than 1, such as 2, 3, 4, 5 or 6. In certain embodiments, q⁷ is 2. In certain embodiments of formula (XVII), L-X is of the structure:

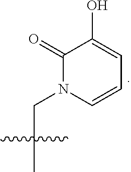 [SMILES: *Cn1cccc(O)c1=O]

In some embodiments of formula (I), A is a heterocycle or substituted heterocycle. In some cases, A is a saturated heterocycle or substituted saturated heterocycle. The heterocycle can be a 5-, 6- or 7-membered monocyclic heterocycle. Heterocycles of interest include, but are not limited to, piperidine, piperazine, morpholine, tetrahydropyran, dioxane, imidazolidine, pyrazolidine, oxazolidine, isoxazolidine, and the like. In certain cases, the heterocycle is a 6-membered ring that is linked to Y and L via a 1, 4-configuration. In certain cases, the heterocycle is a 5- or 6-membered ring that is linked to Y and L via a 1, 3-configuration. In certain cases, the heterocycle is piperidine, substituted piperidine, piperazine or substituted piperazine. When the linking atom of the ring is C, the heterocycle can include a chiral center. In some cases, A is selected from one of the following heterocyclic groups:

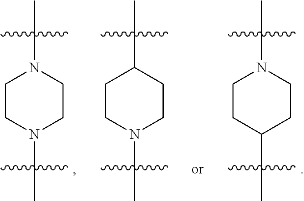

In some embodiments of formula (I), A is a carbocycle. In some cases, A is a saturated carbocycle or substituted saturated carbocycle. The carbocycle can be a 5-, 6- or 7-membered monocyclic carbocycle, such as a cycloalkyl ring. Carbocycle of interest include, but are not limited to, cyclopentane, cyclohexane, cycloheptane, and the like. In certain cases, the carbocycle is a 6-membered ring that is linked to Y and L via a 1, 4-configuration. In certain cases, the carbocycle is a 5- or 6-membered ring that is linked to Y and L via a 1, 3-configuration. In certain cases, the carbocycle is cyclohexane or substituted cyclohexane. The cyclohexane can include a chiral center. In some cases, A is of the structure:

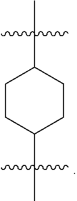 [SMILES: *C1CCC(*)CC1]

In certain other cases, A is an aromatic carbocycle, i.e., aryl. The aryl ring can be monocyclic. In certain cases, A is phenylene or substituted phenylene. In some cases, A is a 1,4-phenylene of the structure:

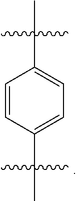 [SMILES: *c1ccc(*)cc1]

In certain other cases, A is an aromatic heterocycle, i.e., heteroaryl or substituted heteroaryl. The heteroaryl ring can be monocyclic. Heteroaryls of interest include, but are not limited to, pyridine, pyridazine, pyrimidine and pyrazine.

In some embodiments of formula (I), L is —(CH₂)n-. In certain cases n is 1 to 8, such as 1 to 5. In some cases, n is 1 to 3, such as 2 or 3. In some cases, n is less than 8, such as 7, 6, 5, 4, 3, 2 or 1. In some cases, n is 1 to 6, such as 1 to 4 or 1 to 3. In some cases, n is 1. In some other cases, n is 2. In some cases, L is an ethylene or substituted ethylene group. In some other cases, L is a methylene or substituted methylene group. In certain other cases L is a covalent bond.

In some embodiments of formula (I), Y is selected from quinazoline, substituted quinazoline, quinoline, substituted quinoline, naphthalene, substituted naphthalene, isoquinoline and substituted isoquinoline. In certain instances, Y is selected from quinazoline and substituted quinazoline. In certain instances, Y is selected from quinoline and substituted quinoline. In certain instances, Y is selected from naphthalene and substituted naphthalene. In certain instances, Y is selected from isoquinoline and substituted isoquinoline. In some embodiments of formula (I), Y is a group of formula (II):

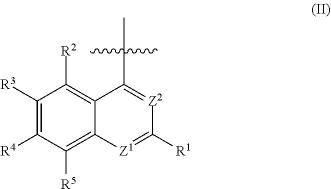

wherein:
Z¹ and Z² are each independently selected from CR¹ and N;
each R¹ is independently selected from H, cyano, trifluoromethyl, halogen, amino, alkyl, substituted alkyl, alkenyl, substituted alkenyl, heterocycle and substituted heterocycle;
R² and R⁵ are each independently selected from H, OH, alkyl, substituted alkyl, alkenyl, substituted alkenyl, alkoxy, substituted alkoxy, —OCF₃, amine, substituted amine, amide, heterocycle and substituted heterocycle; and
R³ and R⁴ are each independently selected from H, OH, alkyl, substituted alkyl, alkenyl, substituted alkenyl, alkoxy, substituted alkoxy, —OCF₃, amine, substituted amine, amide, heterocycle and substituted heterocycle; or R³ and R⁴ together with the carbon atoms to which they are attached form a fused ring selected from heterocycle, substituted heterocycle, cycloalkyl, substituted cycloalkyl, aryl and substituted aryl.

In certain embodiments of formula (II), at least of Z¹ and Z² is N. In certain embodiments of formula (II), Z¹ is C and Z² is N. In certain cases of formula (II), Z¹ is N and Z² is C. In certain instances of formula (IIa), Z¹ is C and Z² is C. In certain cases of formula (II), Z¹ is N and Z² is N. In some instances of formula (II), R¹ and R⁴ are not hydrogen. In some instances of formula (II), R¹, R³ and R⁴ are not hydrogen. In some instances of formula (II), R¹, R³, R⁴ and R⁵ are not hydrogen.

In some instances of formula (II), R¹ is selected from hydrogen, C₁₋₅ alkyl, vinyl heterocycle (e.g., —CH═CH-heterocycle). In certain instances, the -vinyl heterocycle is vinyl pyridine (e.g., —CH═CH-pyridine). In some instances of formula (IIa), R¹ is hydrogen. In some cases, R¹ is C₁₋₅ alkyl. In other cases R¹ is a vinyl heterocycle. In certain cases, R¹ is vinyl pyridine. In some instances, R² and R⁵ are both hydrogen. In some cases, R⁵ is selected from C₁₋₅ alkyl, amine, triazole, imidazole, amide, alkoxy, OCF₃ and hydroxy. In certain cases, R⁵ is alkoxy, e.g., methoxy. In some instances, R³ and R⁴ are each independently selected from hydrogen, C₁₋₅ alkyl, triazole, imidazole, amine, amide, alkoxy, OCF₃, hydroxy, or R³ and R⁴ together with the carbon to which they are attached from a heterocycle. In some cases, R³ and R⁴ are alkoxy, e.g., in some cases R³ and R⁴ are both methoxy. In some cases, R⁵ is methoxy and each of R¹-R⁴ are hydrogen. In some cases, R⁵ is methoxy, R¹ is —CH═CH-heterocycle and each of R²-R⁴ are hydrogen.

In some embodiments of formula (II), Y is a group of formula (IIA):

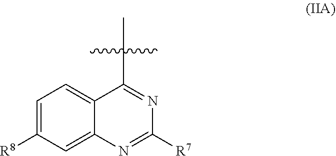

wherein,
R⁷ is selected from the group consisting of H, alkyl, substituted alkyl, alkenyl, substituted alkenyl, heterocycle and substituted heterocycle;
R⁸ is selected from the group consisting of OH, alkyl, substituted alkyl, alkenyl, substituted alkenyl, alkoxy, substituted alkoxy, —OCF₃, amine, substituted amine, amide, heterocycle and substituted heterocycle.

In some instances of formula (IIA), R⁷ is selected from hydrogen, C₁₋₅ alkyl, substituted C₁₋₅ alkyl, vinyl-heterocycle and substituted vinyl-heterocycle. In some instances of formula (IIA), R⁷ is selected from hydrogen, C₁₋₅ alkyl, vinyl heterocycle (e.g., —CH═CH-heterocycle). In certain instances, the -vinyl heterocycle is vinyl pyridine (e.g., —CH═CH-pyridine). In some instances of formula (IIA), R⁷ is hydrogen. In some cases, R⁷ is C₁₋₅ alkyl. In other cases, R⁷ is a vinyl heterocycle. In certain cases, R⁷ is vinyl pyridine. In some instances, R⁸ is selected from hydrogen, C₁₋₅ alkyl, triazole, imidazole, amine, amide, alkoxy, OCF₃ and hydroxyl. In some cases, R⁸ is alkoxy, e.g., methoxy. In some cases, R⁸ is methoxy and R⁷ is hydrogen. In some cases, R⁸ is methoxy and R⁷ is —CH═CH-heterocycle. In some embodiments of formula (II), Y is a group of formula (IIB):

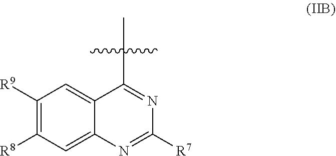

wherein,
R⁷ is selected from the group consisting of H, alkyl, substituted alkyl, alkenyl, substituted alkenyl, heterocycle and substituted heterocycle;
R⁸ and R⁹ are each independently selected from the group consisting of OH, alkyl, substituted alkyl, alkenyl, substituted alkenyl, alkoxy, substituted alkoxy, —OCF₃, amine, substituted amine, amide, heterocycle and substituted heterocycle; or R⁸ and R⁹ together with the carbon atoms to which they are attached form a fused ring selected from heterocycle, substituted heterocycle, cycloalkyl, substituted cycloalkyl, aryl and substituted aryl.

In some instances of formula (IIB), R⁷ is selected from hydrogen, C₁₋₅ alkyl, vinyl heterocycle (e.g., —CH═CH-heterocycle). In certain instances, the -vinyl heterocycle is vinyl pyridine (e.g., —CH═CH-pyridine). In some instances of formula (IIB), R⁷ is hydrogen. In some cases, R⁷ is C₁₋₅ alkyl. In other cases R⁷ is a vinyl heterocycle. In certain cases, R⁷ is vinyl pyridine. In some instances, R⁸ and R⁹ are each independently selected from hydrogen, C₁₋₅ alkyl, triazole, imidazole, amine, amide, alkoxy, OCF₃ and hydroxy, or R⁸ and R⁹ together with the carbon atoms to which they are attached from a fused heterocycle. In some cases, R⁸ and R⁹ are alkoxy, e.g., in some cases R⁸ and R⁹ are both methoxy. In some embodiments of formula (II), Y is a group of formula (IIC):

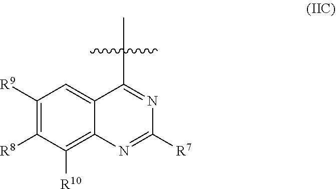

wherein,
R⁷ is selected from the group consisting of H, alkyl, substituted alkyl, alkenyl, substituted alkenyl, heterocycle and substituted heterocycle;
R¹⁰ is selected from the group consisting of OH, alkyl, substituted alkyl, alkenyl, substituted alkenyl, alkoxy, substituted alkoxy, —OCF₃, amine, substituted amine, amide, heterocycle and substituted heterocycle;
R⁸ and R⁹ are each independently selected from the group consisting of OH, alkyl, substituted alkyl, alkenyl, substituted alkenyl, alkoxy, substituted alkoxy, —OCF₃, amine, substituted amine, amide, heterocycle and substituted heterocycle; or R⁸ and R⁹ together with the carbon atoms to which they are attached form a fused ring selected from heterocycle, substituted heterocycle, cycloalkyl, substituted cycloalkyl, aryl and substituted aryl.

In some instances of formula (IIC), R⁷ is selected from hydrogen, C₁₋₅ alkyl, vinyl heterocycle (e.g., —CH═CH-heterocycle). In certain instances, the -vinyl heterocycle is vinyl pyridine (e.g., —CH═CH-pyridine). In some instances of formula (IIC), R⁷ is hydrogen. In some cases, R⁷ is C₁₋₅ alkyl. In some cases, R⁷ is a vinyl heterocycle. In certain cases, R⁷ is vinyl pyridine. In some cases, R¹⁰ is selected from hydrogen, C₁₋₅ alkyl, amine, triazole, imidazole, amide, alkoxy, OCF₃ and hydroxy. In some cases, R¹⁰ is hydrogen. In certain cases, R¹⁰ is alkoxy, e.g., methoxy. In some instances, R⁸ and R⁹ are each independently selected from hydrogen, C₁₋₅ alkyl, triazole, imidazole, amine, amide, alkoxy, OCF₃, hydroxy, or R⁸ and R⁹ together with the carbon atoms to which they are attached from a fused heterocycle. In some cases, R⁸ and R⁹ are alkoxy, e.g., in some cases R⁸ and R⁹ are both methoxy. In some cases, R¹⁰ is methoxy and each of R⁷-R⁹ are hydrogen. In some cases, R¹⁰ is methoxy, R⁷ is —CH═CH-heterocycle and each of R⁸ and R⁹ are hydrogen. In some embodiments of formula (II), Y is a group of formula (IID):

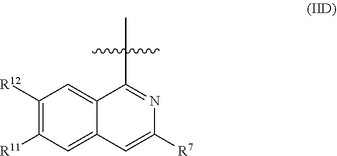

wherein,
R⁷ is selected from the group consisting of H, alkyl, substituted alkyl, alkenyl, substituted alkenyl, heterocycle and substituted heterocycle;
R¹¹ and R¹² are each independently selected from the group consisting of H, OH, alkyl, substituted alkyl, alkenyl, substituted alkenyl, alkoxy, substituted alkoxy, —OCF₃, amine, substituted amine, amide, heterocycle and substituted heterocycle; or R¹¹ and R¹² together with the carbon atoms to which they are attached form a fused ring selected from heterocycle, substituted heterocycle, cycloalkyl, substituted cycloalkyl, aryl and substituted aryl.

In some instances of formula (IID), R⁷ is selected from hydrogen, C₁₋₅ alkyl, vinyl heterocycle (e.g., —CH═CH-heterocycle). In certain instances, the -vinyl heterocycle is vinyl pyridine (e.g., —CH═CH-pyridine). In some instances of formula (IID), R⁷ is hydrogen. In some cases, R⁷ is C₁₋₅ alkyl. In some cases, R⁷ is a vinyl heterocycle. In certain cases, R⁷ is vinyl pyridine. In some instances, R¹¹ and R¹² are each independently selected from hydrogen, C₁₋₅ alkyl, triazole, imidazole, amine, amide, alkoxy, OCF₃ and hydroxy, or R¹¹ and R¹² together with the carbon atoms to which they are attached from a fused heterocycle. In some cases, R¹¹ and R¹² are alkoxy, e.g., in some cases R¹¹ and R¹² are both methoxy.

In some embodiments of formula (II), Y is a group of formula (IIE):

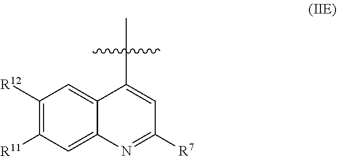

wherein,
R⁷ is selected from the group consisting of H, alkyl, substituted alkyl, alkenyl, substituted alkenyl, heterocycle and substituted heterocycle;
R¹¹ and R¹² are each independently selected from the group consisting of H, OH, alkyl, substituted alkyl, alkenyl, substituted alkenyl, alkoxy, substituted alkoxy, —OCF₃, amine, substituted amine, amide, heterocycle and substituted heterocycle; or R¹¹ and R¹² together with the carbon atoms to which they are attached form a fused ring selected from heterocycle, substituted heterocycle, cycloalkyl, substituted cycloalkyl, aryl and substituted aryl.

In some instances of formula (IIE), R⁷ is selected from hydrogen, C₁₋₅ alkyl, vinyl heterocycle (e.g., —CH═CH-heterocycle). In certain instances, the -vinyl heterocycle is vinyl pyridine (e.g., —CH═CH-pyridine). In some instances of formula (IIE), R⁷ is hydrogen. In some cases, R⁷ is C₁₋₅ alkyl. In other cases R⁷ is a vinyl heterocycle. In certain cases, R⁷ is vinyl pyridine. In some instances, R¹¹ and R¹² are each independently selected from hydrogen, C₁₋₅ alkyl, triazole, imidazole, amine, amide, alkoxy, OCF₃ and hydroxy, or R¹¹ and R¹² together with the carbon to which they are attached from a heterocycle. In some cases, R¹¹ and R¹² are alkoxy, e.g., in some cases R¹¹ and R¹² are both methoxy.

In some embodiments of formula (II), Y is a group selected from:

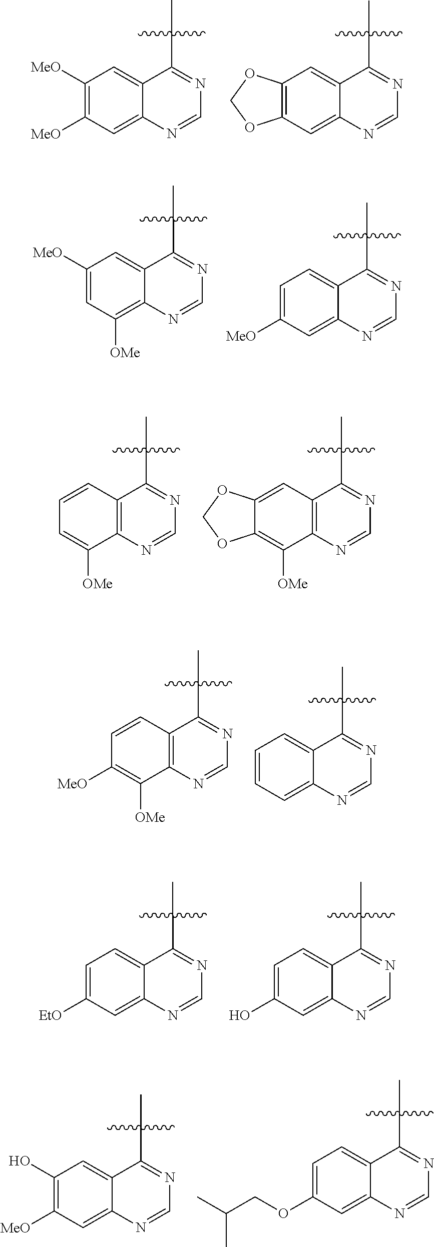

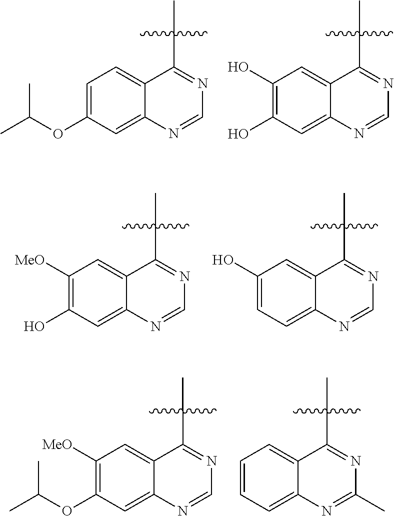

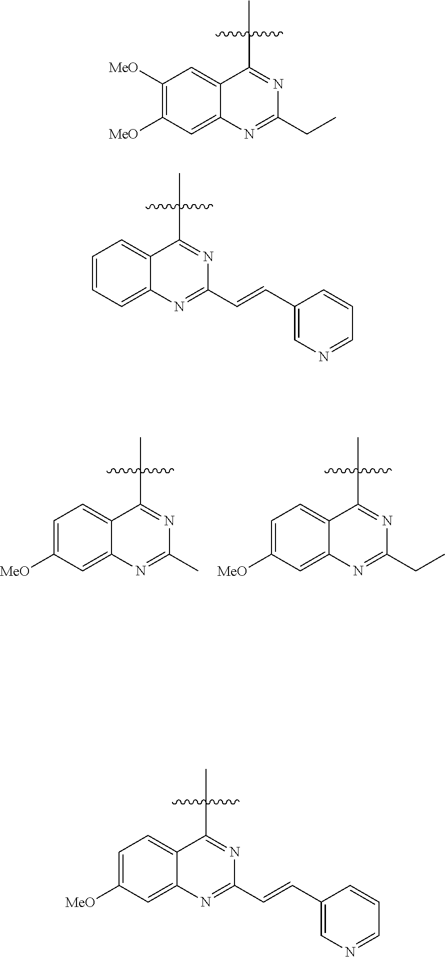

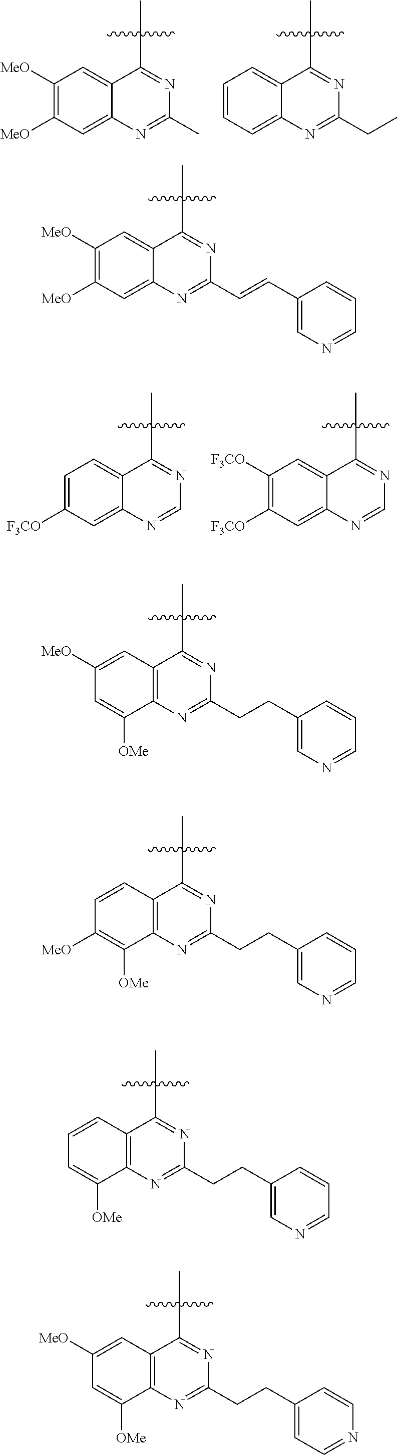

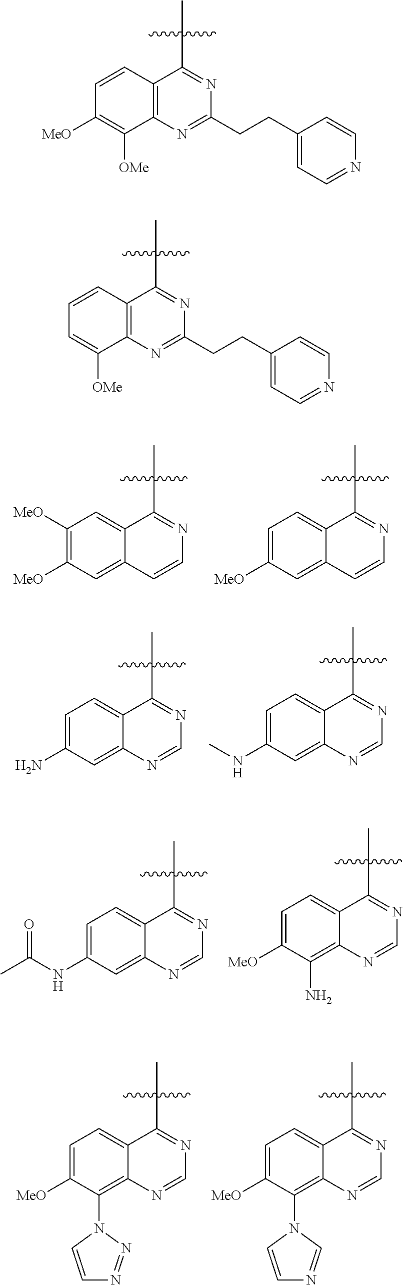

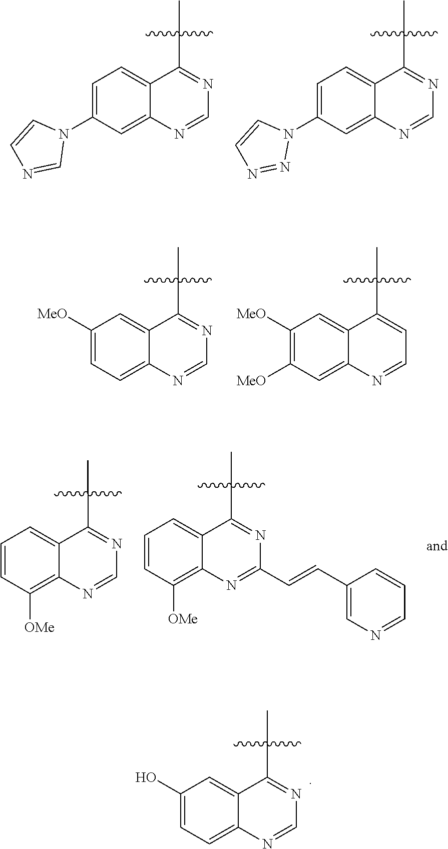

In some embodiments of formula (II), any of R¹ to R⁵ may be a halogen, e.g., F, Cl, Br or I. In some embodiments of formula (II), at least one of R¹ to R⁵ is a halogen atom. In some embodiments of formula (II), at least one of R¹ to R⁵ is fluoride. In other embodiments of formula (II), at least one of R¹ to R⁵ is chloride. In other embodiments of formula (II), at least one of R¹ to R⁵ is bromide. In yet other embodiments of formula (II), at least one of R¹ to R⁵ is iodide.

In some embodiments of formula (II), Y is a group selected from:

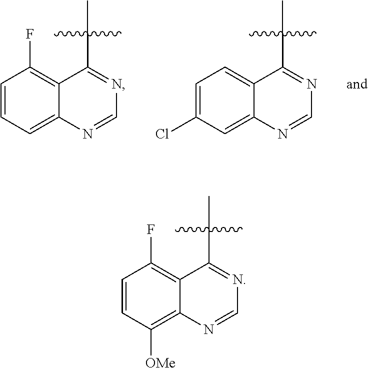 [SMILES: *c1ncnc2cccc(F)c12 ; *c1ncnc2cc(Cl)ccc12 ; "and" ; *c1ncnc2c(OC)ccc(F)c12]

In some embodiments of formula (I), Y is a group of formula (XI):

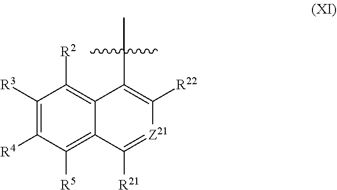

wherein:
Z²¹ is selected from CR¹ and N;
R¹, R²¹ and R²² are independently selected from H, alkyl, substituted alkyl, alkenyl, substituted alkenyl, heterocycle and substituted heterocycle;
R² and R⁵ are independently selected from H, OH, alkyl, substituted alkyl, alkenyl, substituted alkenyl, alkoxy, substituted alkoxy, —OCF₃, amine, substituted amine, amide, heterocycle and substituted heterocycle; and
R³ and R⁴ are independently selected from H, OH, alkyl, substituted alkyl, alkenyl, substituted alkenyl, alkoxy, substituted alkoxy, —OCF₃, amine, substituted amine, amide, heterocycle and substituted heterocycle; or R³ and R⁴ together with the carbon to which they are attached form a fused ring selected from heterocycle, substituted heterocycle, cycloalkyl, substituted cycloalkyl, aryl and substituted aryl.

In some instances of formula (XI), R¹ and R⁴ are not hydrogen. In some instances of formula (XI), R¹, R³ and R⁴ are not hydrogen. In some instances of formula (XI), R¹, R³, R⁴ and R⁵ are not hydrogen.

In some instances of formula (XI), Z²¹ is CR¹ and R¹ is selected from hydrogen, C₁₋₅ alkyl, vinyl heterocycle (e.g., —CH═CH-heterocycle). In certain instances, the -vinyl heterocycle is vinyl pyridine (e.g., —CH═CH-pyridine). In some instances of formula (XI), Z²¹ is CR¹ and R¹ is hydrogen. In some cases, R¹ is C₁₋₅ alkyl. In other cases, Z²¹ is CR¹ and R¹ is a vinyl heterocycle. In certain cases, R¹ is vinyl pyridine. In some instances, R² and R⁵ are both hydrogen. In some cases, R⁵ is selected from C₁₋₅ alkyl, amine, triazole, imidazole, amide, alkoxy, OCF₃ and hydroxy. In certain cases, R⁵ is alkoxy, e.g., methoxy. In some instances, R³ and R⁴ are each independently selected from hydrogen, C₁₋₅ alkyl, triazole, imidazole, amine, amide, alkoxy, OCF₃, hydroxy, or R³ and R⁴ together with the carbon to which they are attached from a heterocycle. In some cases, R³ and R⁴ are alkoxy, e.g., in some cases R³ and R⁴ are both methoxy. In some cases, R⁵ is methoxy and each of R¹-R⁴ are hydrogen. In some cases, R⁵ is methoxy, R¹ is —CH═CH-heterocycle and each of R²-R⁴ are hydrogen.

In some embodiments of formula (I), Y is a group of the formula (III):

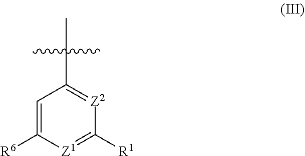

wherein:
Z¹ and Z² are each independently selected from CR¹ and N;
each R¹ is independently selected from the group consisting of H, alkyl, substituted alkyl, alkenyl, substituted alkenyl, heterocycle and substituted heterocycle; and
R⁶ is selected from the group consisting of heterocycle, substituted heterocycle, cycloalkyl, substituted cycloalkyl, aryl and substituted aryl. In certain embodiments of formula (III), at least of Z¹ and Z² is N. In certain embodiments of formula (III), Z¹ is CH and Z² is N. In certain cases of formula (III), Z¹ is N and Z² is CH. In certain instances of formula (III), Z¹ is CH and Z² is CH. In certain cases of formula (III), Z¹ is N and Z² is N.

In some embodiments of formula (III), Y is a group of the formula (IIIA):

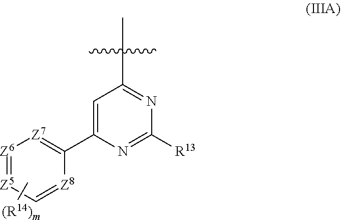

wherein,
Z⁵, Z⁶, Z⁷ and Z⁸ are each independently selected from CR¹⁴ and N;
R¹³ is selected from the group consisting of H, alkyl, substituted alkyl, alkenyl, substituted alkenyl, heterocycle and substituted heterocycle;
each R¹⁴ is independently selected from the group consisting of H, OH, alkyl, substituted alkyl, alkenyl, substituted alkenyl, alkoxy, substituted alkoxy, —OCF₃, amine, substituted amine, amide, heterocycle and substituted heterocycle; and
m is 0-5.
   In some instance of formula (IIIA), one and only one of Z⁵, Z⁶, Z⁷ and Z⁸ is N. In some instance of formula (IIIA), two and only two of Z⁵, Z⁶, Z⁷ and Z⁸ are N. In some instance of formula (IIIA), Z⁵ is N. In some instance of formula (IIIA), Z⁶ is N. In some instance of formula (IIIA), Z⁷ is N. In some instance of formula (IIIA), Z⁸ is N. In some instance of formula (IIIA), Z⁵ and Z⁷ are each N. In some instance of formula (IIIA), Z⁷ and Z⁸ are each N.

In some embodiments of formula (III), Y is a group of the formula (IIIB):

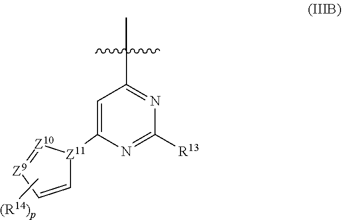

wherein,
Z⁹, Z¹⁰ and Z¹¹ are each independently selected from CR¹⁴ and N;
R¹³ is selected from the group consisting of H, alkyl, substituted alkyl, alkenyl, substituted alkenyl, heterocycle and substituted heterocycle;
each R¹⁴ is independently selected from the group consisting of H, OH, alkyl, substituted alkyl, alkenyl, substituted alkenyl, alkoxy, substituted alkoxy, —OCF₃, amine, substituted amine, amide, heterocycle and substituted heterocycle; and
p is 0-4.
   In some instance of formula (IIIB), one and only one of Z⁹, Z¹⁰ and Z¹¹ is N. In some instance of formula (IIIB), two and only two of Z⁹, Z¹⁰ and Z¹¹ are N. In some instance of formula (IIIB), Z⁹ is N. In some instance of formula (IIIA), Z¹⁰ is N. In some instance of formula (IIIB), Z¹¹ is N. In some instances of formula (IIIB), R¹⁴ is selected form alkyl and substituted alkyl. In some instances of formula (IIIB), p is 0. In some instances of formula (IIIB), p is 1. In some instances of formula (IIIB), p is 2.

In some embodiments of formula (III), Y is a group selected from:

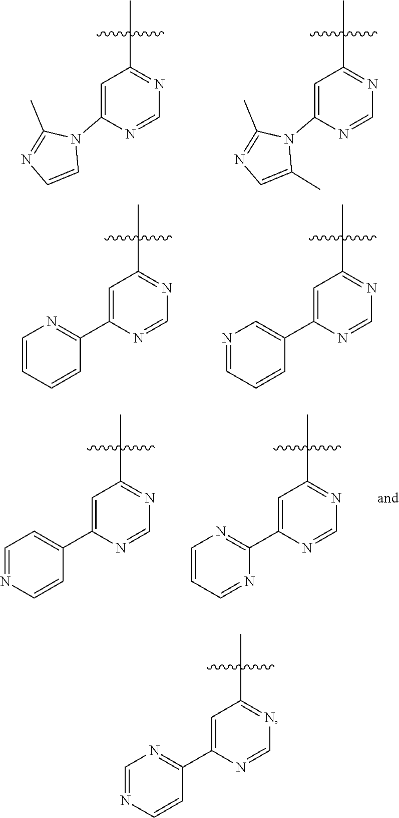

or a substituted version thereof.

In some embodiments of formula (I), Y is a group of formula (IIIC)

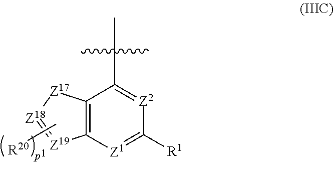

wherein,
Z¹, Z², Z¹⁷, Z¹⁸ and Z¹⁹ are each independently selected from CR²⁰ and N;
each R²⁰ is independently selected from the group consisting of H, OH, alkyl, substituted alkyl, alkenyl, substituted alkenyl, alkoxy, substituted alkoxy, —OCF₃, amine, substituted amine, amide, heterocycle and substituted heterocycle; and
p¹ is an integer from 0-4.
   In some instances of formula (IIIC), Z¹, Z², Z¹⁷ and Z¹⁹ are each N and Z¹⁸ is CR²⁰.

In some embodiments of formula (IIIC), Y is of the structure:

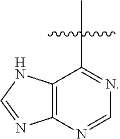 [SMILES: *c1ncnc2nc[nH]c12]

In some embodiments of formula (I), the structure has the formula (IV):

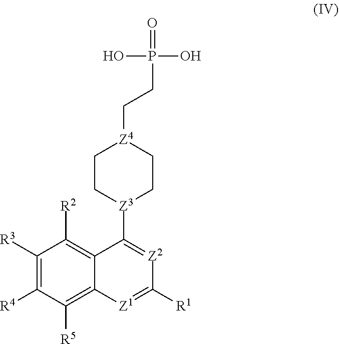

wherein,
Z¹ and Z² are each independently selected from CR¹ and N;
Z³ and Z⁴ are each independently selected from CR and N, where R is H, alkyl or substituted alkyl;
R¹ is selected from the group consisting of H, alkyl, substituted alkyl, alkenyl, substituted alkenyl, heterocycle and substituted heterocycle;
R² and R⁵ are each independently selected from the group consisting of H, OH, alkyl, substituted alkyl, alkenyl, substituted alkenyl, alkoxy, substituted alkoxy, —OCF₃, amine, substituted amine, amide, heterocycle and substituted heterocycle;
R³ and R⁴ are each independently selected from the group consisting of H, OH, alkyl, substituted alkyl, alkenyl, substituted alkenyl, alkoxy, substituted alkoxy, —OCF₃, amine, substituted amine, amide, heterocycle and substituted heterocycle;
or R³ and R⁴ together with the carbon to which they are attached form a group selected from heterocycle, substituted heterocycle, cycloalkyl, substituted cycloalkyl, aryl and substituted aryl,
or a pro-drug, a pharmaceutically acceptable salt or a solvate thereof.

In certain embodiments of formula (IV), at least one of Z¹ and Z² is N. In certain embodiments of formula (IV), Z¹ is C and Z² is N. In certain cases of formula (IV), Z¹ is N and Z² is C. In certain instances of formula (IV), Z¹ is C and Z² is C. In certain cases of formula (IV), Z¹ is N and Z² is N. In certain embodiments of formula (IV), at least one of Z³ and Z⁴ is N. In certain cases of formula (IV), Z³ is N and Z⁴ is N. In certain cases of formula (IV), Z³ is N and Z⁴ is CH. In certain cases of formula (IV), Z³ is CH and Z⁴ is N. In certain cases of formula (VI), Z³ is CH and Z⁴ is CH.

In some instances of formula (IV), R¹ is selected from hydrogen, C₁₋₅ alkyl, vinyl heterocycle (e.g., —CH═CH-heterocycle). In certain instances, the -vinyl heterocycle is vinyl pyridine (e.g., —CH═CH-pyridine). In some instances of formula (IV), R¹ is hydrogen. In some cases, R¹ is C₁₋₅ alkyl. In other cases, R¹ is a vinyl heterocycle. In certain cases, R¹ is vinyl pyridine. In some instances, R² and R⁵ are both hydrogen. In some cases, R⁵ is selected from C₁₋₅ alkyl, amine, triazole, imidazole, amide, alkoxy, OCF₃ and hydroxy. In certain cases, R⁵ is alkoxy, e.g., methoxy. In some instances, R³ and R⁴ are each independently selected from hydrogen, C₁₋₅ alkyl, triazole, imidazole, amine, amide, alkoxy, OCF₃, hydroxy, or R³ and R⁴ together with the carbon to which they are attached from a heterocycle. In some cases, R³ and R⁴ are alkoxy, e.g., in some cases R³ and R⁴ are both methoxy. In some cases, R⁵ is methoxy and each of R¹-R⁴ are hydrogen. In some cases, R⁵ is methoxy, R¹ is —CH═CH-heterocycle and each of R²-R⁴ are hydrogen.

In some embodiments of formula (I), the structure has the formula (V)

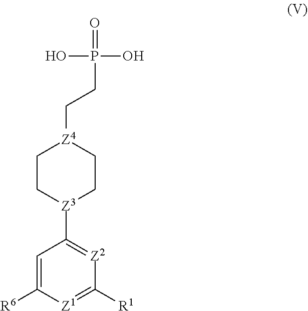

wherein:
Z¹ and Z² are each independently selected from CR¹ and N;
Z³ and Z⁴ are each independently selected from CR and N, where R is H, alkyl or substituted alkyl;
each R¹ is independently selected from H, alkyl, substituted alkyl, alkenyl, substituted alkenyl, heterocycle and substituted heterocycle;
R⁶ is selected from heterocycle, substituted heterocycle, cycloalkyl, substituted cycloalkyl, aryl and substituted aryl,
or a pro-drug, a pharmaceutically acceptable salt or a solvate thereof.

In certain embodiments of formula (V), at least one of Z¹ and Z² is N. In certain embodiments of formula (V), Z¹ is CH and Z² is N. In certain cases of formula (IV), Z¹ is N and Z² is CH. In certain instances of formula (V), Z¹ is CH and Z² is CH. In certain cases of formula (IV), Z¹ is N and Z² is N. In certain embodiments of formula (V), at least one of Z³ and Z⁴ is N. In certain cases of formula (V), Z³ is N and Z⁴ is N. In certain cases of formula (V), Z³ is N and Z⁴ is CH. In certain cases of formula (V), Z³ is CH and Z⁴ is N. In certain cases of formula (V), Z³ is CH and Z⁴ is CH.

In some embodiments of formula (I), the inhibitor has formula (VI):

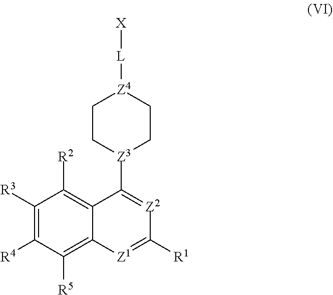

wherein,
X is a hydrophilic head group selected from phosphonic acid, phosphonate, phosphonate ester, phosphate, phosphate ester, thiophosphate, thiophosphate ester, phosphoramidate and thiophosphoramidate;
L is a linker;
Z¹ and Z² are each independently selected from CR¹ and N;
Z³ and Z⁴ are each independently selected from CR and N, wherein R is H, alkyl or substituted alkyl;
each R¹ is independently selected from H, alkyl, substituted alkyl, alkenyl, substituted alkenyl, heterocycle and substituted heterocycle;
R² and R⁵ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle;
R³ and R⁴ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle; or R³ and R⁴ together with the carbon atoms to which they are attached form a fused ring selected from heterocycle, substituted heterocycle, cycloalkyl, substituted cycloalkyl, aryl and substituted aryl;
or a pro-drug, a pharmaceutically acceptable salt or a solvate thereof.

In some embodiments of formula (I), the structure has the formula (VI):

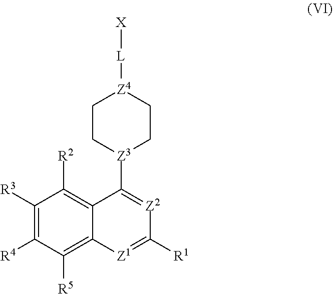

wherein,
X is a hydrophilic head group selected from boronic acid, hydroxamic acid, phosphonic acid, phosphonate, phosphonate ester, phosphate, phosphate ester, thiophosphate, thiophosphate ester, phosphoramidate and thiophosphoramidate;
L is a linker;
Z¹ and Z² are each independently selected from CR¹ and N;
Z³ and Z⁴ are each independently selected from CR and N, wherein R is H, alkyl or substituted alkyl;
each R¹ is independently selected from H, cyano, trifluoromethyl, halogen, amino, alkyl, substituted alkyl, alkenyl, substituted alkenyl, heterocycle and substituted heterocycle;
R² and R⁵ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle;
R³ and R⁴ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle; or R³ and R⁴ together with the carbon atoms to which they are attached form a fused selected from heterocycle, substituted heterocycle, cycloalkyl, substituted cycloalkyl, aryl and substituted aryl;
or a pro-drug, a pharmaceutically acceptable salt or a solvate thereof.

In some embodiments of formula (I), the structure has the formula (VI):

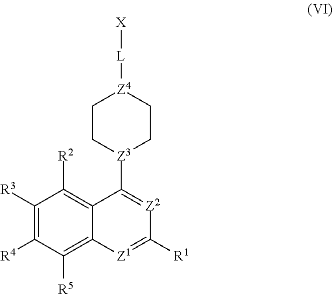

wherein,
X is a hydrophilic head group selected from phosphonic acid, phosphonate, phosphonate ester, phosphate, phosphate ester, thiophosphate, thiophosphate ester, phosphoramidate and thiophosphoramidate;
L is selected from —CH₂—, —(CH₂)₂—, —(CH₂)₃—, —(CH₂)₄—, —(CH₂)₅— and —(CH₂)₆—;
Z¹ and Z² are each independently selected from CR¹ and N;
Z³ and Z⁴ are each independently selected from CR and N, wherein R is H, alkyl or substituted alkyl;
each R¹ is independently selected from H, alkyl, substituted alkyl, alkenyl, substituted alkenyl, heterocycle and substituted heterocycle;
R² and R⁵ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle;
R³ and R⁴ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle; or R³ and R⁴ together with the carbon atoms to which they are attached form a fused ring selected from heterocycle, substituted heterocycle, cycloalkyl, substituted cycloalkyl, aryl and substituted aryl;
or a pharmaceutically acceptable salt or a solvate thereof.

In some embodiments of formula (I), the structure has the formula (VI):

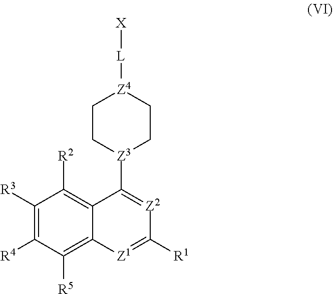

wherein,
L is selected from the group consisting of —CH₂—, —(CH₂)₂—, —(CH₂)₃—, —(CH₂)₄—, —(CH₂)₅— and —(CH₂)₆—;
X is selected from the group consisting of

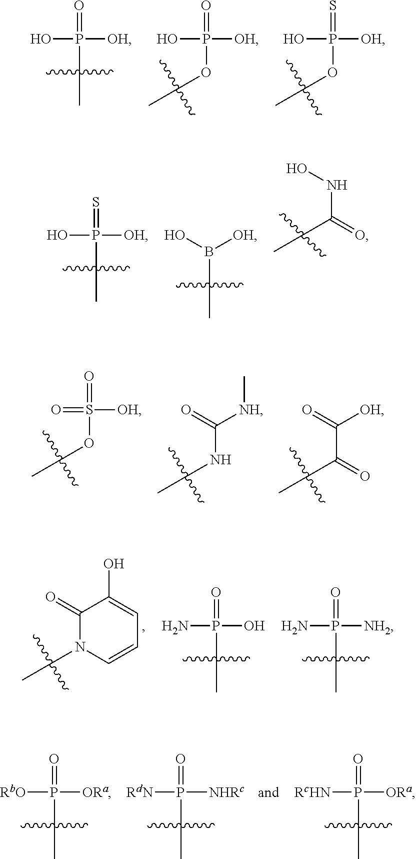

wherein R^{a} and R^{b} are each independently selected from aryl, alkyl, —CH₂OC(O)R^{e}, —CH₂OC(O)OR^{e}; R^{c} and R^{d} are each independently selected from —C(CH₃)C(O)ORe, alkyl and wherein R^{e} is alkyl;
Z¹, Z², Z³ and Z⁴ are each independently selected from CR¹ and N;
R¹ is selected from the group consisting of H, alkyl, substituted alkyl, alkenyl, substituted alkenyl, heterocycle and substituted heterocycle;
R² and R⁵ are each independently selected from the group consisting of H, OH, alkyl, substituted alkyl, alkenyl, substituted alkenyl, alkoxy, substituted alkoxy, —OCF₃, amine, substituted amine, amide, heterocycle and substituted heterocycle;
R³ and R⁴ are each independently selected from the group consisting of H, OH, alkyl, substituted alkyl, alkenyl, substituted alkenyl, alkoxy, substituted alkoxy, —OCF₃, amine, substituted amine, amide, heterocycle and substituted heterocycle;
or R³ and R⁴ together with the carbon to which they are attached form a group selected from heterocycle, substituted heterocycle, cycloalkyl, substituted cycloalkyl, aryl and substituted aryl,
or a pro-drug, a pharmaceutically acceptable salt or a solvate thereof.

In certain embodiments of formula (VI), at least one of Z¹ and Z² is N. In certain embodiments of formula (VI), Z¹ is C and Z² is N. In certain cases of formula (VI), Z¹ is N and Z² is C. In certain instances of formula (VI), Z¹ is C and Z² is C. In certain cases of formula (VI), Z¹ is N and Z² is N. In certain embodiments of formula (VI), at least one of Z³ and Z⁴ is N. In certain cases of formula (VI), Z³ is N and Z⁴ is N. In certain cases of formula (IVI Z³ is N and Z⁴ is C. In certain cases of formula (VI), Z³ is C and Z⁴ is N. In certain cases of formula (VI), Z³ is C and Z⁴ is C.

In some instances of formula (VI), R¹ is selected from hydrogen, C₁₋₅ alkyl, vinyl heterocycle (e.g., —CH═CH-heterocycle). In certain instances, the -vinyl heterocycle is vinyl pyridine (e.g., —CH═CH-pyridine). In some instances of formula (VI), R¹ is hydrogen. In some cases, R¹ is C₁₋₅ alkyl. In other cases R¹ is a vinyl heterocycle. In certain cases, R¹ is vinyl pyridine. In some instances, R² and R⁵ are both hydrogen. In some cases, R⁵ is selected from C₁₋₅ alkyl, amine, triazole, imidazole, amide, alkoxy, OCF₃ and hydroxy. In certain cases, R⁵ is alkoxy, e.g., methoxy. In some instances, R³ and R⁴ are each independently selected from hydrogen, C₁₋₅ alkyl, triazole, imidazole, amine, amide, alkoxy, OCF₃, hydroxy, or R³ and R⁴ together with the carbon to which they are attached from a heterocycle. In some cases, R³ and R⁴ are alkoxy, e.g., in some cases R³ and R⁴ are both methoxy. In some cases, R⁵ is methoxy and each of R¹-R⁴ are hydrogen. In some cases, R⁵ is methoxy, R¹ is —CH═CH-heterocycle and each of R²-R⁴ are hydrogen.

In certain embodiments of formula (VI), L is —CH₂—. In certain other cases of formula (VI), L is —(CH₂)₂—.

In certain embodiments of formula (VI), X is

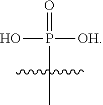 [SMILES: *P(=O)(O)O]

In certain cases of formula (VI), X is

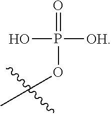 [SMILES: *OP(=O)(O)O]

In certain other cases of formula (VI), X is

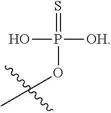 [SMILES: *OP(O)(O)=S]

In certain cases of formula (VI), X is

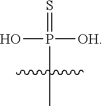 [SMILES: *P(O)(O)=S]

In certain other cases of formula (VI), X is

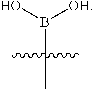 [SMILES: *B(O)O]

In certain embodiments of formula (VI), X is

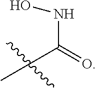 [SMILES: *C(=O)NO]

In certain cases of formula (VI), X is

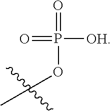

In certain other cases of formula (VI), X is

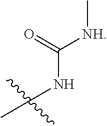 [SMILES: *NC(=O)NC]

In certain cases of formula (VI), X is

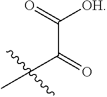 [SMILES: *C(=O)C(=O)O]

In certain other cases of formula (VI), X is

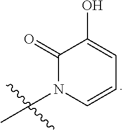 [SMILES: *n1cccc(O)c1=O]

In certain cases of formula (VI), X is

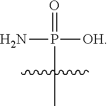 [SMILES: *P(N)(=O)O]

In certain other cases of formula (VI), X is

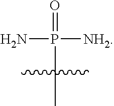 [SMILES: *P(N)(N)=O]

In certain other cases of formula (VI), X is

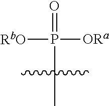

wherein R^{a} and R^{b} are each independently selected from aryl, alkyl, —CH₂OC(O)R^{e}, —CH₂OC(O)OR^{e}, wherein R^{e} is alkyl. In certain cases of formula (VI), X is

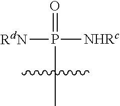

wherein R^{c} and R^{d} are each independently selected from —C(CH₃)C(O)Ore and alkyl, wherein R^{e} is alkyl. In certain other cases of formula (VI), X is

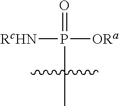

wherein R^{a} is selected from aryl, alkyl, —CH₂OC(O)R^{e}, —CH₂OC(O)OR^{e} and R^{c} is selected from —C(CH₃)C(O)Ore and alkyl, wherein R^{e} is alkyl.

It will be understood that any of the hydroxyl and amine groups in group X in formula (VI) may be optionally further substituted with any convenient group, e.g., an alkyl group, a substituted alkyl group, a phenyl group, a substituted phenyl group, an ester group and the like. It will be understood that any convenient alternative hydrophilic group can be utilized as group X in a compound of formula (VI).

In some embodiments of formula (I), the structure has the formula (VII):

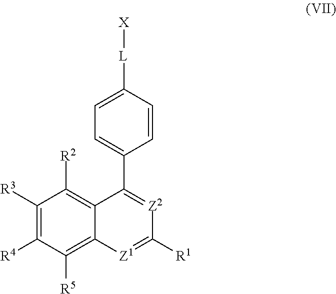

### Wherein,

L is selected from the group consisting of —CH₂—, —(CH₂)₂—, —(CH₂)₃—, —(CH₂)₄—, —(CH₂)₅— and —(CH₂)₆—;
X is selected from the group consisting of

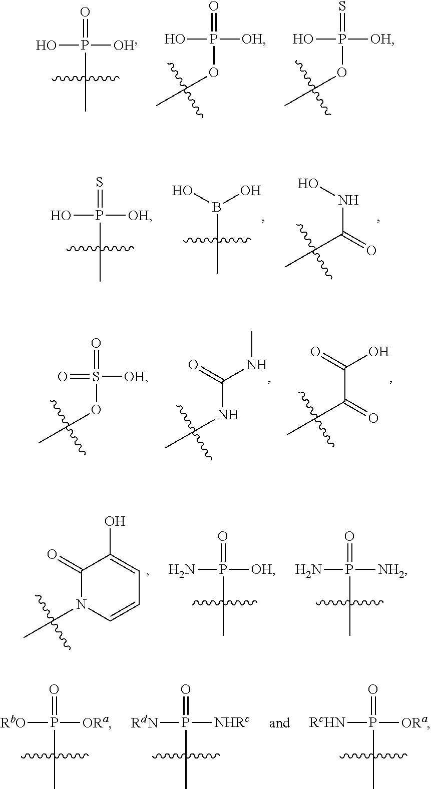

wherein R^{a} and R^{b} are each independently selected from aryl, alkyl, —CH₂OC(O)R^{e}, —CH₂OC(O)OR^{e}; R^{c} and R^{d} are each independently selected from —C(CH₃)C(O)ORe, alkyl and wherein R^{e} is alkyl;
Z¹ and Z² are each independently selected from C and N;
R¹ is selected from the group consisting of H, alkyl, substituted alkyl, alkenyl, substituted alkenyl, heterocycle and substituted heterocycle;
R² and R⁵ are each independently selected from the group consisting of H, OH, alkyl, substituted alkyl, alkenyl, substituted alkenyl, alkoxy, substituted alkoxy, —OCF₃, amine, substituted amine, amide, heterocycle and substituted heterocycle;
R³ and R⁴ are each independently selected from the group consisting of H, OH, alkyl, substituted alkyl, alkenyl, substituted alkenyl, alkoxy, substituted alkoxy, —OCF₃, amine, substituted amine, amide, heterocycle and substituted heterocycle;
or R³ and R⁴ together with the carbon to which they are attached form a group selected from heterocycle, substituted heterocycle, cycloalkyl, substituted cycloalkyl, aryl and substituted aryl,
or a pro-drug, a pharmaceutically acceptable salt or a solvate thereof.

In certain embodiments of formula (VII), at least one of Z¹ and Z² is N. In certain embodiments of formula (VII), Z¹ is C and Z² is N. In certain cases of formula (VII), Z¹ is N and Z² is C. In certain instances of formula (VII), Z¹ is C and Z² is C. In certain cases of formula (VII), Z¹ is N and Z² is N.

In some instances of formula (VII), R¹ is selected from hydrogen, C₁₋₅ alkyl, vinyl heterocycle (e.g., —CH═CH-heterocycle). In certain instances, the -vinyl heterocycle is vinyl pyridine (e.g., —CH═CH-pyridine). In some instances of formula (VII), R¹ is hydrogen. In some cases, R¹ is C₁₋₅ alkyl. In other cases R¹ is a vinyl heterocycle. In certain cases, R¹ is vinyl pyridine. In some instances, R² and R⁵ are both hydrogen. In some cases, R⁵ is selected from C₁₋₅ alkyl, amine, triazole, imidazole, amide, alkoxy, OCF₃ and hydroxy. In certain cases, R⁵ is alkoxy, e.g., methoxy. In some instances, R³ and R⁴ are each independently selected from hydrogen, C₁₋₅ alkyl, triazole, imidazole, amine, amide, alkoxy, OCF₃, hydroxy, or R³ and R⁴ together with the carbon to which they are attached from a heterocycle. In some cases, R³ and R⁴ are alkoxy, e.g., in some cases R³ and R⁴ are both methoxy. In some cases, R⁵ is methoxy and each of R¹-R⁴ are hydrogen. In some cases, R⁵ is methoxy, R¹ is —CH═CH-heterocycle and each of R²-R⁴ are hydrogen.

In certain embodiments of formula (VII), L is —CH₂—. In certain other cases of formula (VII), L is —(CH₂)₂—.

In certain embodiments of formula (VII), X is

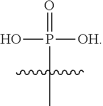 [SMILES: *P(=O)(O)O]

In certain cases of formula (VII), X is

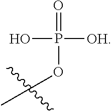 [SMILES: *OP(=O)(O)O]

In certain other cases of formula (VII), X is

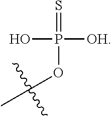 [SMILES: *OP(O)(O)=S]

In certain cases of formula (VII), X is

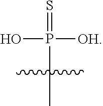 [SMILES: *P(O)(O)=S]

In certain other cases of formula (VII), X is

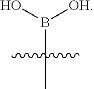 [SMILES: *B(O)O]

In certain embodiments of formula (VII), X is

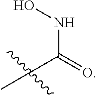 [SMILES: *C(=O)NO]

In certain cases of formula (VII), X is

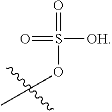 [SMILES: *OS(=O)(=O)O]

In certain other cases of formula (VII), X is

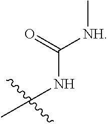 [SMILES: *NC(=O)NC]

In certain cases of formula (VII), X is

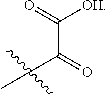 [SMILES: *C(=O)C(=O)O]

In certain other cases of formula (VII), X is

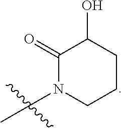 [SMILES: *N1CCCC(O)C1=O]

In certain cases of formula (VII), X is

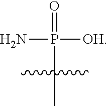 [SMILES: *P(N)(=O)O]

In certain other cases of formula (VII), X is

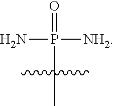 [SMILES: *P(N)(N)=O]

In certain other cases of formula (VI), X is

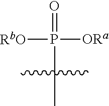

wherein R^{a} and R^{b} are each independently selected from aryl, alkyl, —CH₂OC(O)R^{e}, —CH₂OC(O)OR^{e}, wherein R^{e} is alkyl. In certain cases of formula (VI), X is

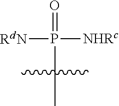

wherein R^{c} and R^{d} are each independently selected from —C(CH₃)C(O)Ore and alkyl, wherein R^{e} is alkyl. In certain other cases of formula (VI), X is

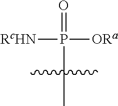

wherein R^{a} is selected from aryl, alkyl, —CH₂OC(O)R^{e}, —CH₂OC(O)OR^{e} and R^{c} is selected from —C(CH₃)C(O)Ore and alkyl, wherein R^{e} is alkyl.

It will be understood that any of the hydroxyl and amine groups in group X of formula (VII) may be optionally further substituted with any convenient group, e.g., an alkyl group, a substituted alkyl group, a phenyl group, a substituted phenyl group, an ester group and the like. It will be understood that any convenient alternative hydrophilic group can be utilized as group X in a compound of formula (VII).

In certain embodiments, the ENPP1 inhibitor is selected from the group consisting of

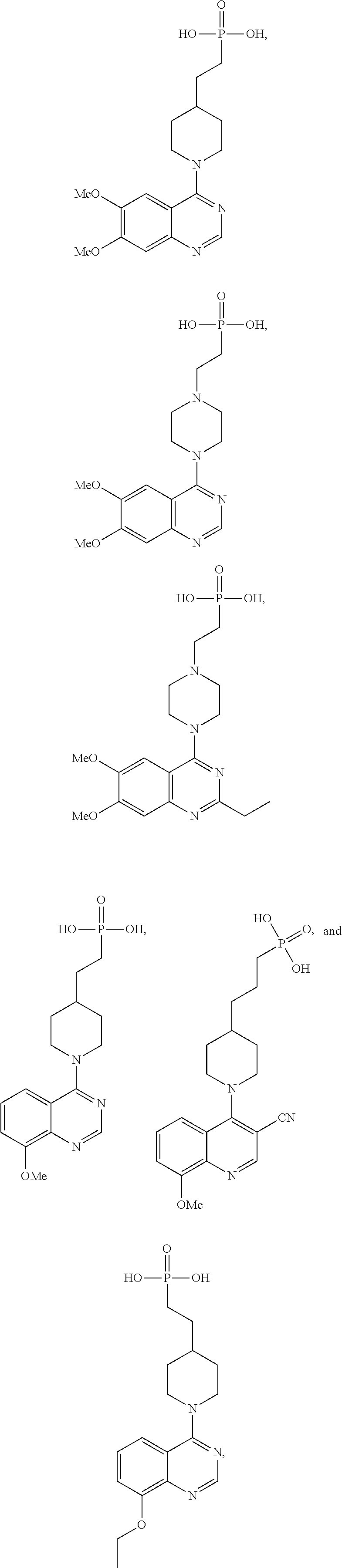

In certain embodiments, the compound is described by the structure of one of the compounds of Table 1 or Table 2.

TABLE 1
Compounds

| Cmpd | Structure |
|---|---|
| 1 | 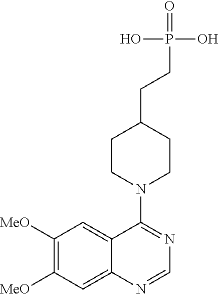 [SMILES: COc1cc2ncnc(N3CCC(CCP(=O)(O)O)CC3)c2cc1OC] |
| 2 | 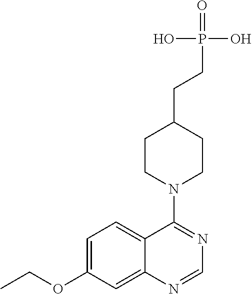 [SMILES: CCOc1ccc2c(N3CCC(CCP(=O)(O)O)CC3)ncnc2c1] |
| 3 | 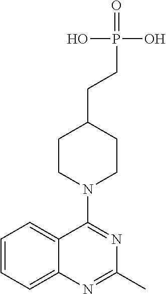 [SMILES: Cc1nc(N2CCC(CCP(=O)(O)O)CC2)c2ccccc2n1] |
| 4 | 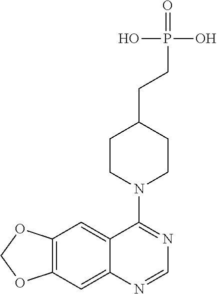 [SMILES: O=P(O)(O)CCC1CCN(c2ncnc3cc4c(cc23)OCO4)CC1] |
| 5 | 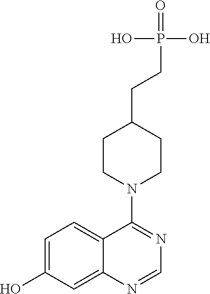 [SMILES: O=P(O)(O)CCC1CCN(c2ncnc3cc(O)ccc23)CC1] |
| 6 | 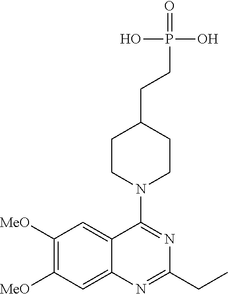 [SMILES: CCc1nc(N2CCC(CCP(=O)(O)O)CC2)c2cc(OC)c(OC)cc2n1] |
| 7 | 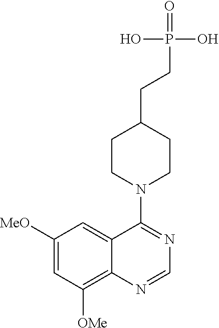 [SMILES: COc1cc(OC)c2ncnc(N3CCC(CCP(=O)(O)O)CC3)c2c1] |
| 8 | 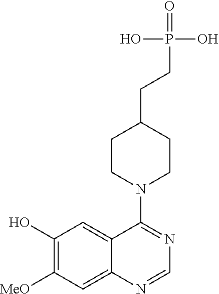 [SMILES: COc1cc2ncnc(N3CCC(CCP(=O)(O)O)CC3)c2cc1O] |
| 9 | 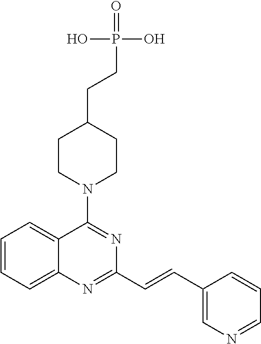 [SMILES: O=P(O)(O)CCC1CCN(c2nc(/C=C/c3cccnc3)nc3ccccc23)CC1] |
| 10 | 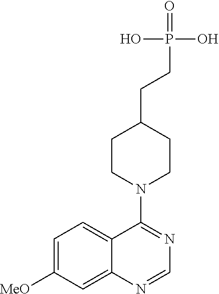 [SMILES: COc1ccc2c(N3CCC(CCP(=O)(O)O)CC3)ncnc2c1] |
| 11 | 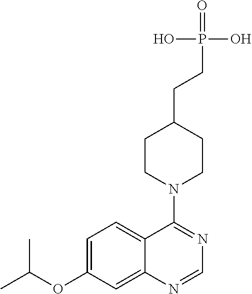 [SMILES: CC(C)Oc1ccc2c(N3CCC(CCP(=O)(O)O)CC3)ncnc2c1] |
| 12 | 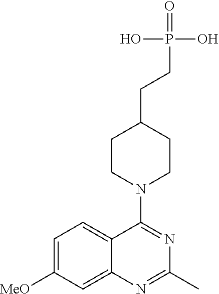 [SMILES: COc1ccc2c(N3CCC(CCP(=O)(O)O)CC3)nc(C)nc2c1] |
| 13 | 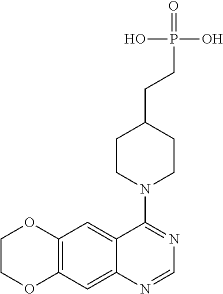 [SMILES: O=P(O)(O)CCC1CCN(c2ncnc3cc4c(cc23)OCCO4)CC1] |
| 14 | 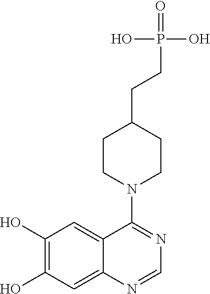 [SMILES: O=P(O)(O)CCC1CCN(c2ncnc3cc(O)c(O)cc23)CC1] |
| 15 | 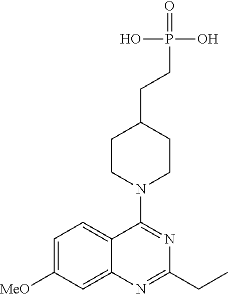 [SMILES: CCc1nc(N2CCC(CCP(=O)(O)O)CC2)c2ccc(OC)cc2n1] |
| 16 | 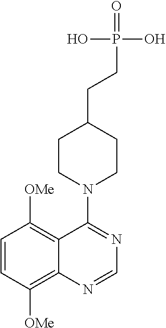 [SMILES: COc1ccc(OC)c2c(N3CCC(CCP(=O)(O)O)CC3)ncnc12] |
| 17 | 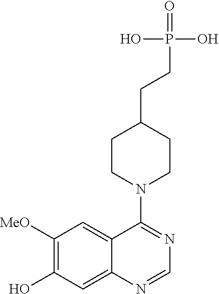 [SMILES: COc1cc2c(N3CCC(CCP(=O)(O)O)CC3)ncnc2cc1O] |
| 18 | 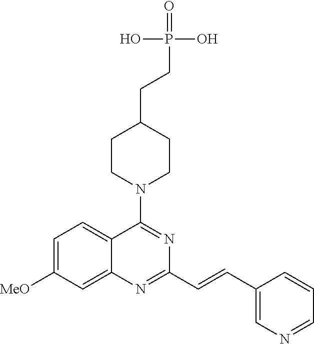 [SMILES: COc1ccc2c(N3CCC(CCP(=O)(O)O)CC3)nc(/C=C/c3cccnc3)nc2c1] |
| 19 | 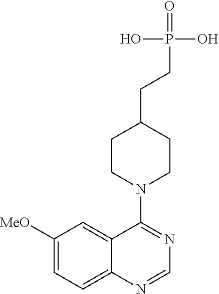 [SMILES: COc1ccc2ncnc(N3CCC(CCP(=O)(O)O)CC3)c2c1] |
| 20 | 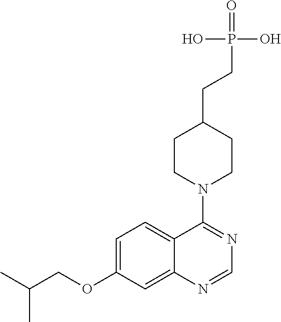 [SMILES: CC(C)COc1ccc2c(N3CCC(CCP(=O)(O)O)CC3)ncnc2c1] |
| 21 | 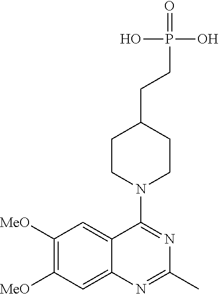 [SMILES: COc1cc2nc(C)nc(N3CCC(CCP(=O)(O)O)CC3)c2cc1OC] |
| 22 | 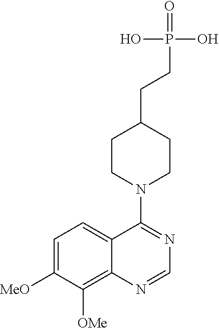 [SMILES: COc1ccc2c(N3CCC(CCP(=O)(O)O)CC3)ncnc2c1OC] |
| 23 | 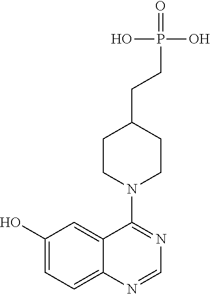 [SMILES: O=P(O)(O)CCC1CCN(c2ncnc3ccc(O)cc23)CC1] |
| 24 | 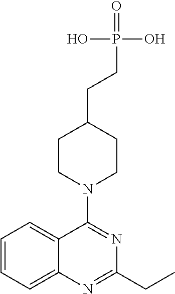 [SMILES: CCc1nc(N2CCC(CCP(=O)(O)O)CC2)c2ccccc2n1] |
| 25 | 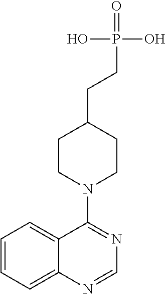 [SMILES: O=P(O)(O)CCC1CCN(c2ncnc3ccccc23)CC1] |
| 26 | 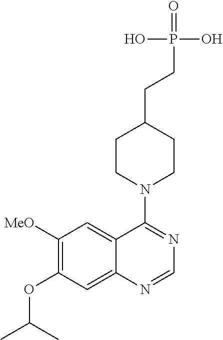 [SMILES: COc1cc2c(N3CCC(CCP(=O)(O)O)CC3)ncnc2cc1OC(C)C] |
| 27 | 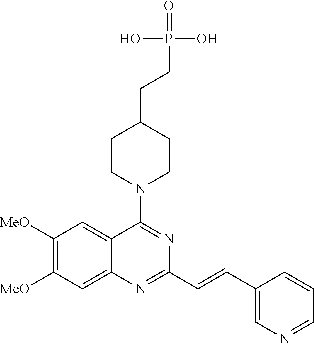 [SMILES: COc1cc2nc(/C=C/c3cccnc3)nc(N3CCC(CCP(=O)(O)O)CC3)c2cc1OC] |
| 28 | 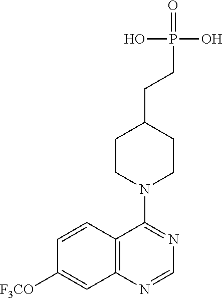 [SMILES: O=P(O)(O)CCC1CCN(c2ncnc3cc(OC(F)(F)F)ccc23)CC1] |
| 29 | 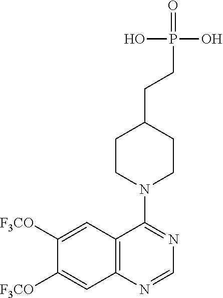 [SMILES: O=P(O)(O)CCC1CCN(c2ncnc3cc(OC(F)(F)F)c(OC(F)(F)F)cc23)CC1] |
| 30 | 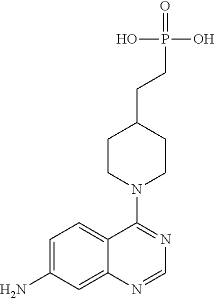 [SMILES: Nc1ccc2c(N3CCC(CCP(=O)(O)O)CC3)ncnc2c1] |
| 31 | 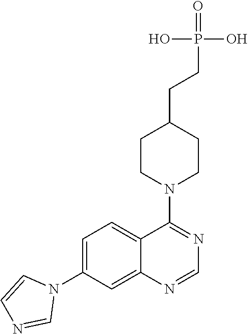 [SMILES: O=P(O)(O)CCC1CCN(c2ncnc3cc(-n4ccnc4)ccc23)CC1] |
| 32 | 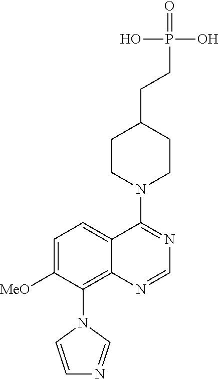 [SMILES: COc1ccc2c(N3CCC(CCP(=O)(O)O)CC3)ncnc2c1-n1ccnc1] |
| 33 | 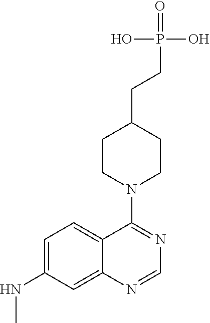 [SMILES: CNc1ccc2c(N3CCC(CCP(=O)(O)O)CC3)ncnc2c1] |
| 34 | 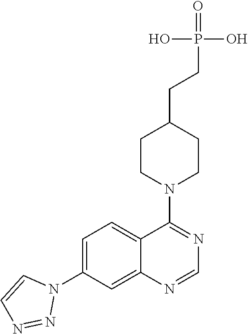 [SMILES: O=P(O)(O)CCC1CCN(c2ncnc3cc(-n4ccnn4)ccc23)CC1] |
| 35 | 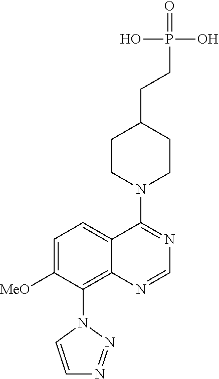 [SMILES: COc1ccc2c(N3CCC(CCP(=O)(O)O)CC3)ncnc2c1-n1ccnn1] |
| 36 | 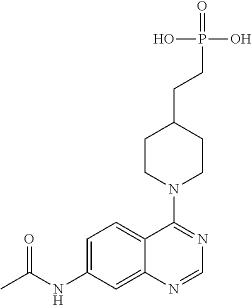 [SMILES: CC(=O)Nc1ccc2c(N3CCC(CCP(=O)(O)O)CC3)ncnc2c1] |
| 37 | 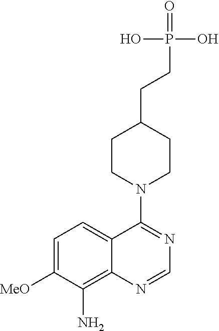 [SMILES: COc1ccc2c(N3CCC(CCP(=O)(O)O)CC3)ncnc2c1N] |
| 38 | 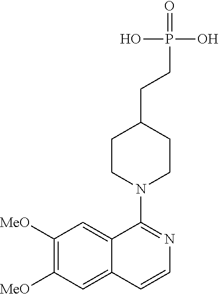 [SMILES: COc1cc2ccnc(N3CCC(CCP(=O)(O)O)CC3)c2cc1OC] |
| 39 | 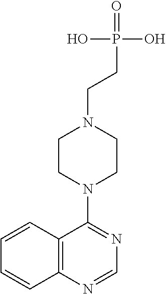 [SMILES: O=P(O)(O)CCN1CCN(c2ncnc3ccccc23)CC1] |
| 40 | 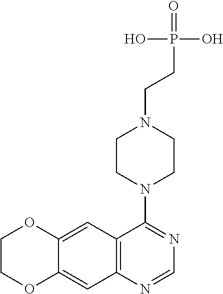 [SMILES: O=P(O)(O)CCN1CCN(c2ncnc3cc4c(cc23)OCCO4)CC1] |
| 41 | 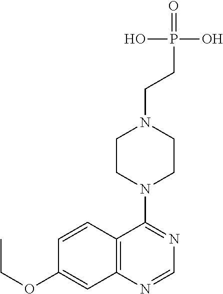 [SMILES: CCOc1ccc2c(N3CCN(CCP(=O)(O)O)CC3)ncnc2c1] |
| 42 | 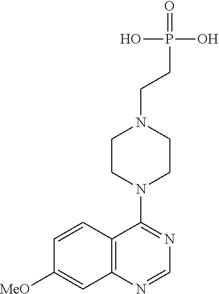 [SMILES: COc1ccc2c(N3CCN(CCP(=O)(O)O)CC3)ncnc2c1] |
| 43 | 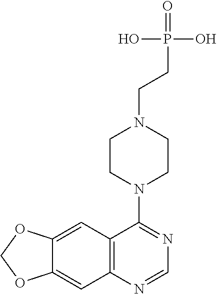 [SMILES: O=P(O)(O)CCN1CCN(c2ncnc3cc4c(cc23)OCO4)CC1] |
| 44 | 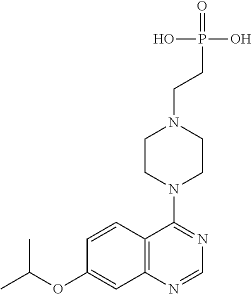 [SMILES: CC(C)Oc1ccc2c(N3CCN(CCP(=O)(O)O)CC3)ncnc2c1] |
| 45 | 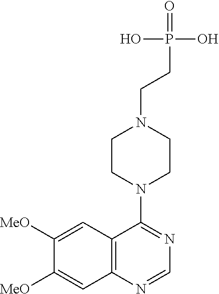 [SMILES: COc1cc2ncnc(N3CCN(CCP(=O)(O)O)CC3)c2cc1OC] |
| 46 | 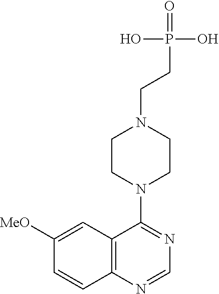 [SMILES: COc1ccc2ncnc(N3CCN(CCP(=O)(O)O)CC3)c2c1] |
| 47 | 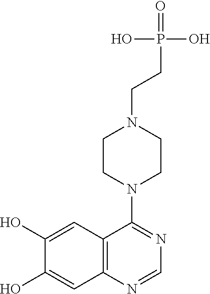 [SMILES: O=P(O)(O)CCN1CCN(c2ncnc3cc(O)c(O)cc23)CC1] |
| 48 | 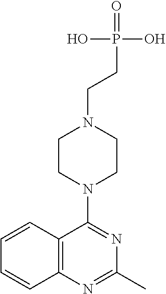 [SMILES: Cc1nc(N2CCN(CCP(=O)(O)O)CC2)c2ccccc2n1] |
| 49 | 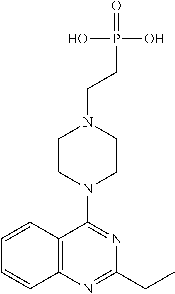 [SMILES: CCc1nc(N2CCN(CCP(=O)(O)O)CC2)c2ccccc2n1] |
| 50 | 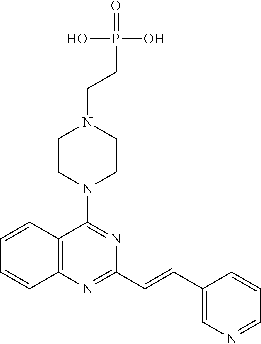 [SMILES: O=P(O)(O)CCN1CCN(c2nc(/C=C/c3cccnc3)nc3ccccc23)CC1] |
| 51 | 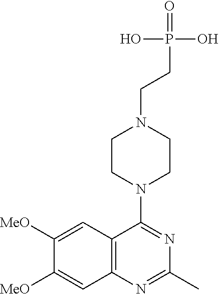 [SMILES: COc1cc2nc(C)nc(N3CCN(CCP(=O)(O)O)CC3)c2cc1OC] |
| 52 | 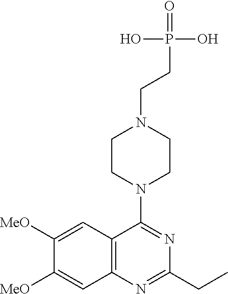 [SMILES: CCc1nc(N2CCN(CCP(=O)(O)O)CC2)c2cc(OC)c(OC)cc2n1] |
| 53 | 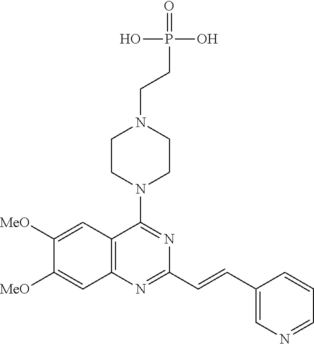 [SMILES: COc1cc2nc(/C=C/c3cccnc3)nc(N3CCN(CCP(=O)(O)O)CC3)c2cc1OC] |
| 54 | 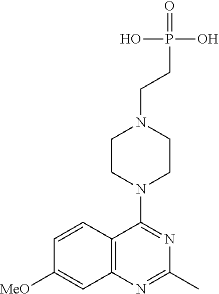 [SMILES: COc1ccc2c(N3CCN(CCP(=O)(O)O)CC3)nc(C)nc2c1] |
| 55 | 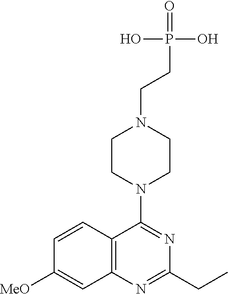 [SMILES: CCc1nc(N2CCN(CCP(=O)(O)O)CC2)c2ccc(OC)cc2n1] |
| 56 | 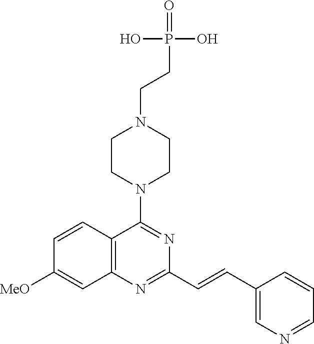 [SMILES: COc1ccc2c(N3CCN(CCP(=O)(O)O)CC3)nc(/C=C/c3cccnc3)nc2c1] |
| 57 | 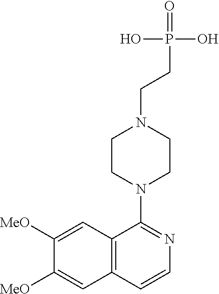 [SMILES: COc1cc2ccnc(N3CCN(CCP(=O)(O)O)CC3)c2cc1OC] |
| 58 | 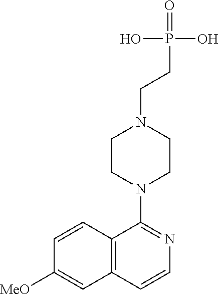 [SMILES: COc1ccc2c(N3CCN(CCP(=O)(O)O)CC3)nccc2c1] |
| 59 | 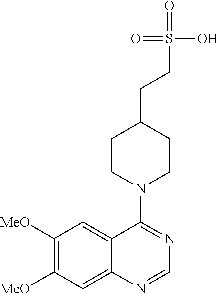 [SMILES: COc1cc2ncnc(N3CCC(CCS(=O)(=O)O)CC3)c2cc1OC] |
| 60 | 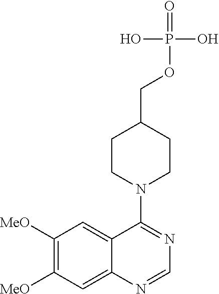 [SMILES: COc1cc2ncnc(N3CCC(COP(=O)(O)O)CC3)c2cc1OC] |
| 61 | 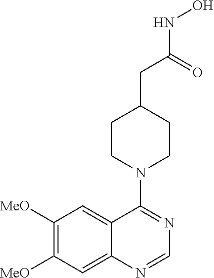 [SMILES: COc1cc2ncnc(N3CCC(CC(=O)NO)CC3)c2cc1OC] |
| 62 | 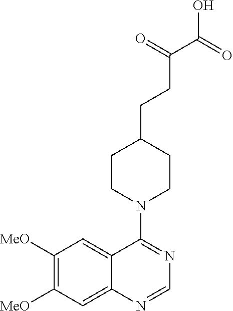 [SMILES: COc1cc2ncnc(N3CCC(CCC(=O)C(=O)O)CC3)c2cc1OC] |
| 63 | 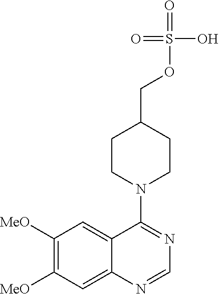 [SMILES: COc1cc2ncnc(N3CCC(COS(=O)(=O)O)CC3)c2cc1OC] |
| 64 | 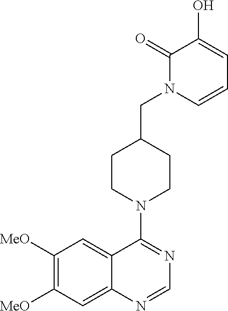 [SMILES: COc1cc2ncnc(N3CCC(Cn4cccc(O)c4=O)CC3)c2cc1OC] |
| 65 | 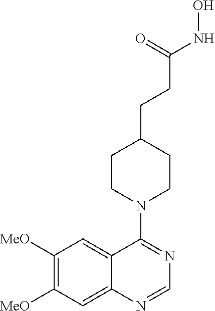 [SMILES: COc1cc2ncnc(N3CCC(CCC(=O)NO)CC3)c2cc1OC] |
| 66 | 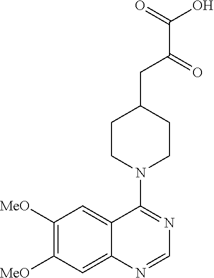 [SMILES: COc1cc2ncnc(N3CCC(CC(=O)C(=O)O)CC3)c2cc1OC] |
| 67 | 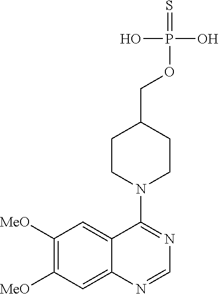 [SMILES: COc1cc2ncnc(N3CCC(COP(O)(O)=S)CC3)c2cc1OC] |
| 68 | 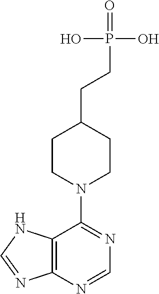 [SMILES: O=P(O)(O)CCC1CCN(c2ncnc3nc[nH]c23)CC1] |
| 69 | 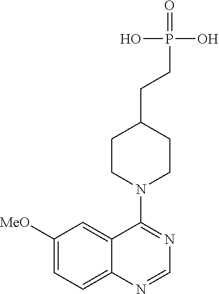 [SMILES: COc1ccc2ncnc(N3CCC(CCP(=O)(O)O)CC3)c2c1] |
| 70 | 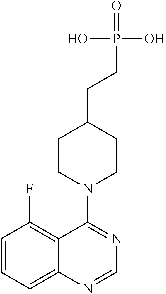 [SMILES: O=P(O)(O)CCC1CCN(c2ncnc3cccc(F)c23)CC1] |
| 71 | 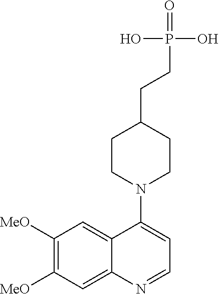 [SMILES: COc1cc2nccc(N3CCC(CCP(=O)(O)O)CC3)c2cc1OC] |
| 72 | 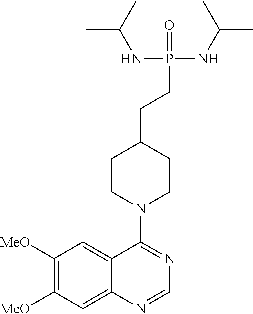 [SMILES: COc1cc2ncnc(N3CCC(CCP(=O)(NC(C)C)NC(C)C)CC3)c2cc1OC] |
| 73 | 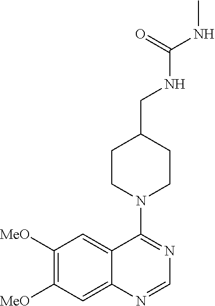 [SMILES: CNC(=O)NCC1CCN(c2ncnc3cc(OC)c(OC)cc23)CC1] |
| 74 | 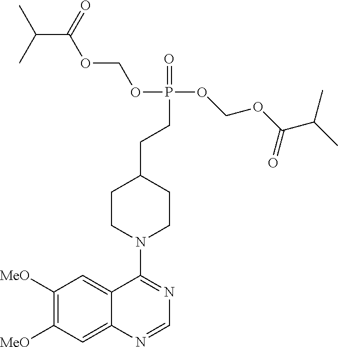 [SMILES: COc1cc2ncnc(N3CCC(CCP(=O)(OCOC(=O)C(C)C)OCOC(=O)C(C)C)CC3)c2cc1OC] |
| 75 | 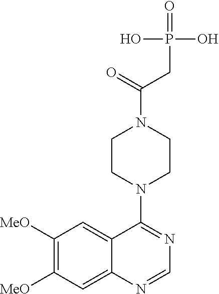 [SMILES: COc1cc2ncnc(N3CCN(C(=O)CP(=O)(O)O)CC3)c2cc1OC] |
| 76* | 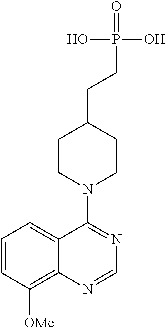 [SMILES: COc1cccc2c(N3CCC(CCP(=O)(O)O)CC3)ncnc12] |
| 77 | 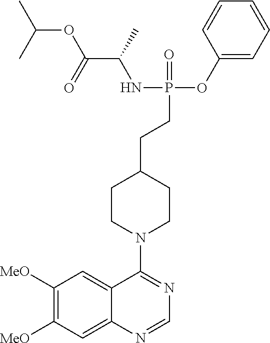 [SMILES: COc1cc2ncnc(N3CCC(CCP(=O)(N[C@@H](C)C(=O)OC(C)C)Oc4ccccc4)CC3)c2cc1OC] |
| 78 | 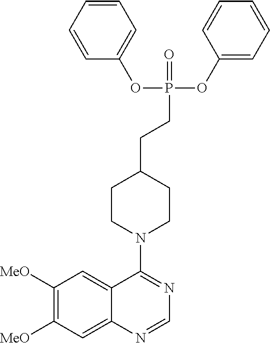 [SMILES: COc1cc2ncnc(N3CCC(CCP(=O)(Oc4ccccc4)Oc4ccccc4)CC3)c2cc1OC] |
| 79 | 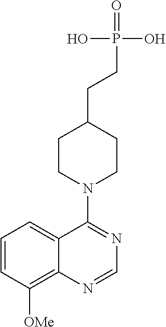 [SMILES: COc1cccc2c(N3CCC(CCP(=O)(O)O)CC3)ncnc12] |
| 80 | 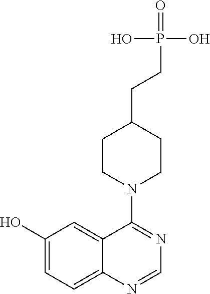 [SMILES: O=P(O)(O)CCC1CCN(c2ncnc3ccc(O)cc23)CC1] |
| 81 | 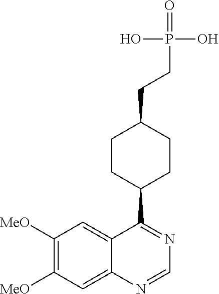 [SMILES: COc1cc2ncnc([C@H]3CC[C@@H](CCP(=O)(O)O)CC3)c2cc1OC] |
| 82 | 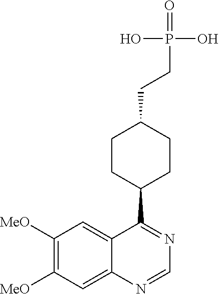 [SMILES: COc1cc2ncnc([C@H]3CC[C@H](CCP(=O)(O)O)CC3)c2cc1OC] |
| 83 | 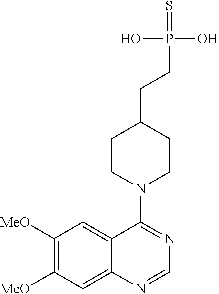 [SMILES: COc1cc2ncnc(N3CCC(CCP(O)(O)=S)CC3)c2cc1OC] |
| 84 | 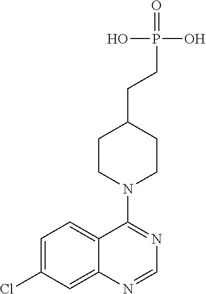 [SMILES: O=P(O)(O)CCC1CCN(c2ncnc3cc(Cl)ccc23)CC1] |
| 85 | 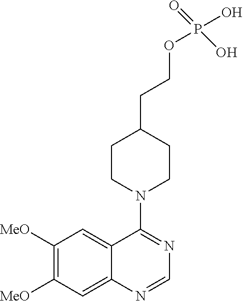 [SMILES: COc1cc2ncnc(N3CCC(CCOP(=O)(O)O)CC3)c2cc1OC] |
| 86 | 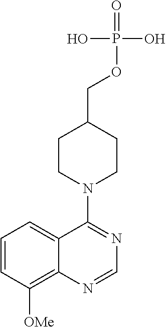 [SMILES: COc1cccc2c(N3CCC(COP(=O)(O)O)CC3)ncnc12] |
| 87 | 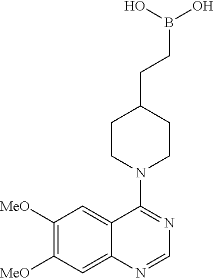 [SMILES: COc1cc2ncnc(N3CCC(CCB(O)O)CC3)c2cc1OC] |
| 88 | 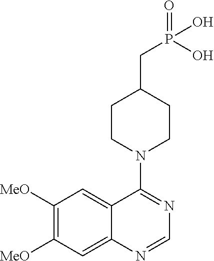 [SMILES: COc1cc2ncnc(N3CCC(CP(=O)(O)O)CC3)c2cc1OC] |
| 89 | 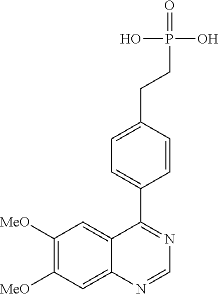 [SMILES: COc1cc2ncnc(-c3ccc(CCP(=O)(O)O)cc3)c2cc1OC] |
| 90 | 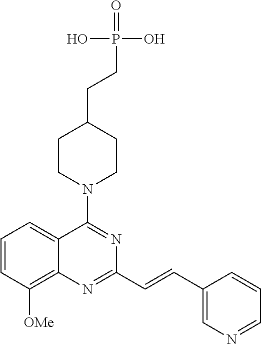 [SMILES: COc1cccc2c(N3CCC(CCP(=O)(O)O)CC3)nc(/C=C/c3cccnc3)nc12] |
| 91 | 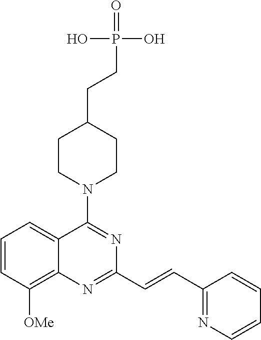 [SMILES: COc1cccc2c(N3CCC(CCP(=O)(O)O)CC3)nc(/C=C/c3ccccn3)nc12] |
| 92 | 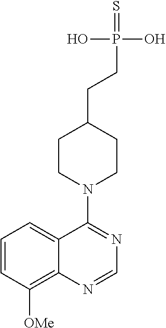 [SMILES: COc1cccc2c(N3CCC(CCP(O)(O)=S)CC3)ncnc12] |
| 201 | 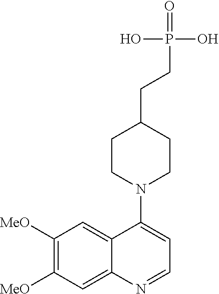 [SMILES: COc1cc2nccc(N3CCC(CCP(=O)(O)O)CC3)c2cc1OC] |
| 202 | 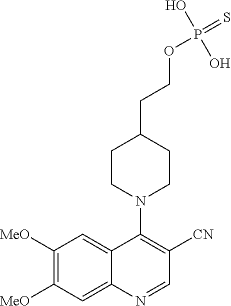 [SMILES: COc1cc2ncc(C#N)c(N3CCC(CCOP(O)(O)=S)CC3)c2cc1OC] |
| 203 | 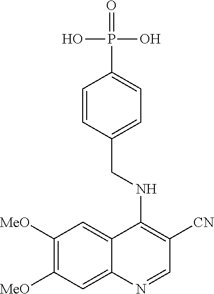 [SMILES: COc1cc2ncc(C#N)c(NCc3ccc(P(=O)(O)O)cc3)c2cc1OC] |
| 204 | 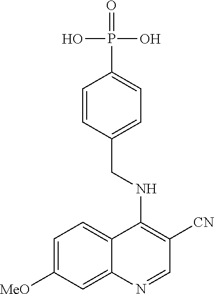 [SMILES: COc1ccc2c(NCc3ccc(P(=O)(O)O)cc3)c(C#N)cnc2c1] |
| 205 | 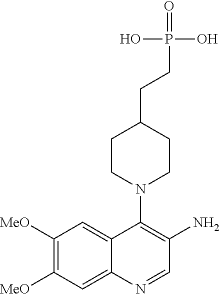 [SMILES: COc1cc2ncc(N)c(N3CCC(CCP(=O)(O)O)CC3)c2cc1OC] |
| 206 | 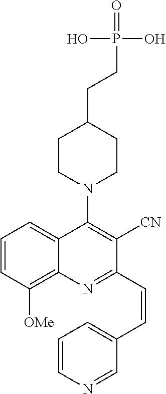 [SMILES: COc1cccc2c(N3CCC(CCP(=O)(O)O)CC3)c(C#N)c(/C=C\c3cccnc3)nc12] |
| 207 | 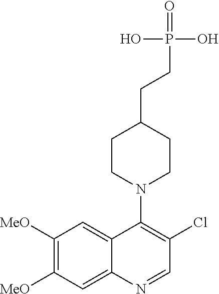 [SMILES: COc1cc2ncc(Cl)c(N3CCC(CCP(=O)(O)O)CC3)c2cc1OC] |
| 208 | 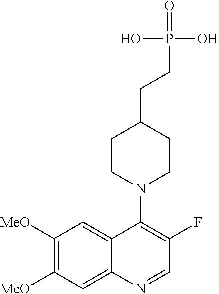 [SMILES: COc1cc2ncc(F)c(N3CCC(CCP(=O)(O)O)CC3)c2cc1OC] |
| 209 | 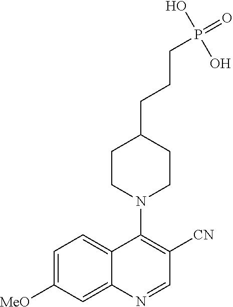 [SMILES: COc1ccc2c(N3CCC(CCCP(=O)(O)O)CC3)c(C#N)cnc2c1] |
| 210 | 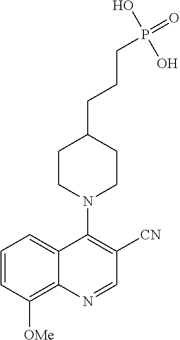 [SMILES: COc1cccc2c(N3CCC(CCCP(=O)(O)O)CC3)c(C#N)cnc12] |
| 211 | 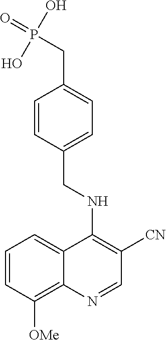 [SMILES: COc1cccc2c(NCc3ccc(CP(=O)(O)O)cc3)c(C#N)cnc12] |
| 212 | 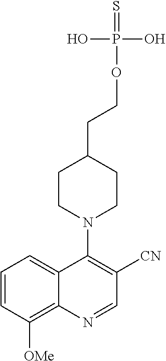 [SMILES: COc1cccc2c(N3CCC(CCOP(O)(O)=S)CC3)c(C#N)cnc12] |
| 213 | 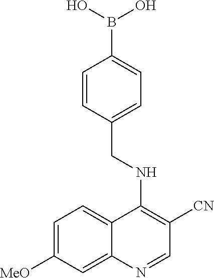 [SMILES: COc1ccc2c(NCc3ccc(B(O)O)cc3)c(C#N)cnc2c1] |
| 214 | 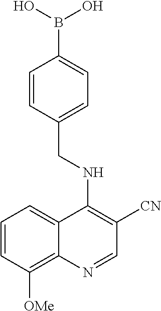 [SMILES: COc1cccc2c(NCc3ccc(B(O)O)cc3)c(C#N)cnc12] |
| 215 | 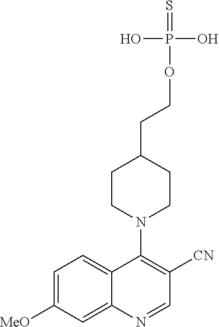 [SMILES: COc1ccc2c(N3CCC(CCOP(O)(O)=S)CC3)c(C#N)cnc2c1] |
| 216 | 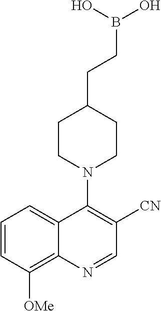 [SMILES: COc1cccc2c(N3CCC(CCB(O)O)CC3)c(C#N)cnc12] |
| 217 | 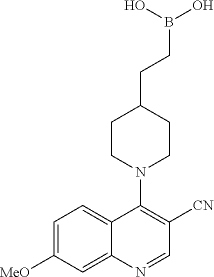 [SMILES: COc1ccc2c(N3CCC(CCB(O)O)CC3)c(C#N)cnc2c1] |
| 218 | 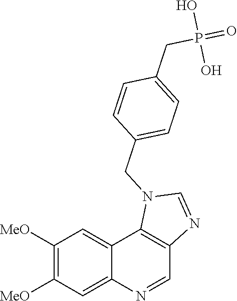 [SMILES: COc1cc2ncc3ncn(Cc4ccc(CP(=O)(O)O)cc4)c3c2cc1OC] |
| 219 | 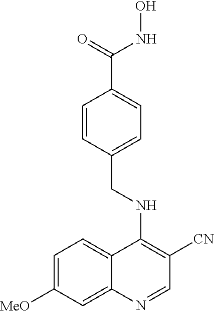 [SMILES: COc1ccc2c(NCc3ccc(C(=O)NO)cc3)c(C#N)cnc2c1] |
| 220 | 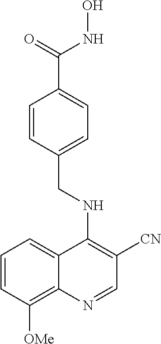 [SMILES: COc1cccc2c(NCc3ccc(C(=O)NO)cc3)c(C#N)cnc12] |
| 221 | 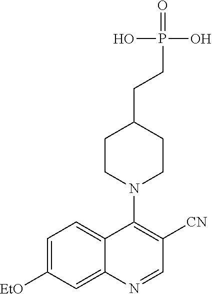 [SMILES: CCOc1ccc2c(N3CCC(CCP(=O)(O)O)CC3)c(C#N)cnc2c1] |
| 222 | 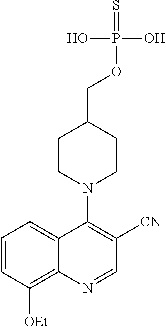 [SMILES: CCOc1cccc2c(N3CCC(COP(O)(O)=S)CC3)c(C#N)cnc12] |
| 226 | 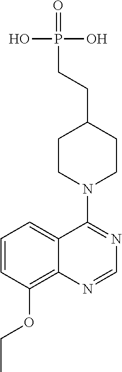 [SMILES: CCOc1cccc2c(N3CCC(CCP(=O)(O)O)CC3)ncnc12] |

*Compound 76 is also referred to as AG-3132 throughout the present disclosure.

TABLE 2
Compounds

| Cmpd | Structure |
|---|---|
| 93 | 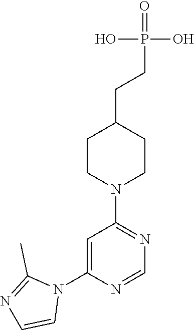 [SMILES: Cc1nccn1-c1cc(N2CCC(CCP(=O)(O)O)CC2)ncn1] |
| 94 | 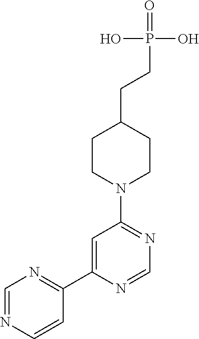 [SMILES: O=P(O)(O)CCC1CCN(c2cc(-c3ccncn3)ncn2)CC1] |
| 95 | 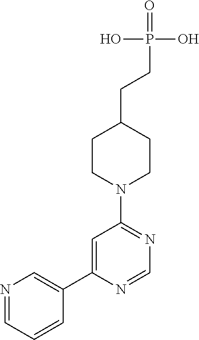 [SMILES: O=P(O)(O)CCC1CCN(c2cc(-c3cccnc3)ncn2)CC1] |
| 96 | 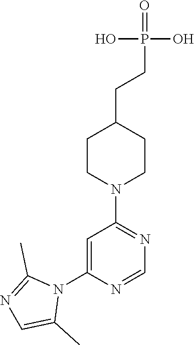 [SMILES: Cc1cnc(C)n1-c1cc(N2CCC(CCP(=O)(O)O)CC2)ncn1] |
| 97 | 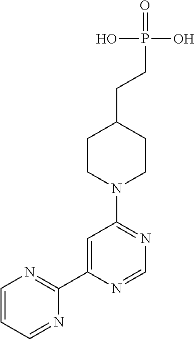 [SMILES: O=P(O)(O)CCC1CCN(c2cc(-c3ncccn3)ncn2)CC1] |
| 98 | 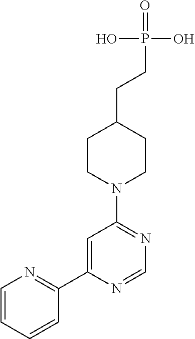 [SMILES: O=P(O)(O)CCC1CCN(c2cc(-c3ccccn3)ncn2)CC1] |
| 99 | 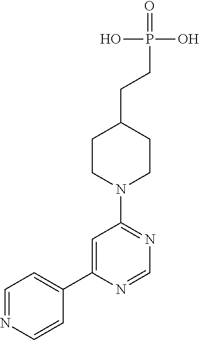 [SMILES: O=P(O)(O)CCC1CCN(c2cc(-c3ccncc3)ncn2)CC1] |
| 100 | 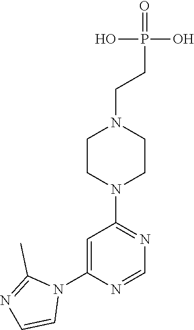 [SMILES: Cc1nccn1-c1cc(N2CCN(CCP(=O)(O)O)CC2)ncn1] |
| 101 | 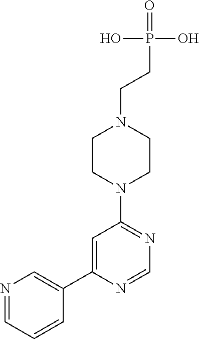 [SMILES: O=P(O)(O)CCN1CCN(c2cc(-c3cccnc3)ncn2)CC1] |
| 102 | 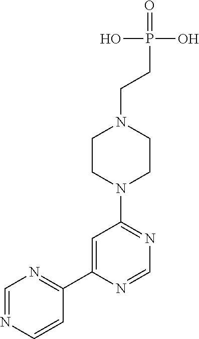 [SMILES: O=P(O)(O)CCN1CCN(c2cc(-c3ccncn3)ncn2)CC1] |
| 103 | 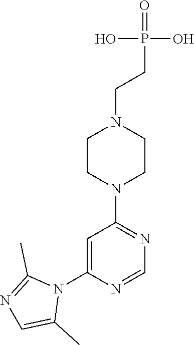 [SMILES: Cc1cnc(C)n1-c1cc(N2CCN(CCP(=O)(O)O)CC2)ncn1] |
| 104 | 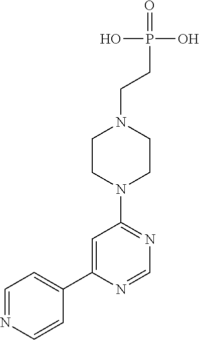 [SMILES: O=P(O)(O)CCN1CCN(c2cc(-c3ccncc3)ncn2)CC1] |
| 105 | 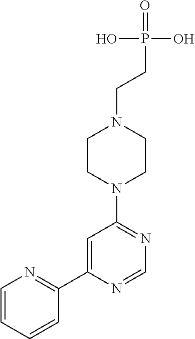 [SMILES: O=P(O)(O)CCN1CCN(c2cc(-c3ccccn3)ncn2)CC1] |
| 106 | 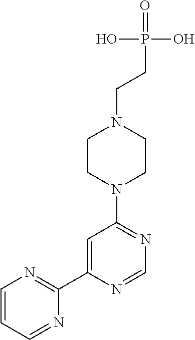 [SMILES: O=P(O)(O)CCN1CCN(c2cc(-c3ncccn3)ncn2)CC1] |

In certain embodiments, the compound is described by the structure of one of the compounds of Table 1 or Table 2. It is understood that any of the compounds shown in Table 1 or Table 2 may be present in a salt form. In some cases, the salt form of the compound is a pharmaceutically acceptable salt. It is understood that any of the compounds shown in Table 1 or Table 2 may be present in a prodrug form.

Aspects of the present disclosure include ENPP1 inhibitor compounds (e.g., as described herein), salts thereof (e.g., pharmaceutically acceptable salts), and/or solvate, hydrate and/or prodrug forms thereof. In addition, it is understood that, in any compound described herein having one or more chiral centers, if an absolute stereochemistry is not expressly indicated, then each center may independently be of R-configuration or S-configuration or a mixture thereof. It will be appreciated that all permutations of salts, solvates, hydrates, prodrugs and stereoisomers are meant to be encompassed by the present disclosure.

In some embodiments, the subject ENPP1 inhibitor compounds, or a prodrug form thereof, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the subject compounds are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)). In some cases, the promoiety is attached to a hydrophilic head group of the subject compounds. In some cases, the promoiety is attached to a hydroxy or carboxylic acid group of the subject compounds. In certain cases, the promoiety is an acyl or substituted acyl group. In certain cases, the promoiety is an alkyl or substituted alkyl group, e.g., that forms an ester functional group when attached to a hydrophilic head group of the subject compounds, e.g., a phosphonate ester, a phosphate ester, etc.

In some embodiments, the subject compounds, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, the subject compounds are provided by oral dosing and absorbed into the bloodstream. In some embodiments, the oral bioavailability of the subject compounds is 30% or more. Modifications may be made to the subject compounds or their formulations using any convenient methods to increase absorption across the gut lumen or their bioavailability.

In some embodiments, the subject compounds are metabolically stable (e.g., remain substantially intact in vivo during the half-life of the compound). In certain embodiments, the compounds have a half-life (e.g., an in vivo half-life) of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more.

In some embodiments, ENPP1 inhibitors include the formula:

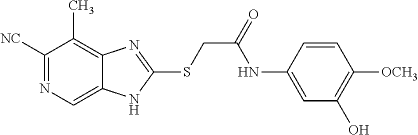 [SMILES: COc1ccc(NC(=O)CSc2nc3c(C)c(C#N)ncc3[nH]2)cc1O]

or a pharmaceutically acceptable salt thereof, additional details of which are described in US Application Pub. No. US20190031655A1, herein incorporated by reference for all purposes.

In some embodiments, ENPP1 inhibitors include the formula:

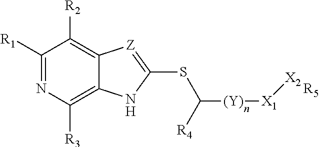

or a pharmaceutically acceptable salt thereof, additional details of which are described in US Application Pub. No. US20200039979A1, herein incorporated by reference for all purposes.

In some embodiments, ENPP1 inhibitors include the formula:

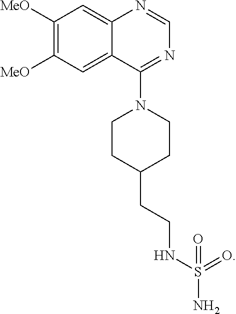 [SMILES: COc1cc2ncnc(N3CCC(CCNS(N)(=O)=O)CC3)c2cc1OC]

or a pharmaceutically acceptable salt thereof, additional details of which are described in International Application Pub. No. WO2018119328A1, herein incorporated by reference for all purposes.

In some embodiments, ENPP1 inhibitors include the formula:

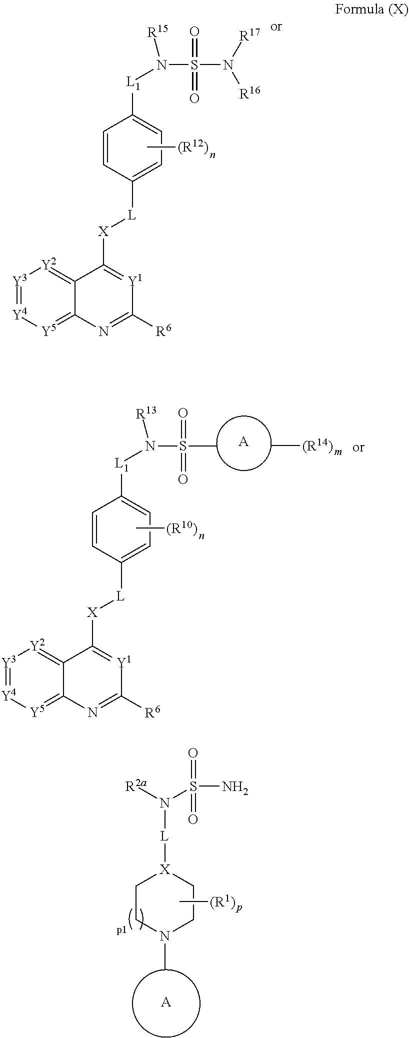

or pharmaceutically acceptable salts thereof, additional details of which are described in International Application Pub. No. WO2019046778A1 and US Application Pub. No. US20190282703A1, each herein incorporated by reference for all purposes.

In some embodiments, ENPP1 inhibitors include the formula:

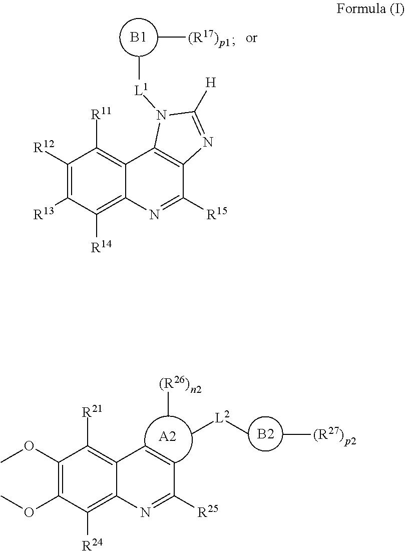

or pharmaceutically acceptable salts thereof, additional details of which are described in International Application Pub. No. WO2019177971A1, herein incorporated by reference for all purposes.

In some embodiments, ENPP1 inhibitors include the formula:

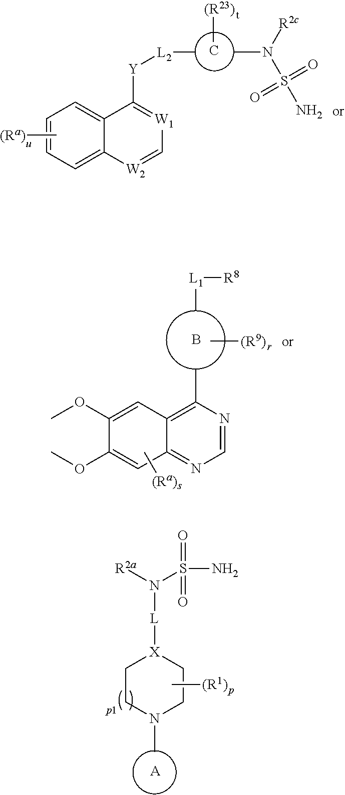

or pharmaceutically acceptable salts thereof, additional details of which are described in International Application Pub. No. WO2019191504A1, herein incorporated by reference for all purposes.

### Methods of Inhibiting ENPP1

As summarized above, aspects of the present disclosure include methods of use of ENPP1 inhibitors, and methods of inhibition using the same. ENPP1 is a member of the ecto-nucleotide pyrophosphatase/phosphodiesterase (ENPP) family. As such, aspects of the subject methods include inhibition of the hydrolase activity of ENPP1 against cGAMP. The inventors discovered that cGAMP can have significant extracellular biological functions, which can be enhanced by blocking extracellular degradation of cGAMP, e.g., hydrolysis by its degradation enzyme ENPP1. In certain instances, the ENPP1 target of inhibition is extracellular, and the subject ENPP1 inhibiting compounds are cell-impermeable, and thus are not capable of diffusion into cells. As such, the subject methods can provide for selective extracellular inhibition of ENPP1's hydrolase activity and increased extracellular levels of cGAMP. As such, in some cases, the ENPP1 inhibiting compounds are compounds that inhibit the activity of ENPP1 extracellularly. Experiments conducted by the inventors indicate that inhibiting the activity of ENPP1 increases extracellular cGAMP and may consequently boost the STING pathway.

By inhibiting a ENPP1 it is meant that the activity of the enzyme is decreased by 10% or more, such as 20% or more, 30% or more, 40% or more, 50% or more, 60% or more, 70% or more, 80% or more, 90% or more, 95% or more (e.g., relative to a control in any convenient in vitro inhibition assay). In some cases, inhibiting a ENPP1 means decreasing the activity of the enzyme by a factor of 2 or more, such as 3 or more, 5 or more, 10 or more, 100 or more, or 1000 or more, relative to its normal activity (e.g., relative to a control as measured by any convenient assay).

In some cases, the method is a method of inhibiting ENPP1 in a sample. The term “sample” as used herein relates to a material or mixture of materials, typically, although not necessarily, in fluid form, containing one or more components of interest.

In some embodiments, there is provided a method of inhibiting ENPP1, the method comprising contacting a sample with a cell impermeable ENPP1 inhibitor to inhibit cGAMP hydrolysis activity of ENPP1. In some cases, the sample is a cellular sample. In some cases, the sample comprises cGAMP. In certain cases, the cGAMP levels are elevated in the cellular sample (e.g., relative to a control sample not contacted with the inhibitor). The subject methods can provide for increased levels of cGAMP. By “increased level of cGAMP” is meant a level of cGAMP in a cellular sample contacted with a subject compound, where the cGAMP level in the sample is increased by 10% or more, such as 20% or more, 30% or more, 40% or more, 50% or more, 60% or more, 70% or more, 80% or more, 90% or more, 100% or more, or even more, relative to a control sample that is not contacted with the agent.

In certain embodiments the cell impermeable ENPP1 inhibitor is an inhibitor as defined herein. In some embodiments, the cell impermeable ENPP1 inhibitor is an inhibitor according to any one of formulas I, IV V, VI or VII. In some cases, the cell impermeable ENPP1 inhibitor is any one of compounds 1-106.

In some embodiments the ENPP1 inhibitor is cell permeable. In some embodiments, there is provided a method of inhibiting ENPP1, the method comprising contacting a sample with a cell permeable ENPP1 inhibitor to inhibit ENPP1.

In some embodiments, the subject compounds have an ENPP1 inhibition profile that reflects activity against additional enzymes. In some embodiments, the subject compounds specifically inhibit ENPP1 without undesired inhibition of one or more other enzymes.

In some embodiments, the compounds of the disclosure interfere with the interaction of cGAMP and ENPP1. For example, the subject compounds may act to increase the extracellular cGAMP by inhibiting the hydrolase activity of ENPP1 against cGAMP. Without being bound to any particular theory, it is thought that increasing extracellular cGAMP activates the STING pathway.

In some embodiments, the subject compounds inhibit ENPP1, as determined by an inhibition assay, e.g., by an assay that determines the level of activity of the enzyme either in a cell-free system or in a cell after treatment with a subject compound, relative to a control, by measuring the IC₅₀ or EC₅₀ value, respectively. In certain embodiments, the subject compounds have an IC₅₀ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower.

As summarized above, aspects of the disclosure include methods of inhibiting ENPP1. A subject compound (e.g., as described herein) may inhibit at activity of ENPP1 in the range of 10% to 100%, e.g., by 10% or more, 20% or more, 30% or more, 40% or more, 50% or more, 60% or more, 70% or more, 80% or more, or 90% or more. In certain assays, a subject compound may inhibit its target with an IC₅₀ of 1×10⁻⁶ M or less (e.g., 1×10⁻⁶ M or less, 1×10⁻⁷ M or less, 1×10⁻⁸ M or less, 1×10⁻⁹ M or less, 1×10⁻¹⁰ M or less, or 1×10⁻¹¹ M or less).

The protocols that may be employed in determining ENPP1 activity are numerous, and include but are not limited to cell-free assays, e.g., binding assays; assays using purified enzymes, cellular assays in which a cellular phenotype is measured, e.g., gene expression assays; and in vivo assays that involve a particular animal (which, in certain embodiments may be an animal model for a condition related to the target pathogen).

In some embodiments, the subject method is an in vitro method that includes contacting a sample with a subject compound that specifically inhibits ENPP1. In certain embodiments, the sample is suspected of containing ENPP1 and the subject method further comprises evaluating whether the compound inhibits ENPP1.

In certain embodiments, the subject compound is a modified compound that includes a label, e.g., a fluorescent label, and the subject method further includes detecting the label, if present, in the sample, e.g., using optical detection.

In certain embodiments, the compound is modified with a support or with affinity groups that bind to a support (e.g. biotin), such that any sample that does not bind to the compound may be removed (e.g., by washing). The specifically bound ENPP1, if present, may then be detected using any convenient means, such as, using the binding of a labeled target specific probe, or using a fluorescent protein reactive reagent.

In another embodiment of the subject method, the sample is known to contain ENPP1.

In some embodiments, the method is a method of reducing cancer cell proliferation, where the method includes contacting the cell with an effective amount of a subject ENPP1 inhibitor compound (e.g., as described herein) to reduce cancer cell proliferation. In some embodiments, the subject ENPP1 inhibitor compounds can act extracellularly. In certain cases, the subject ENPP1 inhibitor compounds can act intracellularly. The method can be performed in combination with a chemotherapeutic agent (e.g., as described herein). The cancer cells can be in vitro or in vivo. In certain instances, the method includes contacting the cell with an ENPP1 inhibitor compound (e.g., as described herein) and contacting the cell with a chemotherapeutic agent. Any convenient cancer cells can be targeted.

### Immune Checkpoint Inhibitors

The immune checkpoint inhibitor used in the methods disclosed herein can be any one of a plurality of molecules effective as immune checkpoint inhibitors.

In some aspects, the immune checkpoint inhibitor is a cytotoxic T-lymphocyte-associated antigen 4 (CTLA-4) inhibitor, a programmed death 1 (PD-1) inhibitor, or a PD-L1 inhibitor.

In some aspects, the immune checkpoint inhibitor is atezolizumab, avelumab, durvalumab, cemiplimab, ipilimumab, pembrolizumab, nivolumab, LAG525 (IMP701), REGN3767 (R3767), BI 754,091, tebotelimab (MGD013), eftilagimod alpha (IMP321), FS118, MBG453, Sym023, TSR-022, MGC018, FPA150, EOS100850, AB928, CPI-006, Monalizumab, COM701, CM24, NEO-201, Defactinib, PF-04136309, MSC-1, Hu5F9-G4 (5F9), ALX148, TTI-662, RRx-001, Lacnotuzumab (MCS110), LY3022855, SNDX-6352, emactuzumab (RG7155), pexidartinib (PLX3397), CAN04, Canakinumab (ACZ885), BMS-986253, Pepinemab (VX15/2503), Trebananib, FP-1305, Enapotamab vedotin (EnaV) or baviuximab.

In some aspects, the immune check point inhibitor is atezolizumab, avelumab, durvalumab, cemiplimab, ipilimumab, pembrolizumab, or nivolumab. In some aspects, the immune check point inhibitor is ipilimumab, pembrolizumab, or nivolumab.

### Methods of Treatment

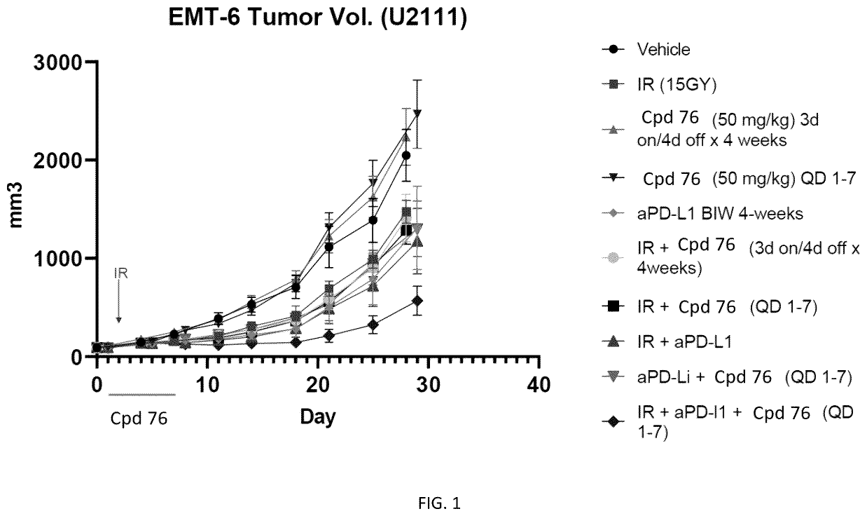

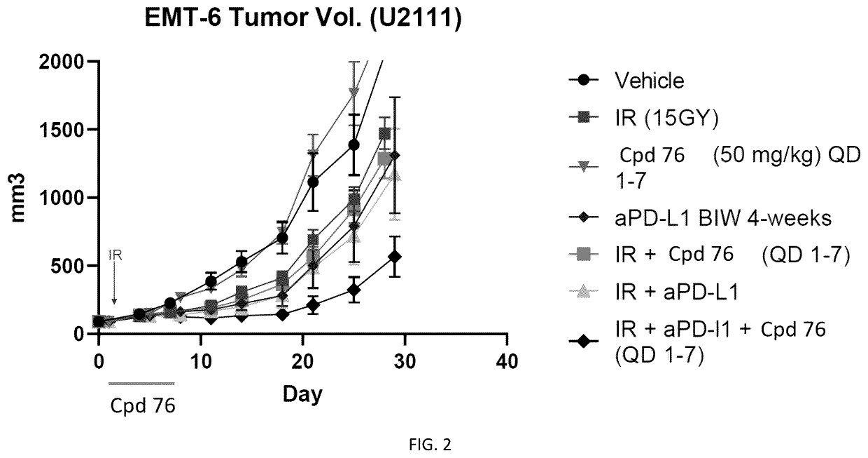

Aspects of the present disclosure include methods for inhibiting the hydrolase activity of ENPP1 against cGAMP provides for increased levels of cGAMP and/or downstream modulation (e.g., activation) of the STING pathway. The inventors have discovered that cGAMP is present in the extracellular space and that ENPP1 can control extracellular levels of cGAMP. The inventors have also discovered that cGAMP can have significant extracellular biological functions in vivo (e.g. see FIG. 1-2). The results described and demonstrated herein indicate that ENPP1 inhibition according to the subject methods can modulate STING activity in vivo, and thus find use in the treatment of a variety of diseases, e.g., as a target for cancer immunotherapy. As such, the subject methods provide for selective extracellular inhibition of ENPP1 activity (e.g., hydrolase activity of cGAMP) to increase extracellular levels of cGAMP and activate the stimulator of interferon genes (STING) pathway. In some instances, the subject method is a method for increasing a STING mediated response in a subject. In some instances, the subject method is a method for modulating an immune response in a subject.

A “STING mediated response” refers to any response that is mediated by STING, including, but not limited to, immune responses, e.g., to bacterial pathogens, viral pathogens, and eukaryotic pathogens. See, e.g., Ishikawa et al. Immunity 29: 538-550 (2008); Ishikawa et al. Nature 461: 788-792 (2009); and Sharma et al. Immunity 35: 194-207 (2011). STING also functions in certain autoimmune diseases initiated by inappropriate recognition of self DNA (see, e.g., Gall et al. Immunity 36: 120-131 (2012), as well as for the induction of adaptive immunity in response to DNA vaccines (see, e.g., Ishikawa et al. Nature 461: 788-792 (2009). By increasing a STING mediated response in a subject is meant an increase in a STING mediated response in a subject as compared to a control subject (e.g., a subject who is not administered a subject compound). In some cases, the subject is human and the subject compounds and methods provide for activation of human STING. In some cases, the STING mediated response includes modulation of an immune response. In some instances, the subject method is a method of modulating an immune response in a subject.

In some cases, the STING mediated response includes increasing the production of an interferon (e.g., a type I interferon (IFN), type III interferon (IFN)) in a subject. Interferons (IFNs) are proteins having a variety of biological activities, e.g., antiviral, immunomodulating and antiproliferative. IFNs are relatively small, species-specific, single chain polypeptides, produced by mammalian cells in response to exposure to a variety of inducers such as viruses, polypeptides, mitogens and the like. Interferons protect animal tissues and cells against viral attack and are an important host defense mechanism. Interferons may be classified as Type-I, Type-II and Type-III interferons. Mammalian Type-I interferons of interest include IFN-α (alpha), IFN-β (beta), IFN-γ (gamma), IFN-κ (kappa), IFN-δ (delta), IFN-ε (epsilon), IFN-τ (tau), IFN-ω (omega), and IFN-ζ (zeta, also known as limiting).

Interferons find use in the treatment of a variety of cancers since these molecules have anti-cancer activity that acts at multiple levels. Interferon proteins can directly inhibit the proliferation of human tumor cells. In some cases, the anti-proliferative activity is also synergistic with a variety of approved chemotherapeutic agents such as cisplatin, 5FU and paclitaxel. The immunomodulatory activity of interferon proteins can also lead to the induction of an anti-tumor immune response. This response includes activation of NK cells, stimulation of macrophage activity and induction of MHC class I surface expression, leading to the induction of anti-tumor cytotoxic T lymphocyte activity. In addition, interferons play a role in cross-presentation of antigens in the immune system. Moreover, some studies further indicate that IFN-β protein may have anti-angiogenic activity. Angiogenesis, new blood vessel formation, is critical for the growth of solid tumors. IFN-β may inhibit angiogenesis by inhibiting the expression of pro-angiogenic factors such as bFGF and VEGF. Interferon proteins may also inhibit tumor invasiveness by modulating the expression of enzymes, such as collagenase and elastase, which are important in tissue remodeling.

Aspects of the methods include administering to a subject an effective amount of an ENPP1 inhibitor to treat or prevent metastasis of cancer in the subject. Any convenient ENPP1 inhibitors can be used in the subject methods of treating cancer. In certain cases, the ENPP1 inhibitor compound is a compound as described herein. In certain cases, the ENPP1 inhibitor is a cell impermeable compound. In certain cases, the ENPP1 inhibitor is a cell permeable compound. In certain cases the cancer is a solid cancer e.g. a lymphoma. In certain embodiments, the cancer is selected from adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioblastomas, melanoma and various head and neck tumors.

Aspects of the methods include administering to a subject an effective amount of a cell impermeable ENPP1 inhibitor to inhibit the hydrolysis of cGAMP and treat or prevent metastasis of cancer in the subject. In certain cases the cancer is a solid cancer e.g. a lymphoma. In certain embodiments, the cancer is selected from adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioblastomas, melanoma and various head and neck tumors.

In some embodiments, the subject is to undergo or has undergone resection of a malignant tumor. The resection may have occurred immediately before, or days, weeks, months, or years before use of the methods herein.

In some embodiments, the method comprises suppressing or preventing the recurrence of the cancer. This suppression could be for days, weeks, months, or years.

In some embodiments, the method comprises suppressing or preventing malignant tumor cells from colonizing or invading vascular tissue.

In some embodiments of the methods disclosed herein, the cell impermeable ENPP1 inhibitor is an inhibitor of any one of formulas I, IV, V, VI or VII. In some cases, the cell impermeable ENPP1 inhibitor is any one of compounds 1-106, 201-222, and 226.

In some embodiments of the methods disclosed herein, the ENPP1 inhibitor is cell permeable.

As such, aspects of the method include contacting a sample with a subject compound (e.g., as described above) under conditions by which the compound inhibits ENPP1. Any convenient protocol for contacting the compound with the sample may be employed. The particular protocol that is employed may vary, e.g., depending on whether the sample is in vitro or in vivo. For in vitro protocols, contact of the sample with the compound may be achieved using any convenient protocol. In some instances, the sample includes cells that are maintained in a suitable culture medium, and the complex is introduced into the culture medium. For in vivo protocols, any convenient administration protocol may be employed. Depending upon the potency of the compound, the cells of interest, the manner of administration, the number of cells present, various protocols may be employed.

In some embodiments, the subject method includes administering to the subject an effective amount of a subject compound (e.g., as described herein) or a pharmaceutically acceptable salt thereof. The subject compound may be administered as part of a pharmaceutical composition (e.g., as described herein). In certain instances of the method, the compound that is administered is a compound of one of formulae (I), (IV), (V), (VI) or (VII). In certain instances of the method, the compound that is administered is described by one of the compounds of Table 1 or 2.

In some embodiments, an “effective amount” is an amount of a subject compound that, when administered to an individual in one or more doses, in monotherapy or in combination therapy, is effective to inhibit ENPP1 by about 20% (20% inhibition), at least about 30% (30% inhibition), at least about 40% (40% inhibition), at least about 50% (50% inhibition), at least about 60% (60% inhibition), at least about 70% (70% inhibition), at least about 80% (80% inhibition), or at least about 90% (90% inhibition), compared to the ENPP1 activity in the individual in the absence of treatment with the compound, or alternatively, compared to the ENPP1 activity in the individual before or after treatment with the compound.

In some embodiments, an “effective amount” is an amount of a subject compound that, when administered to an individual in one or more doses, in monotherapy or in combination therapy, is effective to decrease metastasis in the subject by about 20%, at least about 30%, at least about 40%, at least about 50%, at least about 60%, at least about 70%, at least about 80%, or at least about 90%, compared to metastasis in the individual in the absence of treatment with the compound, or alternatively, reducing the tumor burden load (or number of tumors) compared to the tumor burden load (or number of tumors) in the subject before or after treatment with the compound.

In some embodiments, an “effective amount” is an amount of a subject compound that, when administered to an individual in one or more doses, in monotherapy or in combination therapy, is effective to reduce the dose of radiotherapy required to observe reduction in metastasis in the subject by about 20%, at least about 30%, at least about 40%, at least about 50%, at least about 60%, at least about 70%, at least about 80%, or at least about 90%, compared to the dose of radiotherapy required to observe reduced metastasis in the individual in the absence of treatment with the compound.

In some embodiments, an “effective amount” is an amount of a subject compound that, when administered to an individual in one or more doses, in monotherapy or in combination therapy, is effective to increase the progression-free survival of the subject by about 20%, at least about 30%, at least about 40%, at least about 50%, at least about 60%, at least about 70%, at least about 80%, at least about 90%, at least about 100%, or more than 100% compared to the progression-free survival of the individual in the absence of treatment with the compound.

In some embodiments, an “effective amount” of a compound is an amount that, when administered in one or more doses to an individual having cancer, is effective to achieve a 1.5-log, a 2-log, a 2.5-log, a 3-log, a 3.5-log, a 4-log, a 4.5-log, or a 5-log reduction in tumor size.

In some embodiments, an effective amount of a compound is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of a compound is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of a compound is administered. In other embodiments, multiple doses are administered. Where multiple doses are administered over a period of time, the compound can be administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

Any of a variety of methods can be used to determine whether a treatment method is effective. For example, a biological sample obtained from an individual who has been treated with a subject method can be assayed.

Any of the compounds described herein can be utilized in the subject methods of treatment. In certain instances, the compound is of one of formulae I, IV or V. In certain cases, the compound is one of the compounds of Table 1 or 2. In some cases, the compound that is utilized in the subject methods is not cell permeable. In some cases, the compound that is utilized in the subject methods has poor cell permeability.

In some embodiments, the compound specifically inhibits ENPP1. In some embodiments, the compound modulates the activity of cGAMP. In some embodiments, the compound interferes with the interaction of ENPP1 and cGAMP. In some embodiments, the compound results in activation of the STING pathway.

In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). The subject may be in need of treatment for cancer. In some instances, the subject methods include diagnosing cancer, including any one of the cancers described herein. In some embodiments, the compound is administered as a pharmaceutical preparation.

In certain embodiments, the ENPP1 inhibitor compound is a modified compound that includes a label, and the method further includes detecting the label in the subject. The selection of the label depends on the means of detection. Any convenient labeling and detection systems may be used in the subject methods, see e.g., Baker, “The whole picture,” Nature, 463, 2010, p 977-980. In certain embodiments, the compound includes a fluorescent label suitable for optical detection. In certain embodiments, the compound includes a radiolabel for detection using positron emission tomography (PET) or single photon emission computed tomography (SPECT). In some cases, the compound includes a paramagnetic label suitable for tomographic detection. The subject compound may be labeled, as described above, although in some methods, the compound is unlabeled and a secondary labeling agent is used for imaging.

### Combination Therapies

The subject compounds can be administered to a subject alone or in combination with one or more additional, i.e., second, third, etc. active agent. Combination therapeutic methods where the subject ENPP1 inhibitor compounds may be used in combination with a second active agent or an additional therapy, e.g., radiation therapy. The terms “agent,” “compound,” and “drug” are used interchangeably herein. For example, ENPP1 inhibitor compounds can be administered alone or in conjunction with one or more other drugs, such as drugs employed in the treatment of diseases of interest, including but not limited to, immunomodulatory diseases and conditions and cancer. In some embodiments, the subject method further includes coadministering concomitantly or in sequence a second agent, e.g., a small molecule, a chemotherapeutic, an antibody, an antibody fragment, an antibody-drug conjugate, an aptamer, a protein, or a checkpoint inhibitor. In some embodiments, the method further includes performing radiation therapy on the subject. In some embodiments, the radiation therapy is with ionizing radiation (IR).

In some embodiments, the method further includes performing adoptive cell therapy on the subject. In certain aspects, use of adoptive immunotherapy in a subject in need of treatment in combination with an ENPP1 inhibitor compound, which latter treatment may be administered prior to, concurrently with, or subsequent to adoptive immunotherapy.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug or additional therapy with a pharmaceutical composition of the present disclosure means administration of the compound and second agent or additional therapy at such time that both the known drug and the composition of the present invention will have a therapeutic effect. Such concomitant administration may involve concurrent (i.e. at the same time), prior, or subsequent administration of the drug with respect to the administration of a subject compound. Routes of administration of the two agents may vary, where representative routes of administration are described in greater detail below. A person of ordinary skill in the art would have no difficulty determining the appropriate timing, sequence and dosages of administration for particular drugs or therapies and compounds of the present disclosure.

In some embodiments, the compounds (e.g., a subject compound and the at least one additional compound or therapy) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

Also provided are pharmaceutical preparations of the subject compounds and the second active agent. In pharmaceutical dosage forms, the compounds may be administered in the form of their pharmaceutically acceptable salts, or they may also be used alone or in appropriate association, as well as in combination, with other pharmaceutically active compounds.

In conjunction with any of the subject methods, the ENPP1 inhibitor compounds (e.g., as described herein) (or pharmaceutical compositions comprising such compounds) can be administered in combination with another drug designed to reduce or prevent inflammation, treat or prevent chronic inflammation or fibrosis, or treat cancer. In each case, the ENPP1 inhibitor compound can be administered prior to, at the same time as, or after the administration of the other drug. In certain cases, the cancer is selected from adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioma, glioblastomas, melanoma and various head and neck tumors.

For the treatment, prevention, or reduction of metastasis, the ENPP1 inhibitor compounds can be administered in combination with a chemotherapeutic agent selected from the group consisting of alkylating agents, nitrosoureas, antimetabolites, antitumor antibiotics, plant (vinca) alkaloids, steroid hormones, taxanes, nucleoside analogs, steroids, anthracyclines, thyroid hormone replacement drugs, thymidylate-targeted drugs, Chimeric Antigen Receptor/T cell therapies, Chimeric Antigen Receptor/NK cell therapies, apoptosis regulator inhibitors (e.g., B cell CLL/lymphoma 2 (BCL-2) BCL-2-like 1 (BCL-XL) inhibitors), CARP-1/CCAR1 (Cell division cycle and apoptosis regulator 1) inhibitors, colony-stimulating factor-1 receptor (CSF1R) inhibitors, CD47 inhibitors, cancer vaccine (e.g., a Th17-inducing dendritic cell vaccine, or a genetically modified tyrosinase such as Oncept®) and other cell therapies.

Specific chemotherapeutic agents of interest include, but are not limited to, Gemcitabine, Docetaxel, Bleomycin, Erlotinib, Gefitinib, Lapatinib, Imatinib, Dasatinib, Nilotinib, Bosutinib, Crizotinib, Ceritinib, Trametinib, Bevacizumab, Sunitinib, Sorafenib, Trastuzumab, Ado-trastuzumab emtansine, Rituximab, Ipilimumab, Rapamycin, Temsirolimus, Everolimus, Methotrexate, Danorubicin, Doxorubicin, Epirubicin, Idarubicin, Mitoxantrone, Valrubicin, Abraxane, Folfirinox, Cisplatin, Carboplatin, 5-fluorouracil, Teysumo, Paclitaxel, Prednisone, Levothyroxine, Pemetrexed, navitoclax, and ABT-199. Peptidic compounds can also be used. Cancer chemotherapeutic agents of interest include, but are not limited to, dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD).

In some embodiments, the ENPP1 inhibitor compounds can be administered in combination with a chemotherapeutic agent to treat or reduce metastasis. In some cases, the chemotherapeutic agent is a cytotoxic agent. In certain cases, the chemotherapeutic agent is Gemcitabine. In some cases, the chemotherapeutic agent is Docetaxel. In some cases, the chemotherapeutic agent is Abraxane. In certain cases, the chemotherapeutic agent is doxorubicin. In certain cases, the chemotherapeutic agent is cisplatin.

For the treatment of cancer (e.g., solid tumor cancer or lymphoma), the ENPP1 inhibitor compound can be administered in combination an immunotherapeutic agent. An immunotherapeutic agent is any convenient agent that finds use in the treatment of disease by inducing, enhancing, or suppressing an immune response. In some cases, the immunotherapeutic agent is an immune checkpoint inhibitor. For example, FIG. 2 illustrates that an exemplary ENPP1 inhibitor can act synergistically with an immune checkpoint inhibitor in a mouse model. Any convenient checkpoint inhibitors can be utilized, including but not limited to, cytotoxic T-lymphocyte-associated antigen 4 (CTLA-4) inhibitors, programmed death 1 (PD-1) inhibitors and PD-L1 inhibitors. In certain instances, the checkpoint inhibitor is selected from a cytotoxic T-lymphocyte-associated antigen 4 (CTLA-4) inhibitor, a programmed death 1 (PD-1) inhibitor and a PD-L1 inhibitor. Exemplary checkpoint inhibitors of interest include, but are not limited to, ipilimumab, pembrolizumab and nivolumab. In certain embodiments, for treatment of cancer and/or inflammatory disease, the immunomodulatory polypeptide(s) can be administered in combination with a colony-stimulating factor-1 receptor (CSF1R) inhibitor. CSF1R inhibitors of interest include, but are not limited to, emactuzumab.

Also of interest are agents that are CARP-1/CCAR1 (Cell division cycle and apoptosis regulator 1) inhibitors, including but not limited to those described by Rishi et al., Journal of Biomedical Nanotechnology, Volume 11, Number 9, September 2015, pp. 1608-1627(20), and CD47 inhibitors, including, but not limited to, anti-CD47 antibody agents such as Hu5F9-G4.

In certain instances, the combination provides an enhanced effect relative to either component alone; in some cases, the combination provides a supra-additive or synergistic effect relative to the combined or additive effects of the components. A variety of combinations of the subject compounds and the chemotherapeutic agent may be employed, used either sequentially or simultaneously. For multiple dosages, the two agents may directly alternate, or two or more doses of one agent may be alternated with a single dose of the other agent, for example. Simultaneous administration of both agents may also be alternated or otherwise interspersed with dosages of the individual agents. In some cases, the time between dosages may be for a period from about 1-6 hours, to about 6-12 hours, to about 12-24 hours, to about 1-2 days, to about 1-2 week or longer following the initiation of treatment.

### Combination Radiation Therapy

Alternatively, for the methods of treating cancer, the ENPP1 inhibitor compounds (or pharmaceutical compositions comprising such compounds) can be administered in combination with radiation therapy. In certain embodiments, the methods include administering radiation therapy to the subject. Again, the ENPP1 inhibitor compound can be administered prior to, or after the administration of the radiation therapy. As such, the subject methods can further include administering radiation therapy to the subject. The combination of radiation therapy and administration of the subject compounds can provide a synergistic therapeutic effect. When a subject is exposed to radiation of a suitable dosage and/or frequency during radiation therapy (RT), the production of 2′3′-cGAMP can be induced in the subject. These induced levels of cGAMP can be maintained and/or enhanced when the subject ENPP1 inhibitor compounds are co-administered to prevent the degradation of the cGAMP, e.g., enhanced by comparison to levels achieved with RT alone. For example, FIG. 1 illustrates that an exemplary ENPP1 inhibitor can act synergistically with Radiation therapy (RT) to decrease tumor burden in a mouse model. As such, aspects of the subject methods include administration of a reduced dosage and/or frequency/regimen of radiation treatment as compared to a therapeutically effective dosage and/or frequency/regimen of radiation treatment alone. In some cases, the radiation therapy is administered in combination with the subject compounds at a dosage and/or frequency effective to reduce risk of radiation damage to the subject, e.g., radiation damage that would be expected to occur under a therapeutically effective dosage and/or frequency/regimen of radiation treatment alone.

In some cases, the method includes administering an ENPP1 inhibitor to the subject before radiation therapy. In some cases, the method includes administering an ENPP1 inhibitor to the subject following exposure of the subject to radiation therapy. In certain cases, the method includes sequential administration of radiation therapy, followed by an ENPP1 inhibitor, followed by a checkpoint inhibitor to a subject in need thereof.

### Utility

The compounds and methods of the invention, e.g., as described herein, find use in a variety of applications. Applications of interest include, but are not limited to: research applications and therapeutic applications. Methods of the invention find use in a variety of different applications including any convenient application where inhibition of ENPP1 is desired.

The subject compounds and methods find use in a variety of research applications. The subject compounds and methods may be used in the optimization of the bioavailability and metabolic stability of compounds.

The subject compounds and methods find use in a variety of therapeutic applications. Therapeutic applications of interest include those applications in cancer treatment. As such, the subject compounds find use in the treatment of a variety of different conditions in which the inhibition and/or treatment of cancer in the host is desired. For example, the subject compounds and methods may find use in treating a solid tumor cancer (e.g., as described herein), such as a lymphoma.

### Pharmaceutical Compositions

The herein-discussed compounds can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In some embodiments, the subject compound and a second active agent (e.g., as described herein), e.g. a small molecule, a chemotherapeutic, an antibody, an antibody fragment, an antibody-drug conjugate, an aptamer, or a protein, etc. are administered to individuals in a formulation (e.g., in the same or in separate formulations) with a pharmaceutically acceptable excipient(s). In some embodiments, the second active agent is a checkpoint inhibitor, e.g., a cytotoxic T-lymphocyte-associated antigen 4 (CTLA-4) inhibitor, a programmed death 1 (PD-1) inhibitor, or a PD-L1 inhibitor.

In certain cases, two or more therapeutic agents (e.g., cGAS ligands, STING ligands, and/or ENPP1 inhibitors) can be co-formulated. In certain cases, all of the therapeutic agents (e.g., cGAS ligands, STING ligands, and/or ENPP1 inhibitors) are co-formulated. In certain cases, two or more therapeutic agents can be administered as separate formulations.

In another aspect of the present invention, a pharmaceutical composition is provided, comprising, or consisting essentially of, a compound of the present invention, or a pharmaceutically acceptable salt, isomer, tautomer or prodrug thereof, and further comprising one or more additional active agents of interest. Any convenient active agents can be utilized in the subject methods in conjunction with the subject compounds. In some instances, the additional agent is a checkpoint inhibitor. The subject compound and checkpoint inhibitor, as well as additional therapeutic agents as described herein for combination therapies, can be administered orally, subcutaneously, intramuscularly, intranasally, parenterally, or other route. The subject compound and second active agent (if present) may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ. In some cases, the therapeutic agents can be administered intratumorally. In some embodiments, the therapeutic agents are administered parenterally. In some embodiments, the therapeutic agents are administered subcutaneously.

In certain cases, the therapeutic agents can be administered as a pharmaceutical composition formulated for mucosal delivery. Examples of mucosal delivery of cGAS/STING pathway agonists are described in more detail in Martin et al. (Vaccine. 2017 Apr. 25; 35(18): 2511-2519) and Dubensky et al. (Ther Adv Vaccines (2013) 1(4) 131-143), each herein incorporated by reference for all purposes. Mucosal delivery can include, but is not limited to, buccal delivery, sublingual delivery, or intranasal delivery. In certain cases, the therapeutic agents can be administered buccally. In certain cases, the therapeutic agents can be administered sublingually. In certain cases, the therapeutic agents can be administered intranasally. Pharmaceutical compositions formulated for mucosal delivery can include formulation in a nanoparticle, such as liposomes. Liposomes useful for mucosal delivery are known to those skilled in the art. For example, liposomes useful for mucosal delivery can contain a pulmonary surfactant, a pulmonary surfactant membrane constituent, and/or a pulmonary surfactant biomimetic are described in more detail in Wang et al. [Science 367, 869 (2020)], herein incorporated by reference for all purposes. In certain cases, two or more therapeutic agents (e.g., cGAS ligands, STING ligands, and/or ENPP1 inhibitors) can be co-formulated for mucosal delivery. In certain cases, all of the therapeutic agents (e.g., cGAS ligands, STING ligands, and/or ENPP1 inhibitors) are co-formulated for mucosal delivery. In certain cases, two or more therapeutic agents can be administered as separate formulations for mucosal delivery.

In some embodiments, the subject compound and a chemotherapeutic agent are administered to individuals in a formulation (e.g., in the same or in separate formulations) with a pharmaceutically acceptable excipient(s). The chemotherapeutic agents include, but are not limited to alkylating agents, nitrosoureas, antimetabolites, antitumor antibiotics, plant (vinca) alkaloids, and steroid hormones. Peptidic compounds can also be used. Suitable cancer chemotherapeutic agents include dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD).

The subject compound and second chemotherapeutic agent, as well as additional therapeutic agents as described herein for combination therapies, can be administered orally, subcutaneously, intramuscularly, parenterally, or other route. The subject compound and second chemotherapeutic agent may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

### Definitions

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of tumor burden. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction in of tumor burden).

As used herein, the term “metastasis” refers to the propagation of a cancer from the organ where it started to a different organ. It generally occurs through the blood or lymphatic system. When the cancer cells spread and form a new tumor, the latter is called a secondary or metastatic tumor. The cancer cells forming the secondary tumor are like those of the original tumor. If a breast cancer, for example, spreads (metastasizes) to the lung, the secondary tumor is formed of malignant breast cancer cells. The disease in the lung is metastatic breast cancer and not lung cancer. The activity to generate a metastatic lesion and promote metastasis includes release of cancer cells from the primary site, intravasation to neighboring vessels, transport to the site of metastasis through blood flow, and extravasation and/or infarction to the distant organ. Analysis of the subject can detect occurrence or promotion of metastasis, growth or proliferation of the cells, increase in colony forming activity, etc.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intratracheal, intramuscular, subcutaneous, and the like.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

The term “alkyl” refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “substituted alkyl” is meant to include an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

The term “alkenyl” refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” refers to an alkyl group bound through a single, terminal ether linkage; that is, an “alkoxy” group may be represented as —O-alkyl where alkyl is as defined above. A “lower alkoxy” group refers to an alkoxy group containing 1 to 6 carbon atoms, and includes, for example, methoxy, ethoxy, n-propoxy, isopropoxy, t-butyloxy, etc. Substituents identified as “C1-C6 alkoxy” or “lower alkoxy” herein may, for example, may contain 1 to 3 carbon atoms, and as a further example, such substituents may contain 1 or 2 carbon atoms (i.e., methoxy and ethoxy).

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “aryl”, unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl; which may further be substituted with one to five members selected from the group consisting of hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

The term “aralkyl” refers to an alkyl group with an aryl substituent, and the term “alkaryl” refers to an aryl group with an alkyl substituent, wherein “alkyl” and “aryl” are as defined above. In general, aralkyl and alkaryl groups herein contain 6 to 30 carbon atoms. Aralkyl and alkaryl groups may, for example, contain 6 to 20 carbon atoms, and as a further example, such groups may contain 6 to 12 carbon atoms.

The term “alkylene” refers to a di-radical alkyl group. Unless otherwise indicated, such groups include saturated hydrocarbon chains containing from 1 to 24 carbon atoms, which may be substituted or unsubstituted, may contain one or more alicyclic groups, and may be heteroatom-containing. “Lower alkylene” refers to alkylene linkages containing from 1 to 6 carbon atoms. Examples include, methylene (—CH₂—), ethylene (—CH₂CH₂—), propylene (—CH₂CH₂CH₂—), 2-methylpropylene (—CH₂—CH(CH₃)—CH₂—), hexylene (—(CH₂)₆—) and the like.

Similarly, the terms “alkenylene,” “alkynylene,” “arylene,” “aralkylene,” and “alkarylene” refer to di-radical alkenyl, alkynyl, aryl, aralkyl, and alkaryl groups, respectively.

The term “amino” refers to the group —NRR′ wherein R and R′ are independently hydrogen or nonhydrogen substituents, with nonhydrogen substituents including, for example, alkyl, aryl, alkenyl, aralkyl, and substituted and/or heteroatom-containing variants thereof.

The terms “halo” and “halogen” are used in the conventional sense to refer to a chloro, bromo, fluoro or iodo substituent.

“Carboxyl,” “carboxy” or “carboxylate” refers to —CO₂H or salts thereof.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocycloalkyl” refers to a cycloalkyl substituent that is heteroatom-containing, the terms “heterocyclic” or “heterocycle” refer to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The terms “heterocycle,” “heterocyclic” and “heterocyclyl” refer to a saturated or unsaturated group having a single ring or multiple condensed rings, including fused bridged and spiro ring systems, and having from 3 to 15 ring atoms, including 1 to 4 hetero atoms. These ring heteroatoms are selected from nitrogen, sulfur and oxygen, wherein, in fused ring systems, one or more of the rings can be cycloalkyl, heterocycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. A hydrocarbyl may be substituted with one or more substituent groups. The term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cycloalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH₂), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N+≡C—), cyanato (—O≡C—N), isocyanato (—O—N+≡C—), isothiocyanato (—S≡C—N), azido (—N═N+═N—), formyl (—(CO)—H), thioformyl (—(CS)—H), amino (—NH₂), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO₂-alkyl), C5-C20 arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

By “linking” or “linker” as in “linking group,” “linker moiety,” etc., is meant a linking moiety that connects two groups via covalent bonds. The linker may be linear, branched, cyclic or a single atom. Examples of such linking groups include alkyl, alkenylene, alkynylene, arylene, alkarylene, aralkylene, and linking moieties containing functional groups including, without limitation: amido (—NH—CO—), ureylene (—NH—CO—NH—), imide (—CO—NH—CO—), epoxy (—O—), epithio (—S—), epidioxy (—O—O—), carbonyldioxy (—O—CO—O—), alkyldioxy (—O—(CH₂)n-O—), epoxyimino (—O—NH—), epimino (—NH—), carbonyl (—CO—), etc. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, poly(ethylene glycol) unit(s) (e.g., —(CH₂—CH₂—O)—); ethers, thioethers, amines, alkyls (e.g., (C₁-C₁₂)alkyl), which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable. Any convenient orientation and/or connections of the linkers to the linked groups may be used.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻ M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻ M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻ M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰,

trihalomethyl, —CF₃, —CN, —OCN, —SCN, —NO, —NO₂, —N₃, —SO₂R⁷⁰, —SO₃⁻
M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻
M⁺, —P(O)(OR⁷⁰)₂, —C(O)R⁷⁰, —C(S)R⁷⁰, —C(NR⁷⁰)R⁷⁰, —CO₂⁻
M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻
M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻
M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰,

trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰) R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —N R⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

As used herein, the term “anti-tumor effect” or “anti-tumor activity” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor ceil survival, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place.

As used herein, the term “affinity” refers to a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, including use of binding experiments to calculate affinity. Antibody activity in functional assays (e.g., flow cytometry assay) is also reflective of antibody affinity. Antibodies and affinities can be phenotypically characterized and compared using functional assays (e.g., flow cytometry assay).

As used herein, the term “immunosuppressive activity” refers to the induction of signal transduction or changes in protein expression in a cell, such as an activated immune effector cell, that results in a decrease in an immune response. Non-limiting examples of polypeptides known to suppress or decrease an immune response via their binding include CD47, PD-1, CTLA-4, and their corresponding ligands, including SIRPa, PD-L1, PD-L2, B7-1, and B7-2. Such polypeptides may be present in the tumor microenvironment and can inhibit immune responses to neoplastic cells. In various embodiments, inhibiting, blocking, or otherwise antagonizing the interaction of immunosuppressive polypeptides and/or their ligands may enhance the immune response of the immune effector cell.

As used herein, the term “enzymatic inhibitory domain” refers to a protein domain that inhibits an intracellular signal transduction cascade, for example a native T cell activation cascade. In some embodiments, the enzymatic inhibitory domain of a chimeric inhibitory receptor of the present disclosure comprises at least a portion of an extracellular domain, a transmembrane domain, and/or an intracellular domain. In some embodiments, the enzymatic inhibitory domain comprises at least a portion of an enzyme. In some embodiments, the enzyme is selected from CSK, SHP-1, PTEN, CD45, CD148, PTP-MEG1, PTP-PEST, c-CBL, CBL-b, PTPN22, LAR, PTPH1, SHIP-1, and RasGAP (see e.g., Stanford et al., Regulation of TCR signaling by tyrosine phosphatases: from immune homeostasis to autoimmunity, Immunology, 2012 September; 137(1): 1-19). In some embodiments, the portion of the enzyme comprises an enzyme domain(s), an enzyme fragment(s), or a mutant(s) thereof. In some embodiments, the portion of the enzyme is a catalytic domain of the enzyme. In some embodiments, the enzyme domain(s), enzyme fragment(s), or mutants(s) thereof are selected to maximize efficacy and minimize basal inhibition.

As used herein, the term “immunostimulatory activity” refers to induction of signal transduction or changes in protein expression in a cell, such as an activated immune effector cell, that results in an increase in an immune response. Immunostimulatory activity may include pro-inflammatory activity. Non-limiting examples of polypeptides known to stimulate or increase an immune response via their binding include CD28, OX-40, 4-1BB, and their corresponding ligands, including B7-1, B7-2, OX-40L, and 4-1BBL. Such polypeptides may be present in the tumor microenvironment and can activate immune responses to neoplastic cells. In various embodiments, promoting, stimulating, or otherwise agonizing pro-inflammatory polypeptides and/or their ligands may enhance the immune response of the immune effector cell.

Isolated nucleic acid molecules of the present disclosure include any nucleic acid molecule that encodes a polypeptide of the present disclosure, or fragment thereof. Such nucleic acid molecules need not be 100% homologous or identical with an endogenous nucleic acid sequence, but will typically exhibit substantial identity. Nucleic acids having “substantial identity” or “substantial homology” to an endogenous sequence are typically capable of hybridizing with at least one strand of a double-stranded nucleic acid molecule. As used herein, “hybridize” refers to pairing to form a double-stranded molecule between complementary polynucleotide sequences (e.g., a gene described herein), or portions thereof, under various conditions of stringency. For example, stringent salt concentration may be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide or at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency may be accomplished by combining these various conditions as needed.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). Preferably, such a sequence is at least about 60%, about 80%, about 85%, about 90%, about 95%, about 99%, or about 100% homologous or identical at the amino acid level or nucleic acid to the sequence used for comparison. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of rnRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the term “ligand” refers to a molecule that binds to a receptor. In particular, the ligand binds a receptor on another cell, allowing for cell-to-cell recognition and/or interaction.

Definitions of other terms and concepts appear throughout the detailed description.

All references, issued patents and patent applications cited within the body of the specification are hereby incorporated by reference in their entirety, for all purposes.

### EXAMPLES

The following examples are put forth so as to provide those of ordinary skill in the art with a complete disclosure and description of how to make and use embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present disclosure. All such modifications are intended to be within the scope of the claims appended hereto.

### Example 1: Synthesis of Compound 1

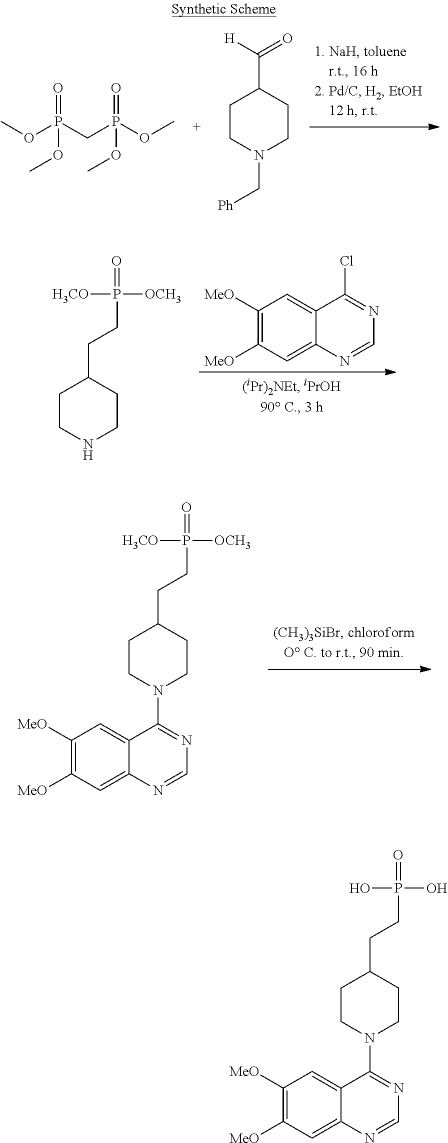

### Preparation of dimethyl (2-(piperidin-4-yl)ethyl)phosphonate

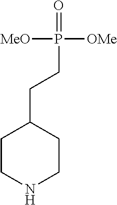 [SMILES: COP(=O)(CCC1CCNCC1)OC]

Sodium hydride (2.16 g, 54.11 mmol) was carefully added to a stirred solution of bis(dimethoxyphosphoryl)methane (11.42 g, 49.19 mmol) in toluene (100 mL) at room temperature. The reaction mixture was then placed under an atmosphere of nitrogen and a solution of 1-benzylpiperidine-4-carbaldehyde (10 g, 49.19 mmol) in toluene (50 mL) was slowly added keeping the temperature below 40° C. The resulting mixture was left to stir at room temperature for 16 h and then quenched by the addition of aqueous saturated ammonium chloride solution. The organic phase was separated, washed with brine, dried (MgSO₄) and evaporated to dryness. Chromatography (120 g SiO₂; 5 to 100% gradient of EtOAc in hexanes) provided dimethyl (E)-(2-(1-benzylpiperidin-4-yl)vinyl)phosphonate (6.2 g, 16%) as a colorless oil.

To a mixture of dimethyl (E)-(2-(1-benzylpiperidin-4-yl)vinyl)phosphonate (3.7 g, 12.0 mmol) in ethanol (40 mL) was added Pd/C (1.1 g, 10.3 mmol). The mixture was placed under an atmosphere of hydrogen and stirred at room temperature for 12 h, filtered and evaporated to dryness under reduced pressure to give dimethyl (2-(piperidin-4 yl)ethyl)phosphonate (2.7 g, 100%) as colorless oil.

### Preparation of dimethyl (2-(1-(6,7-dimethoxyquinazolin-4-yl)piperidin-4-yl)ethyl)phosphonate

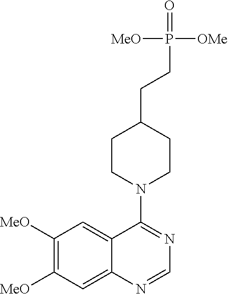 [SMILES: COc1cc2ncnc(N3CCC(CCP(=O)(OC)OC)CC3)c2cc1OC]

Diisopropylethylamine (0.6 g, 8.9 mmol) was added to a mixture of dimethyl (2-(piperidin-4-yl)ethyl)phosphonate (1.1 g, 4.9 mmol) and 4-chloro-6,7-dimethoxyquinazoline (1.0 g, 4.5 mmol) in isopropyl alcohol (20 mL). After stirring at 90° C. for 3 h, the reaction mixture was cooled and evaporated to dryness. Purification of silica gel (5% MeOH in dichloromethane) provided dimethyl (2-(1-(6,7-dimethoxyquinazolin-4-yl)piperidin-4-yl)ethyl)phosphonate (755 mg, 37%) as oil. LC-MS: m/z=410.25 [M+H]⁺

¹H NMR (500 MHz, CDCl₃) δ 8.65 (s, 1H), 7.23 (s, 1H), 7.09 (s, 1H), 4.19 (dq, J=14.0, 2.9, 2.4 Hz, 2H), 4.02 (s, 3H), 3.99 (s, 3H), 3.77 (s, 3H), 3.75 (s, 3H), 3.05 (td, J=12.8, 2.3 Hz, 2H), 1.93-1.77 (m, 4H), 1.67 (ddd, J=14.1, 9.5, 5.9 Hz, 3H), 1.46 (qd, J=12.2, 3.7 Hz, 2H).

### Preparation of dimethyl (2-(1-(6,7-dimethoxyquinazolin-4-yl)piperidin-4-yl)ethyl)phosphonic Acid (Compound 1)

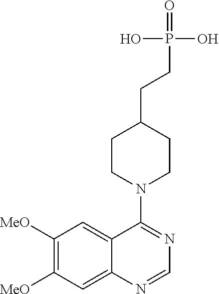 [SMILES: COc1cc2ncnc(N3CCC(CCP(=O)(O)O)CC3)c2cc1OC]

Bromotrimethylsilane (3.67 g, 24 mmol) was added to a cooled solution of dimethyl (2-(1-(6,7-dimethoxyquinazolin-4-yl)piperidin-4-yl)ethyl)phosphonate (3.25 g, 7.94 mmol) in chloroform (60 mL) that was cooled by an ice bath. The reaction mixture was allowed to warm to room temperature and after 90 minutes was quenched by the addition of methanol (20 mL). The mixture was evaporated to dryness under reduced pressure and then solvated in methanol (100 mL). The reaction mixture was concentrated to half volume, filtered to remove precipitate, and then evaporated to dryness. The residue was crystalized with dichloromethane, filtered and dried under vacuum to give dimethyl (2-(1-(6,7-dimethoxyquinazolin-4-yl)piperidin-4-yl)ethyl)phosphonic acid (2.1 g, 69%).

LC-MS: m/z=381.8 [M+H]⁺

¹H NMR (500 MHz, DMSO-d₆) δ 8.77 (s, 1H), 7.34 (s, 1H), 7.23 (s, 1H), 4.71 (d, J=13.1 Hz, 2H), 3.99 (s, 3H), 3.97 (s, 3H), 3.48 (t, J=12.7 Hz, 2H), 3.18 (s, 1H), 1.97-1.90 (m, 2H), 1.62-1.43 (m, 4H), 1.40-1.27 (m, 2H).

### Example 2: Assessing Compound Activity

Selected compounds of Table 1, Table 2 and other derivatives were prepared and assessed in an ENPP1 activity assay using thymidine monophosphate paranitrophenol (TMP-pNP) as a substrate. Enzyme reactions were prepared with TMP-pNP (2 μM), 5-fold dilutions of ENPP1 inhibitor, and purified recombinant mouse ENPP1 (0.5 nM) in 100 mM Tris, 150 mM NaCl, 2 mM CaCl₂), 200 μM ZnCl₂, pH 7.5 at room temperature. Reaction progress was monitored by measuring absorbance at 400 nm of paranitrophenolate produced by the reaction for 20 minutes. Slopes of product formation were extracted, plotted, and fit to obtain IC₅₀ values with Graphpad Prism 7.03.

Compounds were also assessed in an ENPP1 enzyme activity assay using ³²P cGAMP as a substrate. Radiolabeled ³²P cGAMP was synthesized by incubating unlabeled ATP (1 mM) and GTP (1 mM) doped with ³²P-ATP with 2 μM purified recombinant porcine cGAS in 20 mM Tris pH 7.5, 2 mM MgCl₂, 100 μg/mL herring testes DNA) overnight at room temperature, and the remaining nucleotide starting materials were degraded with alkaline phosphatase for 4 h at 37° C. The probe ³²P-cGAMP (5 μM) was incubated with purified recombinant mouse ENPP1 (20 nM) or whole cell lysates in 100 mM Tris, 150 mM NaCl, 2 mM CaCl₂), 200 μM ZnCl₂, pH 7.5 at room temperature for 5 hours. To generate enzyme inhibition curves, 5-fold dilutions of ENPP1 inhibitor were included in the reaction. Degradation was evaluated by TLC (as described by L1 et al. Nat. Chem. Biol. (2014) 10:1043-8). Plates were exposed on a phosphor screen (Molecular Dynamics) and imaged on a Typhoon 9400 and the ³²P signal was quantified using ImageJ. Inhibition curves were fit to obtain IC₅₀ values using Graphpad Prism 7.03. The IC₅₀ of the compounds tested is provided in table 3. IC₅₀ values fall in the range indicated by letters A-D, where A represents an IC₅₀ value less than 0.005 μM, B represents an IC₅₀ value between 0.005 μM and 0.05 μM, and C represents an IC₅₀ value between 0.05 μM and 0.5 μM, D represents an IC₅₀ value between 0.5 μM and 5 μM, and E represents an IC₅₀ value greater than 5 μM (n.d.=not determined).

TABLE 3
A (<5 nM); B (5 nM-50 nM); C (50 nM-500 nM);
D (500 nM-5 μM); E (>5 μM)

| | IC₅₀ values | IC₅₀ values |
|---|---|---|
| Compound | (TMP-pNP; μM) | (cGAMP; μM) |
| 1 | A | C |
| 5 | C | n.d. |
| 7 | C | n.d. |
| 10 | A | C |
| 11 | E | n.d. |
| 12 | B | C |
| 13 | D | n.d. |
| 16 | D | n.d. |
| 18 | A | C |
| 19 | B | n.d. |
| 22 | C | n.d. |
| 23 | D | n.d. |
| 25 | B | n.d. |
| 30 | C | n.d. |
| 38 | C | n.d. |
| 42 | E | n.d. |
| 60 | C | n.d. |
| 61 | D | n.d. |
| 67 | B | n.d. |
| 68 | E | n.d. |
| 70 | D | n.d. |
| 71 | C | n.d. |
| 72 | n.d. | n.d. |
| 73 | E | n.d. |
| 74 | n.d. | n.d. |
| 75 | C | D |
| 76 | A | B |
| 77 | n.d. | n.d. |
| 78 | n.d. | n.d. |
| 81 | B | n.d. |
| 82 | B | n.d. |
| 83 | C | n.d. |
| 84 | C | n.d. |
| 86 | A | n.d. |
| 87 | C | n.d. |
| 88 | C | n.d. |
| 92 | A | n.d. |
| 93 | C | n.d. |
| 103 | C | n.d. |

### Example 3: Testing of Compound 76 (Cpd 76) in EMT-6 Tumor Model

Test animals were female Balb/c mice aged 5-8 weeks at inoculation.

### Cell Culture

The EMT6 tumor cells were maintained in vitro with DMEM medium supplemented with 10% fetal bovine serum at 37° C. in an atmosphere of 5% CO₂ in air. The cells in exponential growth phase were harvested and quantitated by cell counter before tumor inoculation.

### Tumor Inoculation

Each mouse was inoculated subcutaneously in the right flank region with EMT6 tumor cells (5×10⁵) in 0.1 ml of PBS for tumor development.

Staggered inoculation was performed. G1, 2, 3, 6, 7 were inoculated one day before G4, 5, 8, 9, 10.

### Randomization

The randomization started when the mean tumor size reached approximately 80 (60-110) mm³. 100 mice were enrolled in the study. All animals were randomly allocated to 10 study groups. Randomization was performed based on “Matched distribution” method (StudyDirector™ software, version 3.1.399.19).

Staggered randomization was performed. G1, 2, 3, 6, 7 were randomized one day before G4, 5, 8, 9, 10.

The date of tumor randomization was denoted as day 0.

### Observation and Data Collection

After tumor cells inoculation, the animals were checked daily for morbidity and mortality. During routine monitoring, the animals were checked for any effects of tumor growth and treatments on behavior such as mobility, food and water consumption, body weight gain/loss (body weights were measured twice per week after randomization), eye/hair matting and any other abnormalities. Mortality and observed clinical signs were recorded for individual animals in detail.

Tumor volumes were measured twice per week in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L). (Tumor weight was measured at the end of study). Dosing as well as tumor and body weight measurements were conducted in a Laminar Flow Cabinet.

The study was terminated at 4 weeks post randomization. Individual animals were euthanized when TV>3000 mm³.

### Statistical Analysis

For comparison between two groups, a Student's t-test was performed. For comparison among three or more groups, an one-way ANOVA was performed followed by multiple comparison procedures. All data were analyzed using SPSS 18.0 and/or GraphPad Prism 5.0. P<0.05 is considered statistically significant.

Animals were treated with vehicle, cpd 76, radiotherapy, or anti-PD-L1, or a combination of treatments. Compound 76 was dosed 50 mg/kg and formulated 5 mg/ml in PBS. Radiotherapy used the X-RAD 225Cx SmART Small Animal Radiation Therapy System (Crownbio). Ten animals were in each group and dosing was performed as in Table 4.

TABLE 4

| | | Dose | Dose | | Dose |
|---|---|---|---|---|---|
| | | level | solution | Dose | frequency & |
| Group | Treatment/ROA | (mg/kg | (mg/ml) | volume | duration |
| 1 | Vehicle (PBS)/ | — | — | 10 ul/g | QD, Days 1-7 |
| | s.c. | | | | |
| 2 | Vehicle (PBS)/ | — | | 10 ul/g | QD, Days 1-7 |
| | s.c. | | | | |
| | Radiotherapy/— | 15 Gy | — | — | Single IR dose |
| | | | | | (Day 2) |
| 3 | Cpd 76/s.c. | 50 | 5 | 10 ul/g | QD, 3 days on |
| | | | | | 4 days off for |
| | | | | | 4 weeks |
| 4 | Cpd 76/s.c. | 50 | 5 | 10 ul/g | QD, Days 1-7 |
| 5 | Anti-PD-L1/ip | 2.5 | 0.25 | 10 ul/g | BIW*4 weeks |
| 6 | Radiotherapy/— | 15 Gy | — | — | Single IR dose |
| | | | | | (Day 2) |
| | Cpd 76/i.p. | 50 | 5 | 10 ul/g | QD, 3 days on |
| | | | | | 4 days off for |
| | | | | | 4 weeks |
| 7 | Radiotherapy/— | 15 Gy | — | — | Single IR dose |
| | | | | | (Day 2) |
| | Cpd 76/s.c. | 50 | 5 | 10 ul/g | QD, Day 1-7 |
| 8 | Radiotherapy/— | 15 Gy | — | — | Single IR dose |
| | | | | | (Day 2) |
| | Anti-PD-L1/i.p. | 2.5 | 0.25 | 10 ul/g | BIW*4 weeks |
| 9 | Cpd 76/s.c. | 50 | 5 | 10 ul/g | QD, Days 1-7 |
| | Anti-PD-L1/i.p. | 2.5 | 0.25 | 10 ul/g | BIW*4 weeks |
| 10 | Radiotherapy/— | 15 Gy | — | — | Single IR dose |
| | | | | | (Day 2) |
| | Cpd 76/s.c. | 50 | 5 | 10 ul/g | QD, Days 1-7 |
| | Anti-PD-L1/i.p. | 2.5 | 0.25 | 10 ul/g | BIW*4 weeks |

Notes:
radiation administered one day after Cpd 76 treatment
treatment initiated one day post grouping
Groups 4, 5, 8, 9, 10 inoculated and randomized one day after groups 1, 2, 3, 6, 7.

### Results

Compound 76 (50 mg/kg) monotherapy, given on QD 1-7 only or 3 d on/4 d off for 4 weeks had no effect on tumor volume (FIGS. 1 and 2).

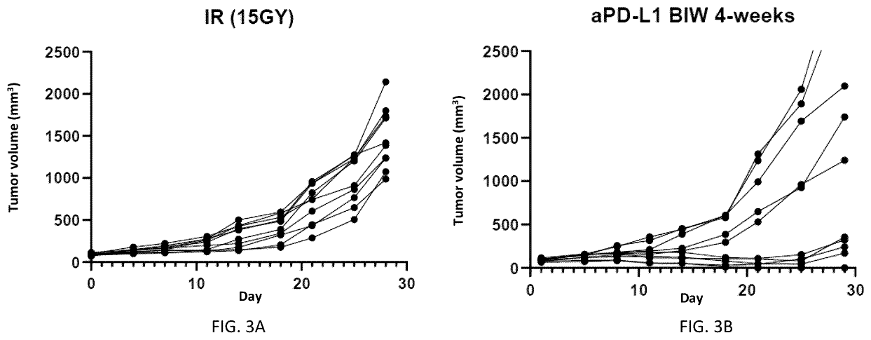

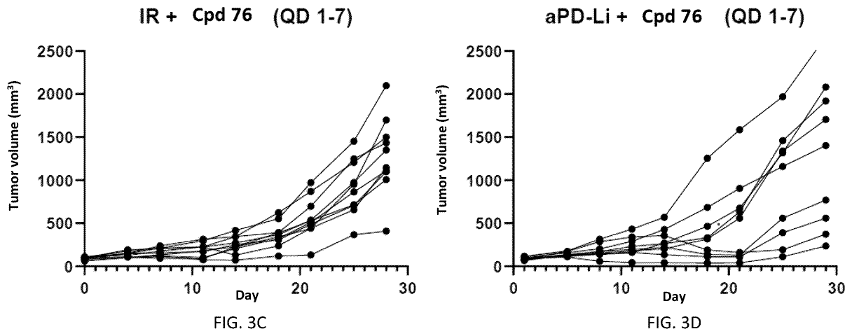

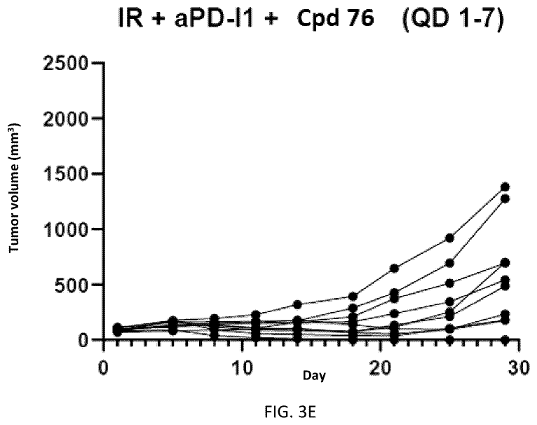

Both irradiation with ionizing radiation (IR) (15 Gy) and aPD-L1 (2.5 mg/kg) significantly inhibited tumor growth independently (FIGS. 1, 2, 3A, 3B), but IR and aPD-L1 combined was no better than aPD-L1 alone (FIGS. 1, 2).

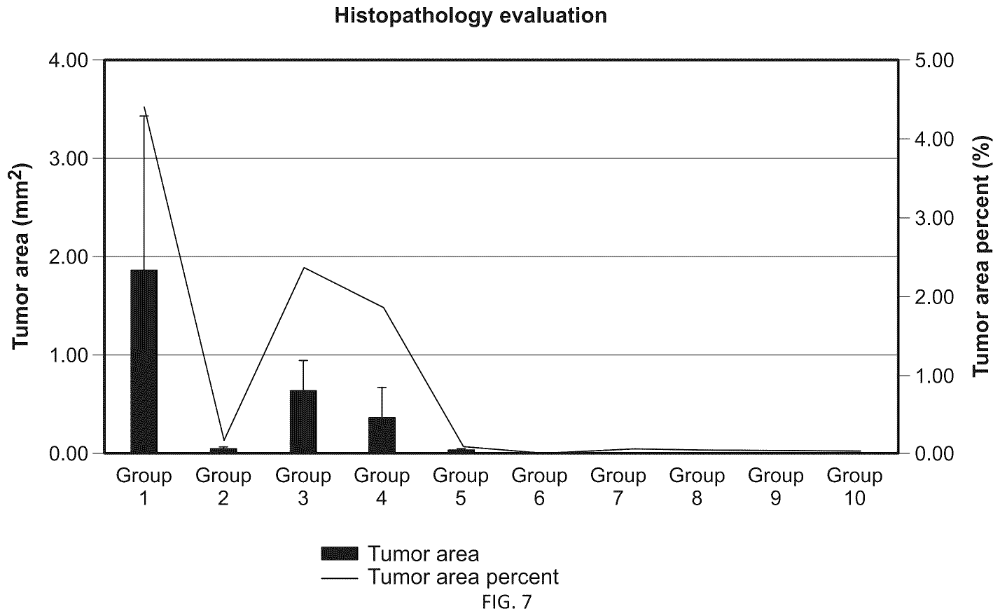

However, Compound 76 (50 mg/kg, QD-1-7) had a synergistic effect when combined with IR and aPD-L1, (“triple combo”) further reducing tumor growth (FIGS. 1, 2, 3A-3E, 7)

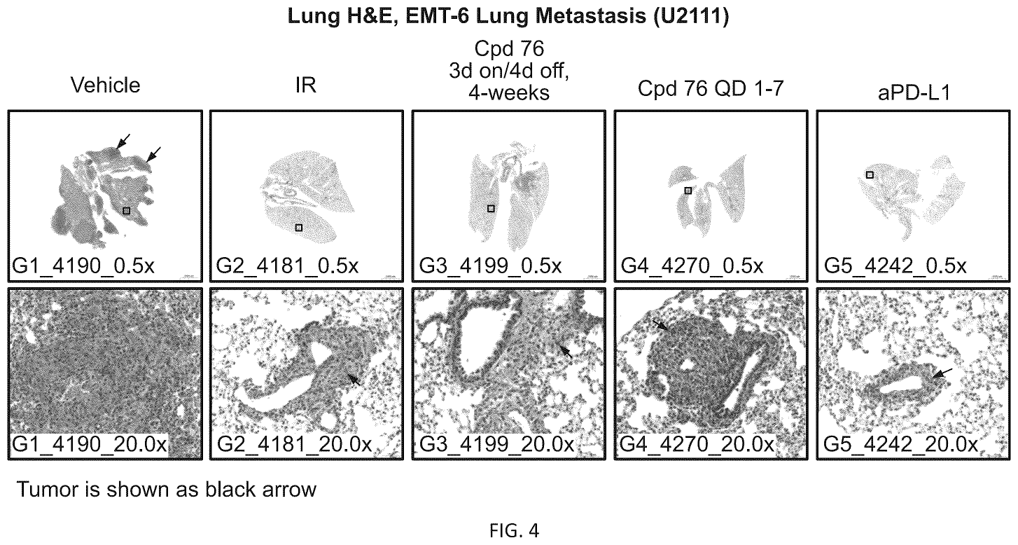

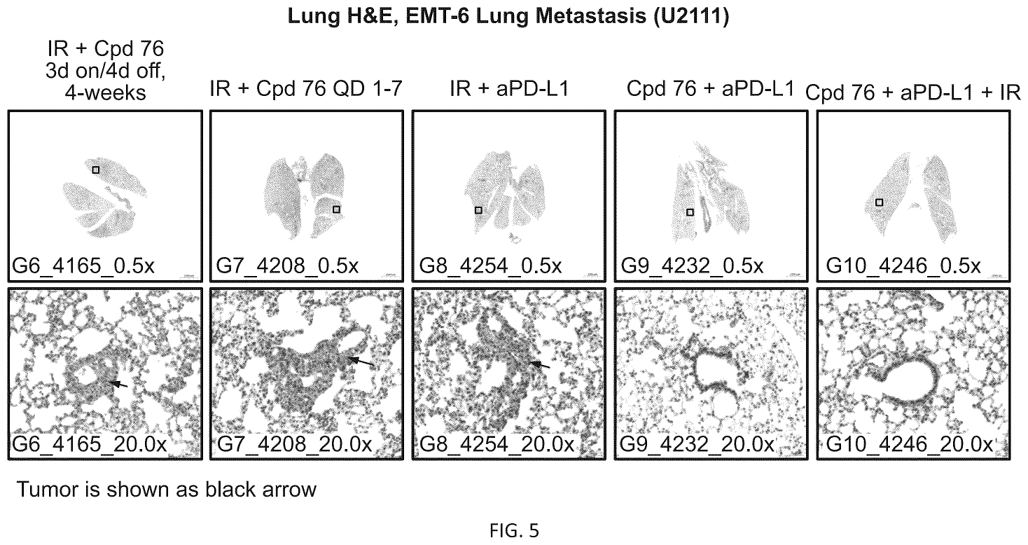

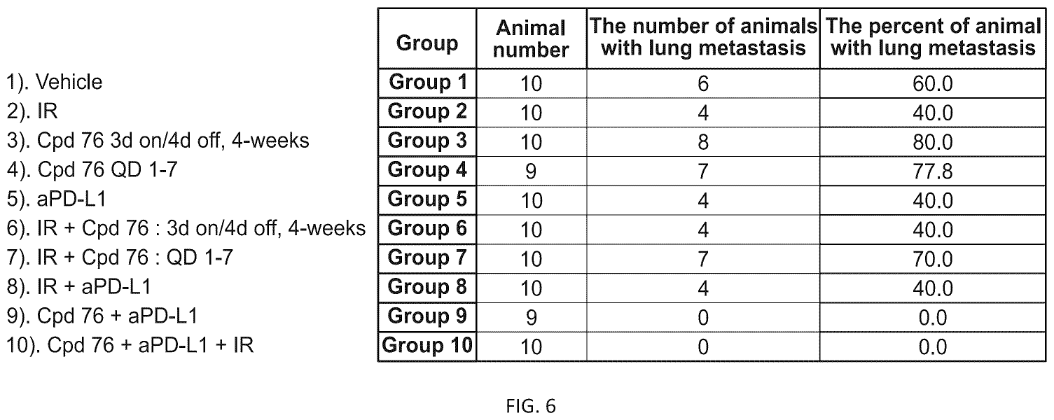

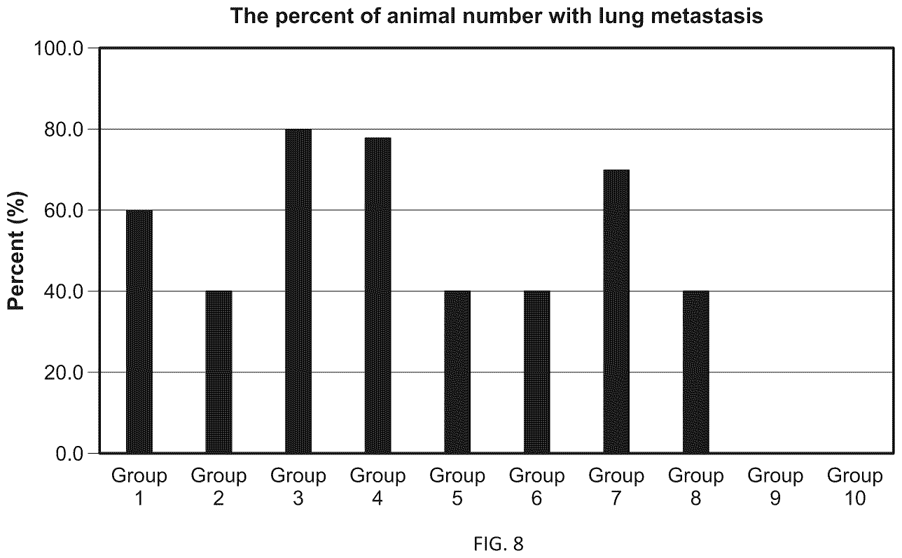

Treatment with aPD-L1 (4/10) and IR (4/10) independently reduced lung metastasis compared to vehicle (6/10) or Compound 76 monotherapy (7/10 and 8/10) (FIGS. 4, 5, 6, 8).

Compound 76 (QD 1-7 d) when combined with aPD-L1 (Groups 9 and 10) further reduced lung metastasis to 0/20 animals compared to aPD-L1 alone (Group 5) which recorded 4/10 at study termination (day 29) (FIGS. 4, 5, 6, 8).

The above data suggest that the use of Compound 76 (QD 1-7 d) combined with a checkpoint inhibitor (CPI) reduces metastasis, and that the use of Compound 76 (QD 1-7 d) combined with a CPI and IR reduces metastasis.

TABLE 5
Lung metastases in EMT-6 Model

| | | | | | Compound |
|---|---|---|---|---|---|
| | | | | Compound | 76 + IR + |
| | Vehicle | IR | aPD-L1 | 76 + aPD-L1 | aPD-L1 |
| % of animals | 60% | 40% | 40% | 0% | 0% |
| with lung | | | | | |
| metastases | | | | | |
| metastatic | 4.41% | 0.15% | 0.09% | 0% | 0% |
| tumor burden | | | | | |
| (% lung) | | | | | |

### Example 4—Testing of AG-3132 in 4T1 Tumor Model

The efficacy of AG-3132, alone and in combination with doxorubicin, against primary tumor growth and tumor metastasis was evaluated in an orthotopic murine breast cancer model with a luciferase reporter gene (4T1-Me_Luc). The purpose of the luciferase reporter gene was to enable quantification of extent of metastasis upon harvest of target organs at study endpoint.

The study consisted of four experimental groups, each containing 12 female BALB/c mice after randomization. 0.5×10⁶ 4T1-M3_Luc tumor cells in 100 μl PBS were implanted into the left mammary fat pad of all participating mice. Tumor growth was monitored by caliper measurement throughout the study.

After animals had been randomized on Day 7, treatments were initiated on the same day. Test compound AG-3132 was administered at a dose of 50 mg/kg subcutaneously once a day on Days 7, 8, 9, 14, 15 and 16 alone (Group 2) and in combination with Doxorubicin at 5 mg/kg intraperitoneally once a day on Days 7 and 14 (Group 4). Doxorubicin was also evaluated alone at 5 mg/kg intraperitoneally once a day on Days 7 and 14 (Group 3). All treatment groups were compared to the vehicle control given subcutaneously once a day on Days 7, 8, 9, 14, 15 and 16 (Group 1).

During the course of the study, individual animals were euthanized due to ethical abortion criteria prior to study end without performing a necropsy. After three animals of Group 3 and one animal of Group 4 had to be euthanized due to excessive weight loss, both Groups were terminated on Day 21 and a necropsy was performed on the terminated animals. On Day 22, one animal of Group 1 was euthanized due ethical abortion criteria (tumor ulceration). After four more animals of Group 1 reached ethical abortion criteria (tumor size) on Day 25, the study was terminated, all remaining animals were sacrificed, and a necropsy was performed.

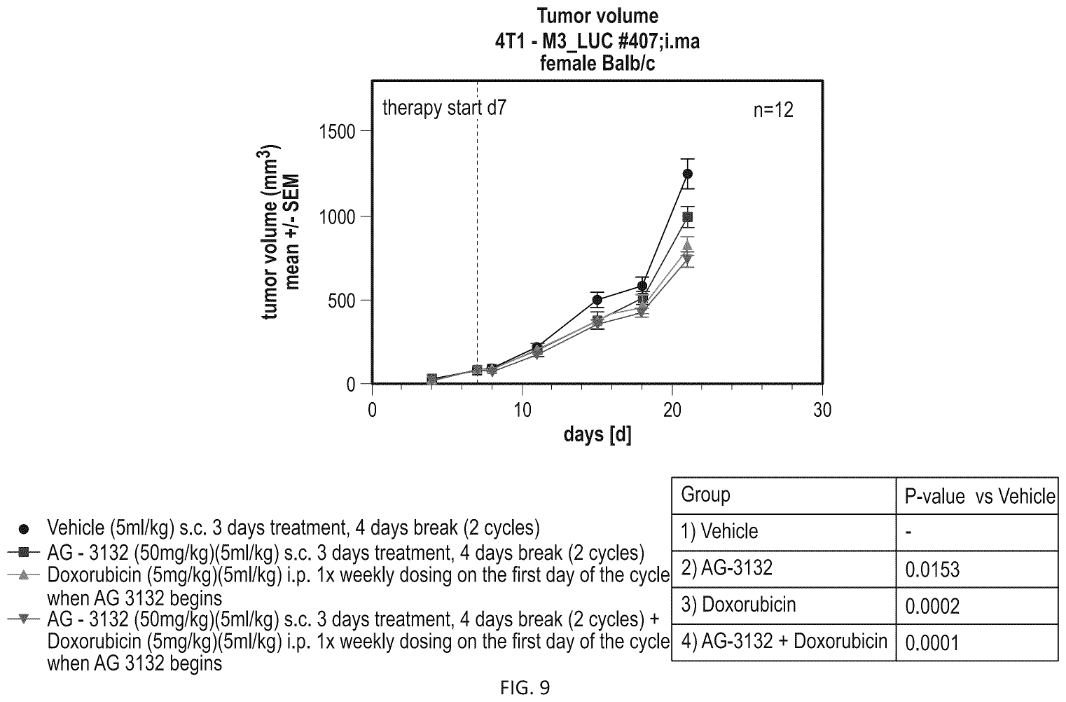

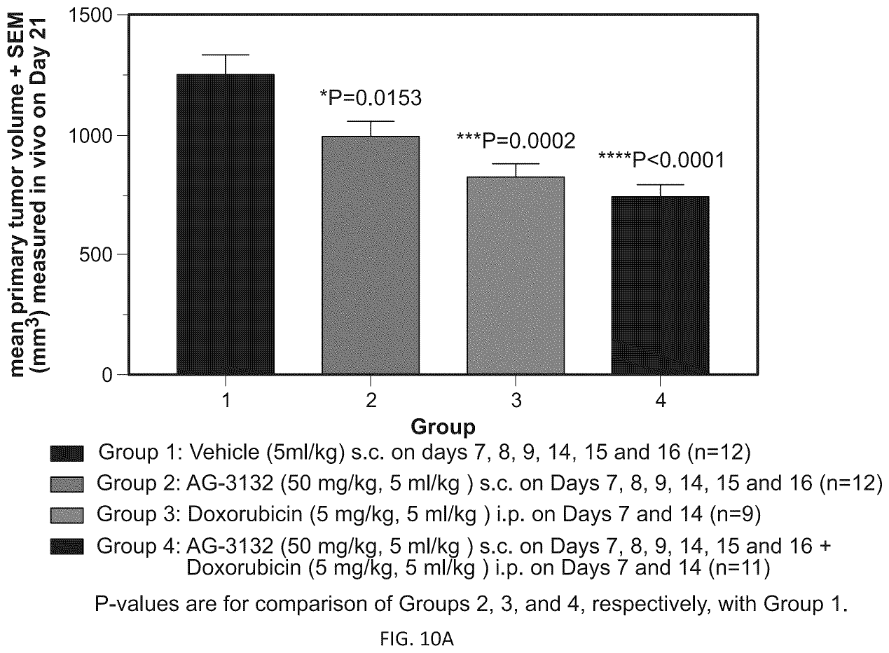

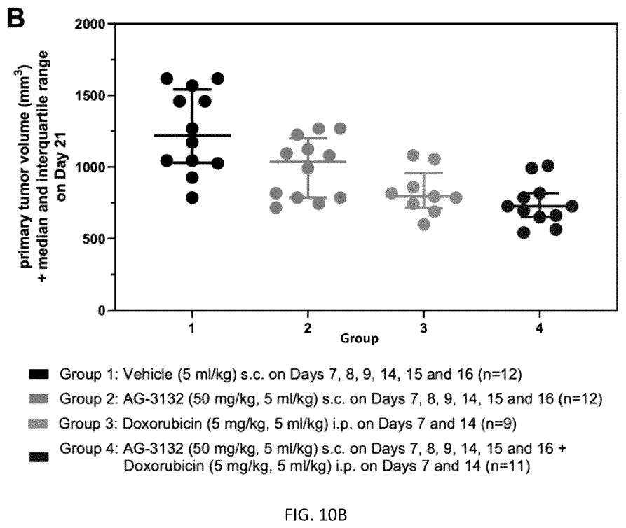

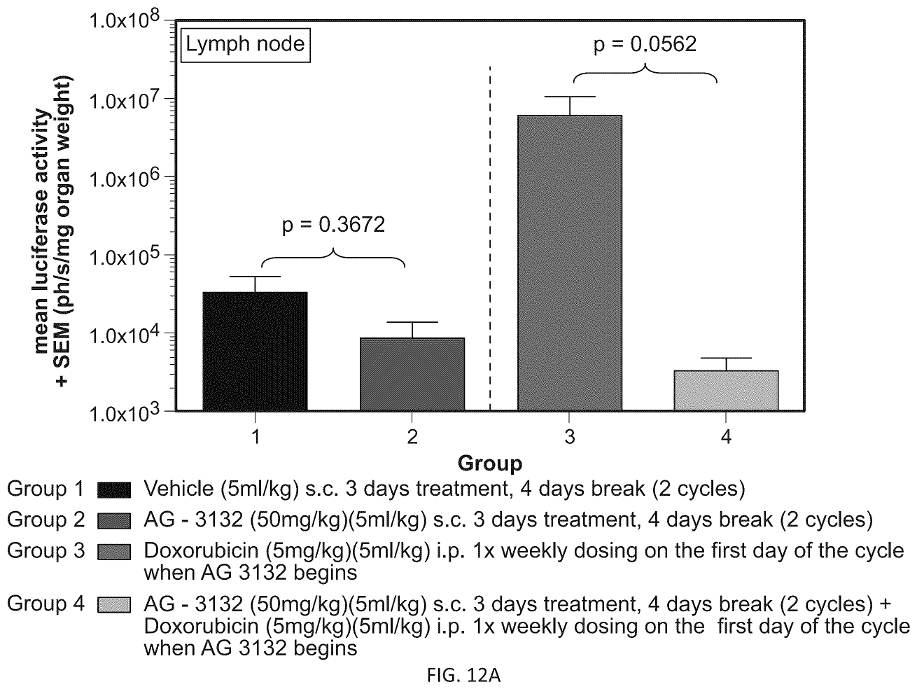

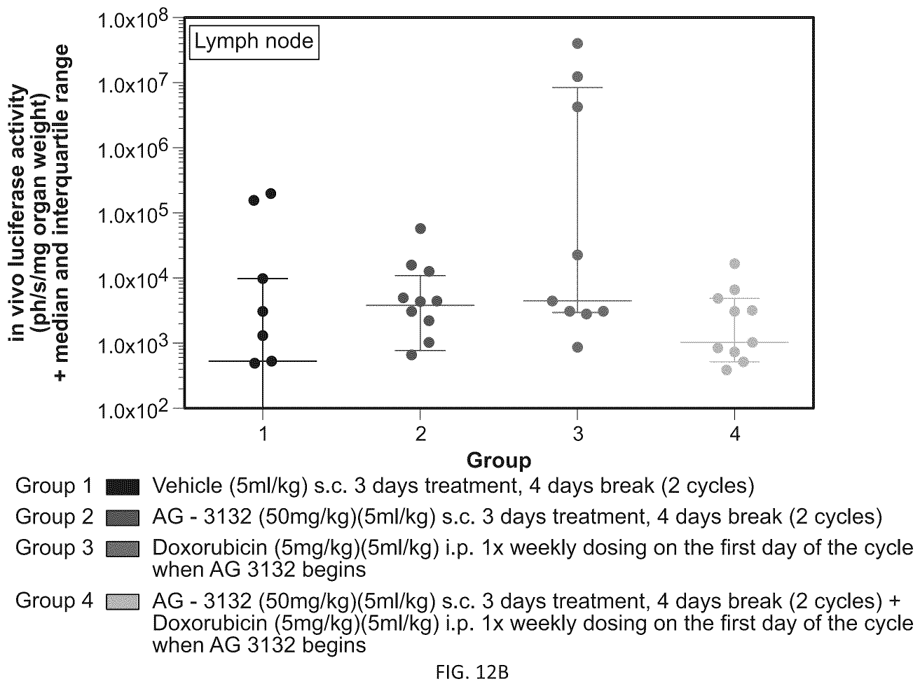

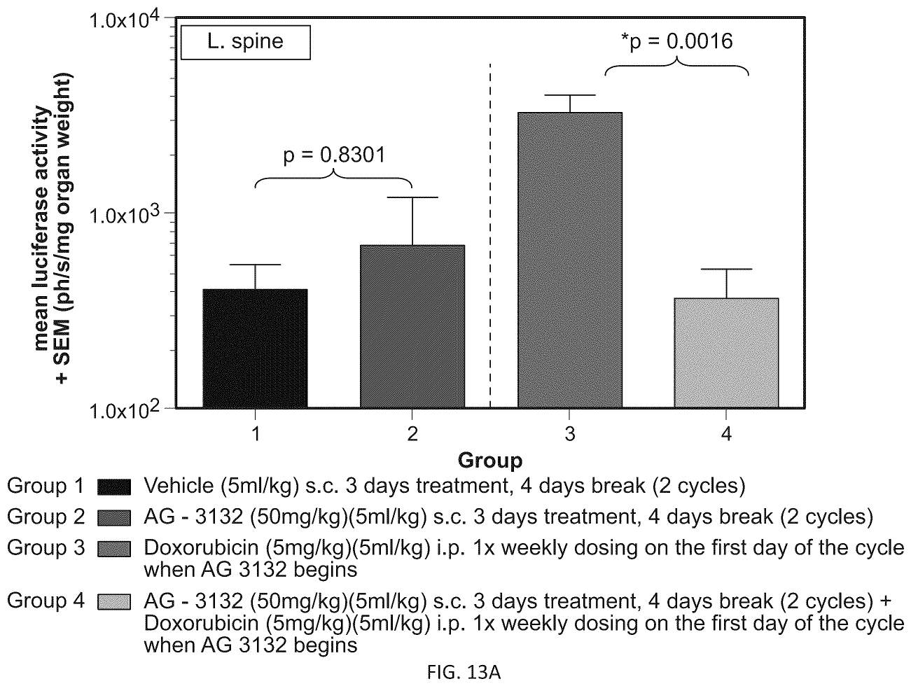

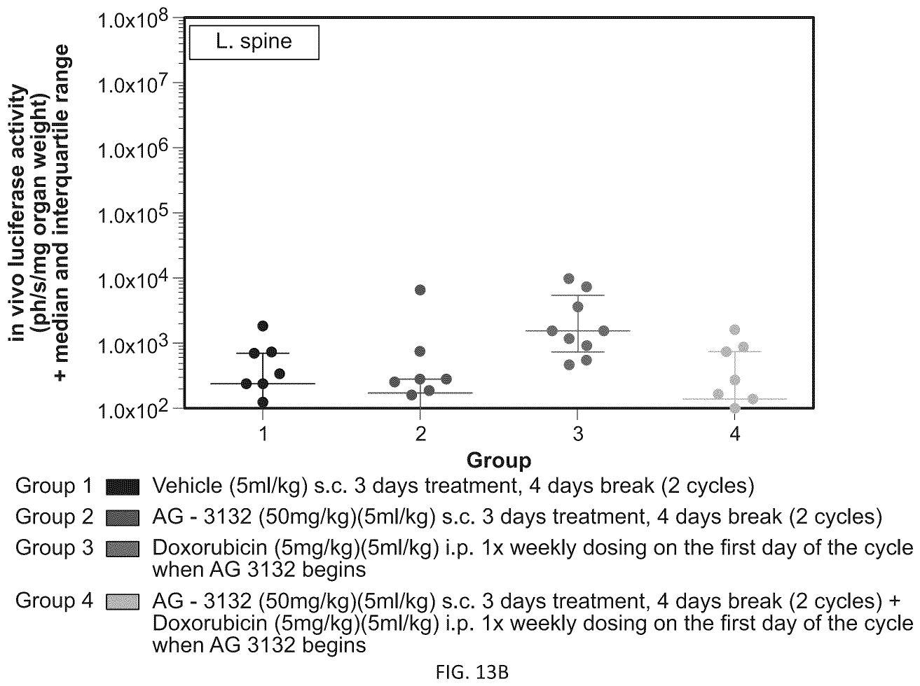

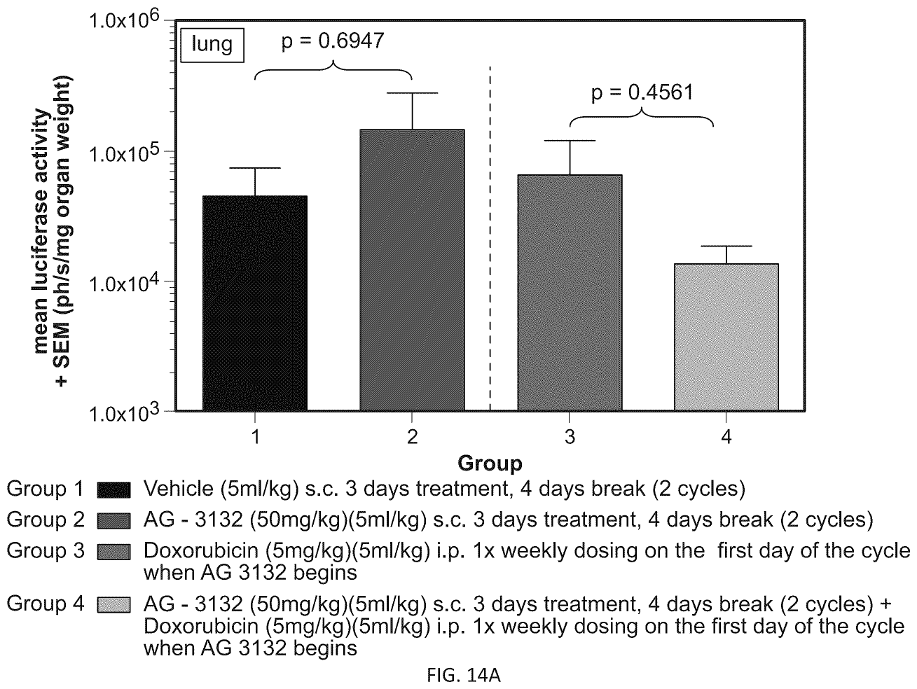

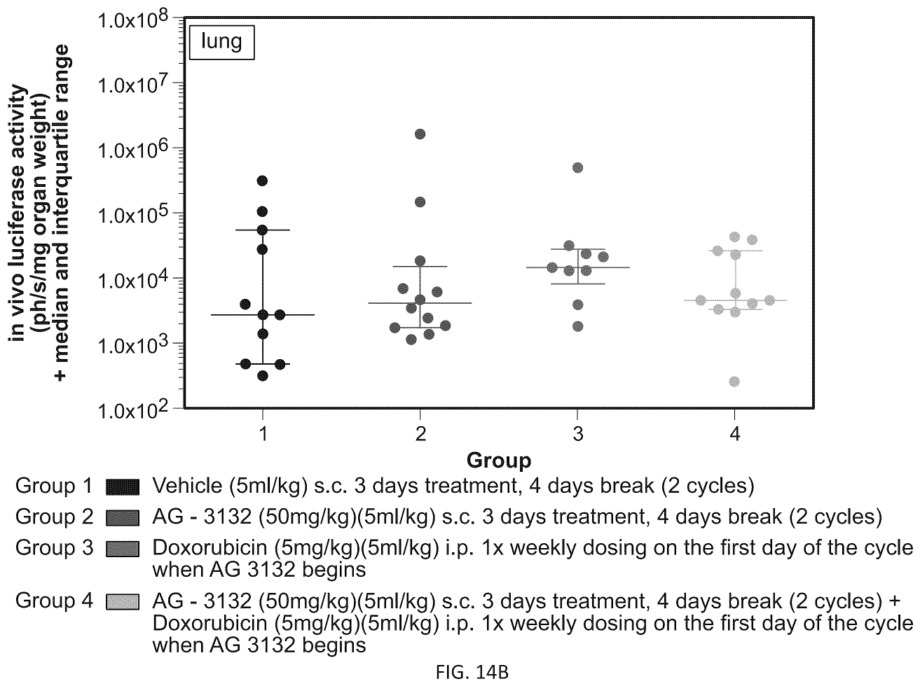

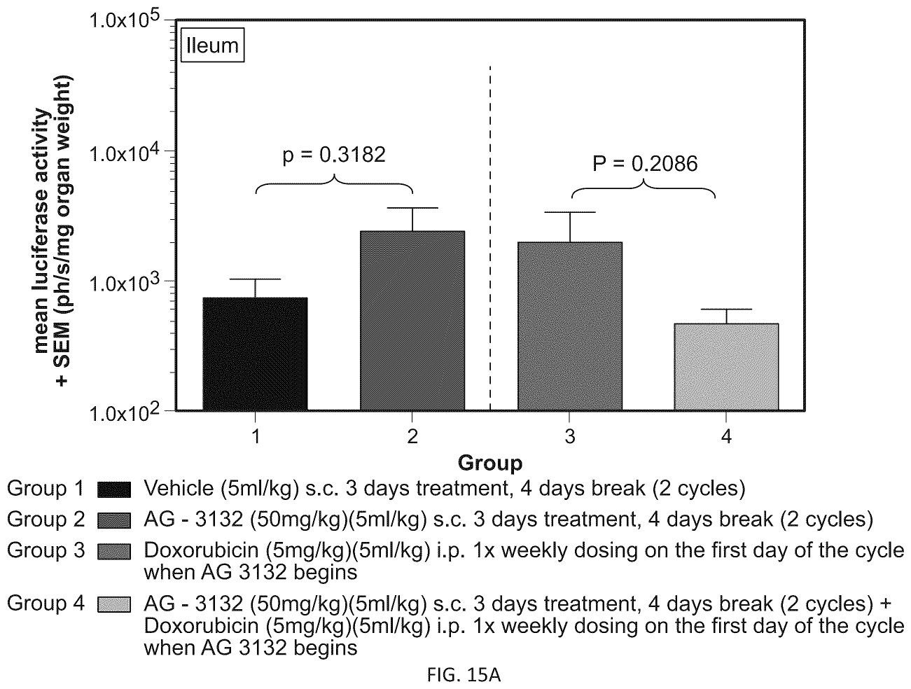

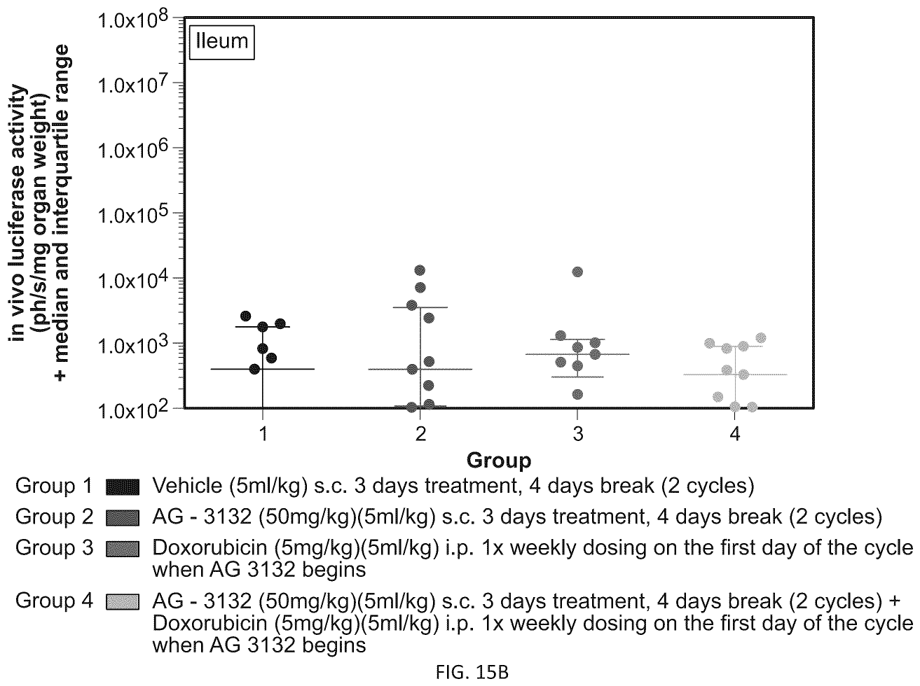

In summary, statistically significant primary tumor growth inhibition was observed for all treatment groups compared to vehicle control (Group 1), as measured in vivo on Day 21, when at least 75% of animals from Groups 3 and 4 were still alive (FIGS. 9, 10A, and 10B). The analysis of metastases to lung, lumbar spine, ileum and lymph nodes, collected during necropsies, using an ex vivo luciferase assay revealed a significant decrease of the luciferase signals of lumbar spine metastases in mice administered AG-3132+doxorubicin (Group 4) compared to mice administered doxorubicin alone (Group 3) (FIGS. 12A-15B).

### Animal Weight

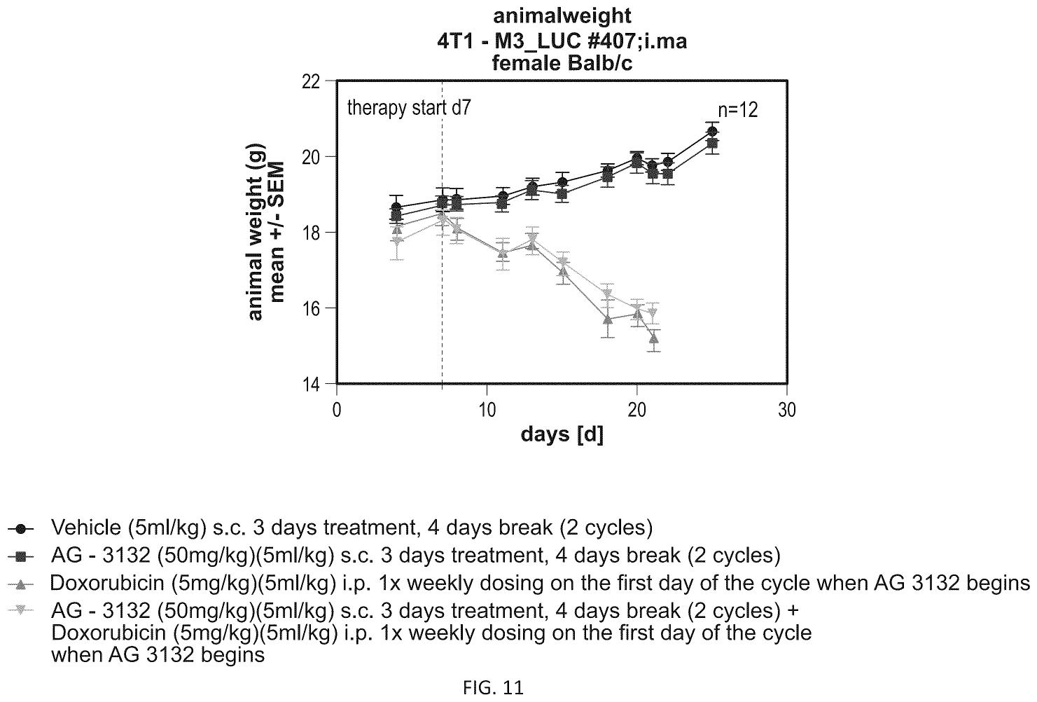

Mean animal weights of Groups 3 and 4 (treated with doxorubicin) continuously decreased throughout the study, until both groups were terminated on Day 21, after three animals of Group 3 and one animal of Group 4 had to be euthanized due to excessive weight loss. Mean animal weights of Group 1 and 2 continuously increased until end of study on Day 25 (FIG. 11).

### In Vivo Primary Tumor Growth

During the course of the study, the growth of the 4T1-M3_Luc primary tumors, which had been implanted into the mammary fat pad, was determined twice weekly by caliper measurement (FIG. 9).

A statistically significant tumor growth inhibition was observed for all treatment groups compared to vehicle control (Group 1), as measured in vivo on Day 21, when at least 75% of animals from Groups 3 and 4 were still alive (FIGS. 10A and 10B).

### Necropsy Findings and Sample Collection

During necropsies on Day 21 (for Groups 3 and 4) and Day 25 (final necropsy for Groups 1 and 2), primary tumors were collected and tumor volumes and wet weights determined. All primary tumors collected were processed as snap-frozen samples. Additionally, all organ samples (lung, lumbar spine, ileum and pooled axillar and inguinal lymph nodes) were collected successfully, weighed (with exception of lymph nodes), and analyzed with regard to metastasis pattern using an ex vivo luciferase assay. Furthermore, terminal blood samples were taken and used for EDTA-plasma preparation.

Test animals

| | |
|---|---|
| Species: | Mus musculus |
| Strain: | BALB/c (BALB/cAnNCrl) |
| Sex: | Female |
| Source: | Charles River GmbH |
| | Sandhofer Weg 7 |
| | 97633 Sulzfeld Germany |
| Number of animals: | 62 (48 after randomization) |
| Age at delivery: | 5 weeks |

Cell culture

| | |
|---|---|
| Cell Line: | 4T1-M3_Luc |
| CPQ (RBE | #407 |
| identifier): | |
| Origin: | Originated from murine breast adenocarcinoma |
| Modification: | Parental 4T1 cells (CPQ-289) were transduced using |
| | a plasmid encoding the luciferase protein under |
| | Neomycin (G418) resistance (1 mg/ml) and thereafter |
| | in vivo passaged to generate a more metastasizing |
| | cell population. |
| Incubation: | 37° C., 5% CO2 |
| Medium: | RPMI-1640 high Glutamax 1 with 10% FCS, 100 units |
| | penicillin/ml, and 100 μg of streptomycin/ml |
| Expansion: | 70%-90% confluent cultures are split routinely using |
| | trypsin/EDTA |
| Quality control: | Routine cell line authentication by a third party, as |
| | well as in-house mycoplasma testing using PCR. |
| Passage number: | At cell culture start: p0 |
| | At implantation day: p4, viability: 95% |

### Tumor Implantation

On Day 0, 4T1-M3_Luc tumor cells (0.5×106 cells in 100 μl PBS) were implanted into the left mammary fat pad of each mouse.

### Tumor Growth Monitoring

Primary tumor volumes were determined by caliper measurement. Tumor sizes were calculated according to the formula W²×L/2 (L=length and W=the perpendicular width of the tumor, L>W).

### Randomization

When a mean tumor volume of 84.4 mm³ was reached in a cohort of tumor-bearing mice, these tumor-bearing animals were block-randomized. For block-randomization, a robust automated random number generation within individual blocks was used (MS-Excel 2016).

### Treatment

Mice were treated according to the following dosing schedule. Treatment started on Day 7, the day of randomization.

TABLE 5
Dosing schedule

| | | | Dose | | | | Number |
|---|---|---|---|---|---|---|---|
| | Test | Dose | Volume | Administration | Dosing | | of |
| Group | specimen | (mg/kg) | (ml/kg) | route | frequency | Animal ID¹⁾ | animals |
| 1 | Vehicle | — | 5 | s.c. | daily on Days 7, 8, | 2, 4, 9, 13, | 12 |
| | | | | | 9, 14, 15 and 16 | 18, 28, 40, | |
| | | | | | | 46, 47, 51, | |
| | | | | | | 58, 62 | |
| 2 | AG-3132 | 50 | 5 | s.c. | daily on Days 7, 8, | 7, 22, 27, | 12 |
| | | | | | 9, 14, 15 and 16 | 33, 35, 37, | |
| | | | | | | 38, 41, 44, | |
| | | | | | | 54, 55, 61 | |
| 3 | Doxorubicin | 5 | 5 | i.p | daily on Days 7 | 3, 10, 14, | 12 |
| | | | | | and 14 | 20, 21, 23, | |
| | | | | | | 30, 31, 32, | |
| | | | | | | 39, 57, 59 | |
| 4 | AG-3132 + | 50 + | 5 + | s.c. + | daily on Days 7, 8, | 1, 6, 11, 12, | 12 |
| | Doxorubicin | 5 | 5 | i.p. | 9, 14, 15 and 16 + | 24, 26, 34, | |
| | | | | | daily on Days 7 | 36, 45, 53, | |
| | | | | | and 14 ²⁾ | 56, 60 | |

¹⁾All animals listed here share the common suffix/22;
²⁾ Administered 2 hours prior to AG-3132.

### Termination

During the course of the study, individual animals were euthanized due to ethical abortion criteria without performing a necropsy. No animal was found dead prior to study end. After three animals of Group 3 and one animal of Group 4 had to be euthanized due to excessive weight loss, both Groups were terminated on Day 21 and a necropsy was performed on the terminated animals. After one animal of Group 1 was euthanized due ethical abortion criteria on Day 22 and four more animals of Group 1 reached ethical abortion criteria on Day 25, the study was terminated, all remaining animals were sacrificed, and a necropsy was performed.

### Necropsy

At necropsies, animals were weighed, and a final in vivo tumor volume measurement was performed. Thereafter, animals were anesthetized by isoflurane for blood sampling and subsequently euthanized by cervical dislocation. Tumors and selected organs (lung, axillar and inguinal lymph nodes, lumbar spine and ileum) were collected for further processing.

### Blood Sampling

At necropsy, animals were anesthetized by isoflurane and blood was taken with micro capillaries via retro-orbital vein puncture slightly rotated (terminal blood sampling, collection of ˜400-500 μl full blood), and immediately transferred into EDTA coated tubes (K₂E tubes) on ice. To obtain EDTA-plasma, tubes were centrifuged at 4° C. for 10 min at 6800 g. After centrifugation, the supernatant was transferred to a new polypropylene tube labeled with study and animal number (label is temperature-resistant −80 to +100° C., not adherent in liquid nitrogen) and stored at −80° C. until further use.

### Tumor Tissue and Organ Collection (Snap-Frozen Sampling)

Primary tumor tissues were collected, and wet weight and tumor volume determined. Thereafter, tumor tissues were snap-frozen in liquid nitrogen, transferred to polypropylene tubes labeled with the study and animal number (label is temperature-resistant −80 to +100° C., not adherent in liquid nitrogen) and stored appropriately at −80° C.

The selected organs lung, axillar and inguinal lymph nodes, lumbar spine and ileum were collected at necropsy. All collected samples were weighed (three decimal places of gram, no weighing of lymph nodes) and used for metastases pattern analysis.

### Metastases Pattern: Ex Vivo Luciferase Assay

At necropsy, selected organs (lung, axillar and inguinal lymph nodes (lymph nodes pooled in one tube), lumbar spine and ileum) were collected, weighed, snap-frozen in ex vivo luciferase buffer and stored at −80° C. until analysis. For the ex vivo luciferase assay, all selected organs were homogenized and assayed for luciferase activity using the kit from Promega (#E1501) according to the instructions from the manufacturer. The luciferase activity was read with an Enspire (Perkin Elmer). Organ weights determined during necropsy were used to normalize luciferase activities (except for lymph nodes).

### Statistical Analysis

Data of the individual groups were analyzed using descriptive data analysis (Mean with SEM, Median with interquartile range). Statistical analysis of efficacy data was done using the Mann Whitney test, the unpaired Student's t-test

and the One-way ANOVA with Dunnett's post-test. All data analysis was performed using GraphPad Prism 9 from GraphPad Software, Inc., San Diego, USA.

Abbreviations

| | |
|---|---|
| approx. | approximately |
| BLI | bioluminescence imaging |
| CD | cluster of differentiation |
| CoA | Certificate of analysis |
| d | day |
| DMEM | Dulbecco's Modified Eagle Medium |
| EDTA | ethylenediaminetetraacetic acid |
| FCS | fetal calf serum |
| FELASA | Federation of Laboratory Animal Science Associations |
| FFPE | formalin-fixed, paraffin-embedded |
| GV-SOLAS | Gesellschaft fur Versuchstierkunde/Society of Laboratory |
| | Animal Science |
| i.p | intraperitoneal |
| i.v. | intravenous |
| IVC | individually ventilated cage |
| i.t. | intratumoral |
| i. ma. | intramammary |
| max. | maximal |
| min | minute |
| MS | Microsoft |
| n.a. | not applicable |
| PCR | polymerase chain reaction |
| PD | pharmacodynamic |
| PK | pharmacokinetic |
| PBS | phosphate buffered saline |
| p.o. | per os |
| rpm | revolutions per minute |
| RPMI-1640 | Roswell Park Memorial Institute-1640 medium |
| RT | room temperature |
| s.c. | subcutaneous |
| SEM | standard error of mean |
| tbd | to be determined |

Synthesis of and additional information on compounds disclosed herein can be found in International Application Pub No. WO2019/051269 and International Application Pub No. WO2020/160333, the contents of each of which are incorporated by reference herein in their entirety for all purposes.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the following.

### Additional Embodiments

Notwithstanding the appended claims, the disclosure set forth herein is also described by the following clauses.

Clause 1. A method of treating, reducing, or preventing metastasis of cancer in a subject in need thereof, comprising:
Administering to the subject a therapeutically effective amount of a composition comprising
an ectonucleotide pyrophosphatase/phosphodiesterase 1 (ENPP1) inhibitor.
   Clause 2. The method of clause 1, further comprising:
administering to the subject a therapeutically effective amount of a composition comprising
an immune checkpoint inhibitor in combination with the composition comprising
an ectonucleotide pyrophosphatase/phosphodiesterase 1 (ENPP1) inhibitor.
   Clause 3. The method of clause 1 or 2, further comprising:
administering to the subject a therapeutically effective amount of a composition comprising a chemotherapeutic agent in combination with the composition comprising an ectonucleotide pyrophosphatase/phosphodiesterase 1 (ENPP1) inhibitor.
   Clause 4. The method of any one of clauses 1-3, further comprising:
administering radiation therapy to the subject.
   Clause 5. The method of any one of clauses 1-4, wherein the ENPP1 inhibitor is of the formula (I):

Y-A-L-X (I)
wherein:
Y is selected from the group consisting of an aryl, a substituted aryl, a heteroaryl, a substituted heteroaryl, a carbocycle, a substituted carbocycle, a heterocycle and a substituted heterocycle;
A is selected from the group consisting of a carbocycle, a substituted carbocycle, a heterocycle and a substituted heterocycle;
L is a covalent bond or a linker; and
X is a hydrophilic head group,
or a pro-drug, pharmaceutically acceptable salt or solvate thereof.
   Clause 6. The method of clause 5, wherein the hydrophilic head group (X) is selected from boronic acid, hydroxamic acid, phosphonic acid, phosphonate, phosphonate ester, phosphate, phosphate ester, thiophosphate, thiophosphate ester, phosphoramidate, thiophosphoramidate, sulfonic acid, sulfonate, sulfate, hydroxamic acid, and carboxylic acid.
   Clause 7. The method of clause 6, wherein the hydrophilic head group (X) is selected from phosphonic acid, phosphonate, phosphonate ester, phosphate, phosphate ester, thiophosphate, thiophosphate ester, phosphoramidate and thiophosphoramidate.
   Clause 8. The method of any one of clauses 1-7, wherein L-X comprises a group of formula (XI):

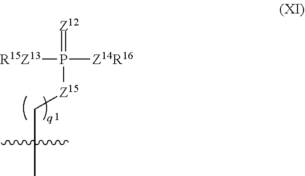

wherein:
Z¹² is selected from O and S;
Z¹³ and Z¹⁴ are each independently selected from O and NR′ wherein R′ is H, alkyl or substituted alkyl;
Z¹⁵ is selected from O and CH₂;
R¹⁵ and R¹⁶ are each independently selected from H, alkyl, substituted alkyl, alkenyl, substituted alkenyl, alkoxy, substituted alkoxy, aryl, substituted aryl, an acyl group, heterocycle, substituted heterocycle cycloalkyl and substituted cycloalkyl; and
q¹ is an integer from 0 to 6 (e.g., 0-5).
   Clause 9. The method of clause 8, wherein L-X is selected from:

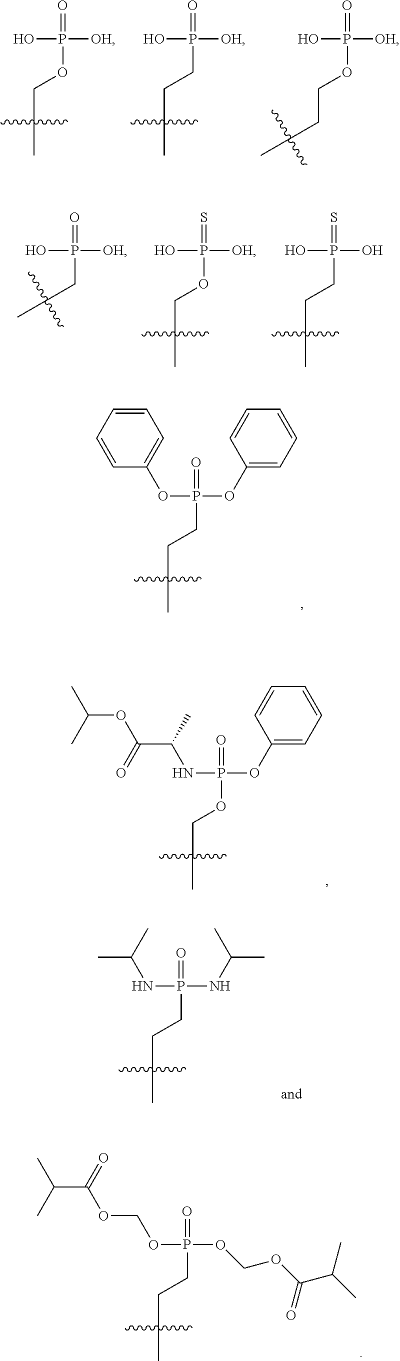

Clause 10. The method of any one of clauses 1-9, wherein X is phosphonic acid or phosphonate ester.
Clause 11. The method of clause 1, wherein L-X comprises a group of the formula (XII):

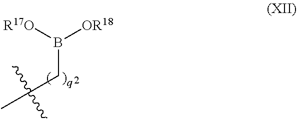

wherein:
R¹⁷ and R¹⁸ are each independently selected from H, alkyl, substituted alkyl, alkenyl, substituted alkenyl, alkoxy, substituted alkoxy, aryl, substituted aryl, an acyl group, heterocycle, substituted heterocycle cycloalkyl and substituted cycloalkyl or R¹⁷ and R¹⁸ together with the atoms to which they are attached form a ring selected from heterocycle and substituted heterocycle; and
q² is an integer from 1 to 6.
   Clause 12. The method of clause 11, wherein L-X is of the structure:

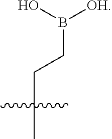 [SMILES: *CCB(O)O]

Clause 13. The method of clause 5, wherein L-X comprises a group of formula (XIII):

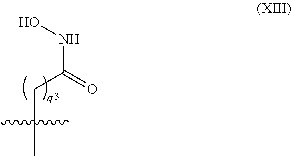

wherein q3 is an integer from 1 to 6.
   Clause 14. The method of clause 13, wherein L-X is selected from:

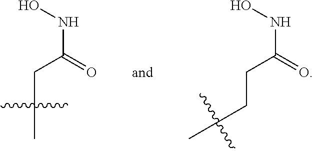 [SMILES: *CC(=O)NO ; "and" ; *CCC(=O)NO]

Clause 15. The method of clause 5, wherein L-X comprises a group of formula (XIV):

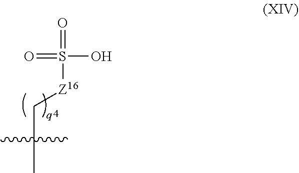

wherein: Z¹⁶ is selected from O and CH₂; and q⁴ is an integer from 0 to 6.
   Clause 16. The method of clause 15, wherein L-X is selected from:

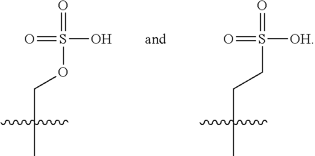 [SMILES: *COS(=O)(=O)O ; "and" ; *CCS(=O)(=O)O]

Clause 17. The method of clause 5, wherein L-X comprises a group of formula (XV):

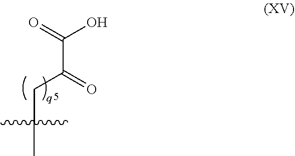

wherein q⁵ is an integer from 1 to 6.
   Clause 18. The method of clause 17, wherein L-X is selected from:

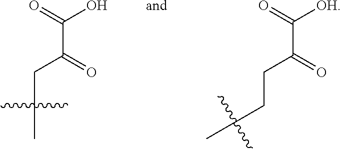 [SMILES: *CC(=O)C(=O)O ; "and" ; *CCC(=O)C(=O)O]

Clause 19. The method of clause 5, wherein L-X comprises a group of formula (XVI):

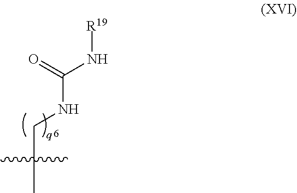

wherein:
R¹⁹ is selected from H, alkyl, substituted alkyl, alkenyl, substituted alkenyl, alkoxy, substituted alkoxy, aryl, substituted aryl, an acyl group, heterocycle, substituted heterocycle cycloalkyl and substituted cycloalkyl; and
q⁶ is an integer from 1 to 6.
   Clause 20. The method of clause 19, wherein L-X is of the structure:

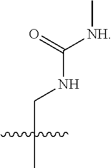 [SMILES: *CNC(=O)NC]

Clause 21. The method of clause 5, wherein L-X comprises a group of formula (XVII):

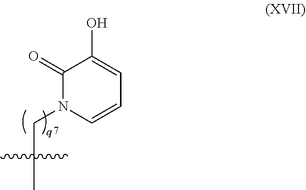

wherein q7 is an integer from 1 to 6.
   Clause 22. The method of clause 21, wherein L-X is of the structure:

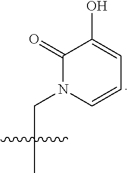 [SMILES: *Cn1cccc(O)c1=O]

Clause 23. The method of any one of clauses 5-22, wherein A is a heterocycle or substituted heterocycle.
Clause 24. The method of clause 23, wherein A is selected from piperidine, substituted piperidine, piperazine and substituted piperazine.
Clause 25. The method of any one of clauses 23-24, wherein A is:

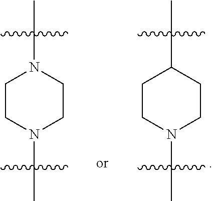 [SMILES: *N1CCN(*)CC1 ; *C1CCN(*)CC1]

Clause 26. The method of any one of clauses 5-22, wherein A is a carbocycle (e.g., a 5-, 6- or 7-membered monocyclic carbocycle).
Clause 27. The method of clause 26, wherein A is a cycloalkyl or substituted cycloalkyl.
Clause 28. The method of clause 27, wherein A is:

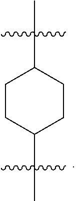 [SMILES: *C1CCC(*)CC1]

Clause 29. The method of clause 26, wherein A is aryl or substituted aryl.
Clause 30. The method of clause 29, wherein A is phenylene or substituted phenylene.
Clause 31. The method of clause 30, wherein A is:

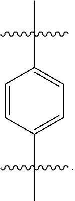 [SMILES: *c1ccc(*)cc1]

Clause 32. The method of any one of clauses 5-31, wherein L is a linear linker having a backbone of 1 to 12 atoms in length and comprising one or more groups selected from alkylene, substituted alkylene, —CO—, —O—, —NR′— —NR′CO—, —CO₂— and —NR′CO₂— wherein R′ is H, alkyl or substituted alkyl.
Clause 33. The method of clause 32, wherein L is —(CH₂)n-, and n is an integer from 1 to 6 (e.g., 1, 2, 3, 4, 5 or 6).
Clause 34. The method of clause 33, wherein n is 1 or 2.
Clause 35. The method of any one of clauses 5-34, wherein Y is selected from quinazoline, substituted quinazoline, quinoline, substituted quinoline, naphthalene, substituted naphthalene, isoquinoline, substituted isoquinoline, 7H-purine, substituted 7H-purine, pyrimidine, substituted pyrimidine.
Clause 36. The method of any one of clauses 5-34, wherein Y is selected from 4-quinazolinyl, substituted 4-quinazolinyl, 4-quinolinyl, substituted 4-quinolinyl, 1-naphthalyl, substituted 1-naphthalyl, 4-isoquinolinyl, substituted 4-isoquinolinyl, 6-(7H-purinyl), substituted 6-(7H-purinyl), 4-pyrimidinyl, substituted 4-pyrimidinyl.
Clause 37. The method of any one of clauses 35-36, wherein Y is a group of the formula:

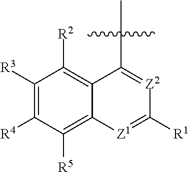

wherein:
Z¹ and Z² are each independently selected from CR¹ and N;
each R¹ is independently selected from H, cyano, trifluoromethyl, halogen, amino, alkyl, substituted alkyl, alkenyl, substituted alkenyl, heterocycle and substituted heterocycle;
R² and R⁵ are each independently selected from the group consisting of H, OH, alkyl, substituted alkyl, alkenyl, substituted alkenyl, alkoxy, substituted alkoxy, —OCF₃, halogen, amine, substituted amine, amide, heterocycle and substituted heterocycle;
R³ and R⁴ are each independently selected from the group consisting of H, OH, alkyl, substituted alkyl, alkenyl, substituted alkenyl, alkoxy, substituted alkoxy, —OCF₃, halogen, amine, substituted amine, amide, heterocycle and substituted heterocycle;
or R³ and R⁴ together with the carbon atoms to which they are attached form a fused selected from heterocycle, substituted heterocycle, cycloalkyl, substituted cycloalkyl, aryl and substituted aryl.
   Clause 38. The method of clause 37, wherein the ENPP1 inhibitor comprises the formula:

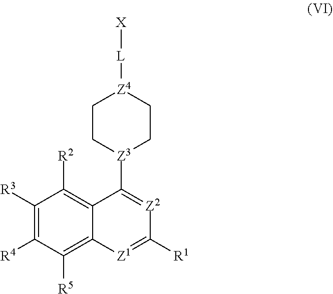

wherein,
L is selected from —CH₂—, —(CH₂)₂—, —(CH₂)₃—, —(CH₂)₄—, —(CH₂)₅— and —(CH₂)₆—;
X is selected from:

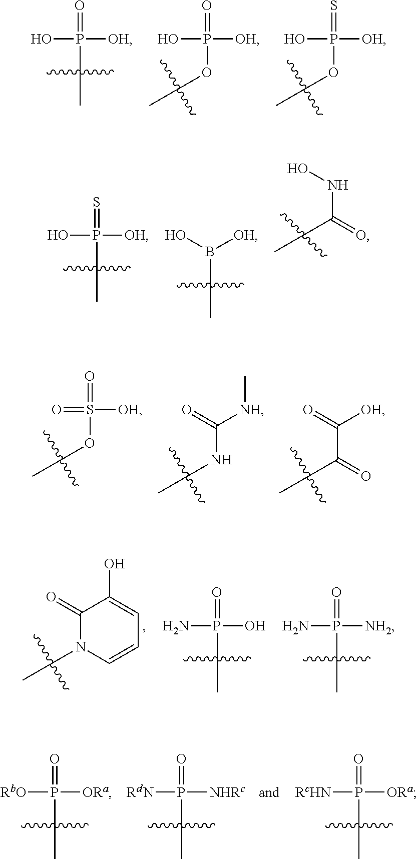

wherein:
R^{a} and R^{b} are each independently selected from aryl, alkyl, —CH₂OC(O)R^{e}, —CH₂OC(O)OR^{e};
R^{c} and R^{d} are each independently selected from —C(CH₃)C(O)OR^{e}, alkyl and wherein R^{e} is alkyl; and
Z³ and Z⁴ are each independently selected from CR and N, wherein R is H, alkyl or substituted alkyl.
   Clause 39. The method of any one of clauses 1-3, wherein the ENPP1 inhibitor comprises the formula (VI):

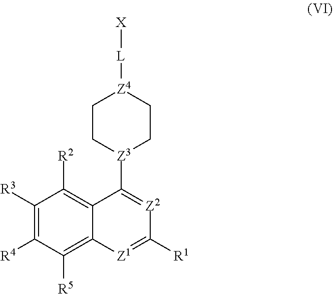

wherein,
X is a hydrophilic head group selected from phosphonic acid, phosphonate, phosphonate ester, phosphate, phosphate ester, thiophosphate, thiophosphate ester, phosphoramidate and thiophosphoramidate;
L is selected from —CH₂—, —(CH₂)₂—, —(CH₂)₃—, —(CH₂)₄—, —(CH₂)₅— and —(CH₂)₆—;
Z¹ and Z² are each independently selected from CR¹ and N;
Z³ and Z⁴ are each independently selected from CR and N, wherein R is H, alkyl or substituted alkyl;
each R¹ is independently selected from H, alkyl, substituted alkyl, alkenyl, substituted alkenyl, heterocycle and substituted heterocycle;
R² and R⁵ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle;
R³ and R⁴ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle; or R³ and R⁴ together with the carbon atoms to which they are attached form a fused ring selected from heterocycle, substituted heterocycle, cycloalkyl, substituted cycloalkyl, aryl and substituted aryl;
or a pharmaceutically acceptable salt or a solvate thereof.
   Clause 40. The method of any one of clauses 1-3, wherein the ENPP1 inhibitor comprises the formula (VI):

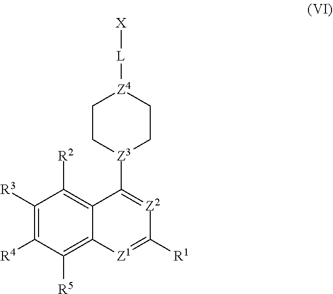

wherein,
X is a hydrophilic head group selected from boronic acid, hydroxamic acid, phosphonic acid, phosphonate, phosphonate ester, phosphate, phosphate ester, thiophosphate, thiophosphate ester, phosphoramidate and thiophosphoramidate;
L is a linker;
Z¹ and Z² are each independently selected from CR¹ and N;
Z³ and Z⁴ are each independently selected from CR and N, wherein R is H, alkyl or substituted alkyl;
each R¹ is independently selected from H, cyano, trifluoromethyl, halogen, amino, alkyl, substituted alkyl, alkenyl, substituted alkenyl, heterocycle and substituted heterocycle;
R² and R⁵ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle;
R³ and R⁴ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle; or R³ and R⁴ together with the carbon atoms to which they are attached form a fused selected from heterocycle, substituted heterocycle, cycloalkyl, substituted cycloalkyl, aryl and substituted aryl;
   or a pro-drug, a pharmaceutically acceptable salt or a solvate thereof
   Clause 41. The method of clause 39 or 40, wherein:
L is selected from —CH₂—, —(CH₂)₂—, —(CH₂)₃—, —(CH₂)₄—, —(CH₂)₅— and —(CH₂)₆—;
X is selected from:

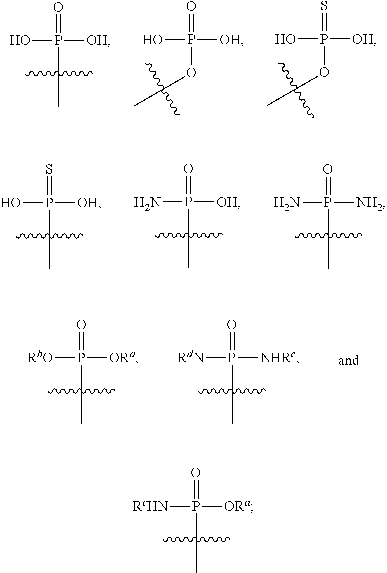

wherein:
R^{a} and R^{b} are each independently selected from aryl, alkyl, —CH₂OC(O)R^{e}, —CH₂OC(O)OR^{e}; and R^{c} and R^{d} are each independently selected from —C(CH₃)C(O)OR^{e}, alkyl and wherein R^{e} is alkyl
   Clause 42. The method of clause 39 or 40, wherein:
L is selected from —CH₂—, —(CH₂)₂—, —(CH₂)₃—, —(CH₂)₄—, —(CH₂)₅— and —(CH₂)₆—;
X is selected from:

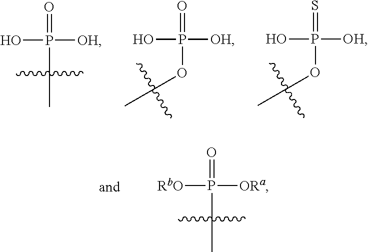

wherein:
R^{a} and R^{b} are each independently selected from aryl, alkyl, —CH₂OC(O)R^{e}, and —CH₂OC(O)OR^{e}; and
wherein R^{e} is alkyl.
   Clause 43. The method of any one of clauses 38-40, of the formula:

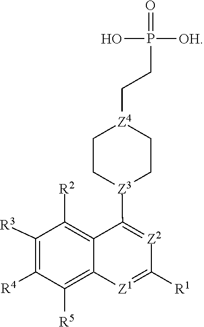

Clause 44. The method of clause 43, wherein the ENPP1 inhibitor is of the formula:

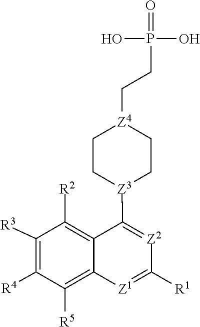

wherein,
Z¹ and Z² are each N;
Z³ is N; and
Z⁴ is CH or N.
   Clause 45. The method of any one of clauses 38-44, wherein the portion of the ENPP1 inhibitor represented by

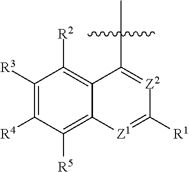

is selected from:

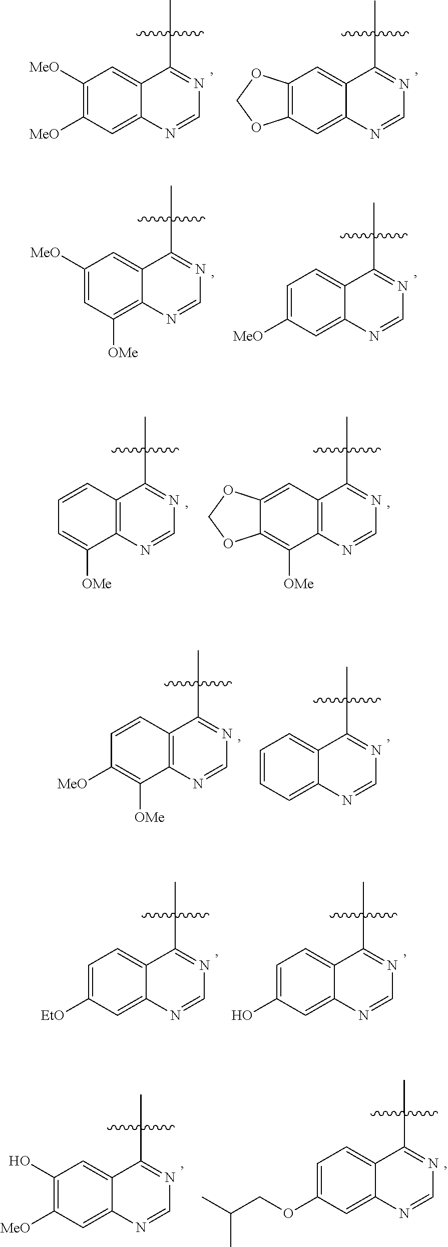

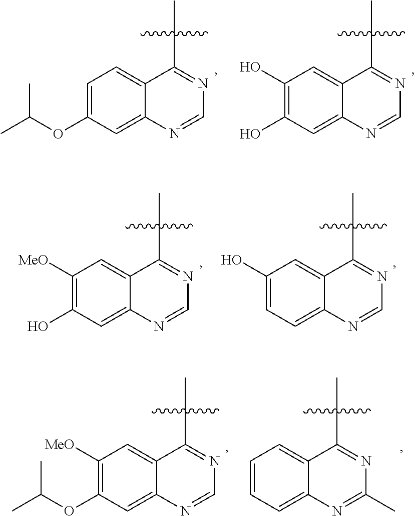

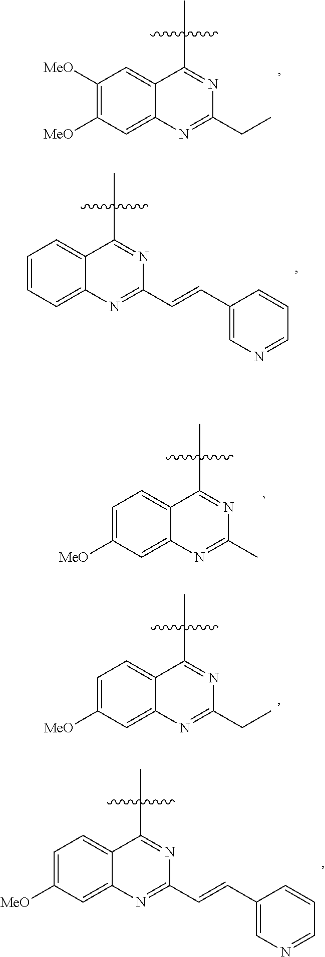 [SMILES: *c1nc(CC)nc2cc(OC)c(OC)cc12 ; *c1nc(/C=C/c2cccnc2)nc2ccccc12 ; *c1nc(C)nc2cc(OC)ccc12 ; *c1nc(CC)nc2cc(OC)ccc12 ; *c1nc(/C=C/c2cccnc2)nc2cc(OC)ccc12]

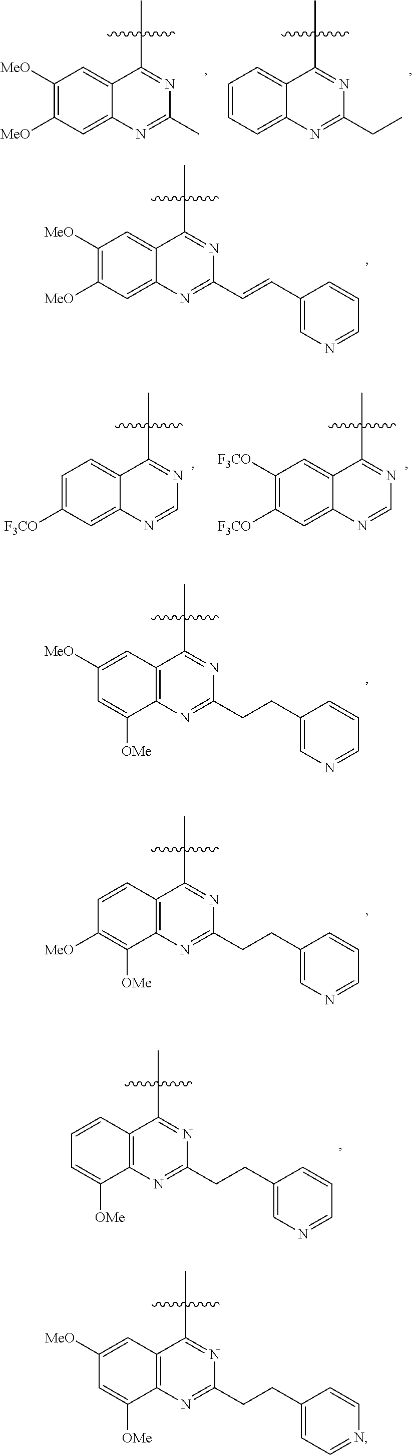

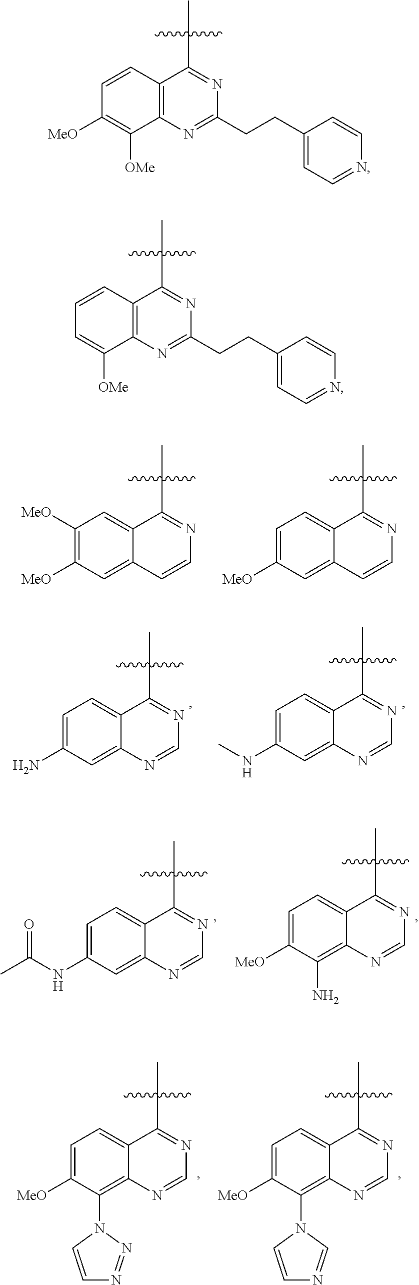

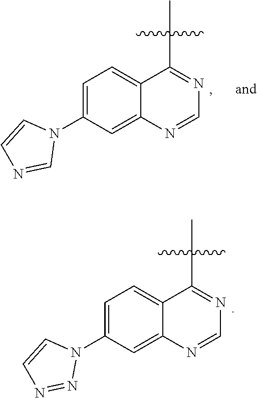 [SMILES: *c1ncnc2cc(-n3ccnc3)ccc12 ; *c1ncnc2cc(-n3ccnn3)ccc12]

Clause 46. The method of clause 37, wherein the ENPP1 inhibitor is of the formula:

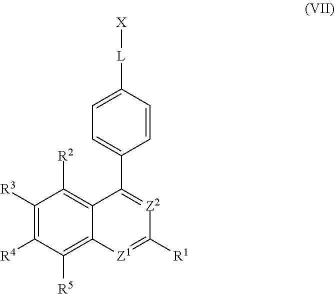

wherein,
L is selected from —CH₂—, —(CH₂)₂—, —(CH₂)₃—, —(CH₂)₄—, —(CH₂)₅— and —(CH₂)₆—;
X is selected from:

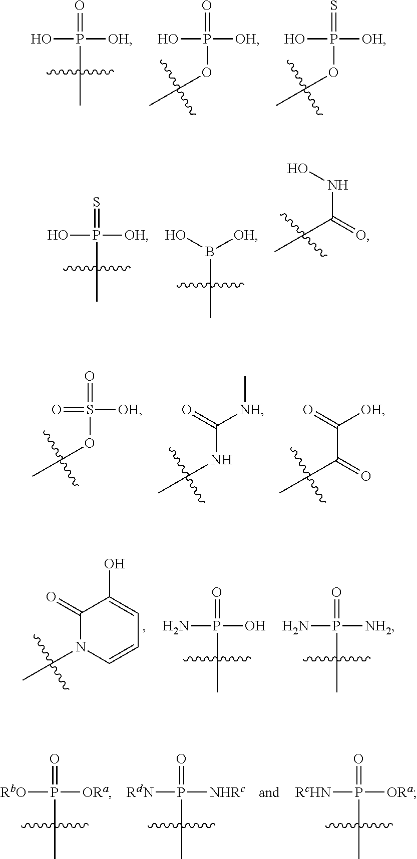

wherein:
R^{a} and R^{b} are each independently selected from aryl, alkyl, —CH₂OC(O)R^{e}, —CH₂OC(O)OR^{e}; and
R^{c} and R^{d} are each independently selected from —C(CH₃)C(O)OR^{e}, alkyl and wherein R^{e} is alkyl.
   Clause 47: The method of any one of clauses 37, 38, 43, and 46, wherein,
R¹ is selected from hydrogen, C₁₋₅ alkyl and vinyl heterocycle;
R² and R⁵ are each independently selected from hydrogen, C₁₋₅ alkyl, amine, triazole, imidazole, amide, alkoxy, OCF₃, halogen and hydroxy; and
R³ and R⁴ are each independently selected from hydrogen, C₁₋₅ alkyl, triazole, imidazole, amine, amide, alkoxy, OCF₃, halogen and hydroxy, or R³ and R⁴ together with the carbon atoms to which they are attached from a fused heterocycle.
   Clause 48. The method of any one of clauses 33 to 37, wherein Y is a group of the formula:

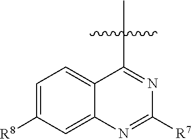

wherein:
R⁷ is selected from H, alkyl, substituted alkyl, alkenyl, substituted alkenyl, heterocycle and substituted heterocycle;
R⁸ is selected from OH, alkyl, substituted alkyl, alkenyl, substituted alkenyl, alkoxy, substituted alkoxy, —OCF₃, halogen, amine, substituted amine, amide, heterocycle and substituted heterocycle.
   Clause 49: The method of clause 48, wherein:
R⁷ is selected from hydrogen, C₁₋₅ alkyl, substituted C₁₋₅ alkyl, vinyl-heterocycle and substituted vinyl-heterocycle; and
R⁸ is selected from hydrogen, C₁₋₅ alkyl, triazole, imidazole, amine, amide, alkoxy, halogen, OCF₃ and hydroxy.
   Clause 50. The method of any one of clauses 37, 38, 43, and 46, wherein Y is a group of the formula:

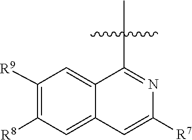

wherein,
R⁷ is selected from H, alkyl, substituted alkyl, alkenyl, substituted alkenyl, heterocycle and substituted heterocycle;
R⁸ and R⁹ are each independently selected from OH, alkyl, substituted alkyl, alkenyl, substituted alkenyl, alkoxy, substituted alkoxy, —OCF₃, halogen, amine, substituted amine, amide, heterocycle and substituted heterocycle; or R⁸ and R⁹ together with the carbon atoms to which they are attached form a fused ring selected from heterocycle, substituted heterocycle, cycloalkyl, substituted cycloalkyl, aryl and substituted aryl.
   Clause 51: The method of clause 50, wherein,
R⁷ is selected from hydrogen, C₁₋₅ alkyl and vinyl heterocycle;
R⁸ and R⁹ are each independently selected from hydrogen, C₁₋₅ alkyl, triazole, imidazole, amine, amide, alkoxy, halogen, OCF₃ and hydroxy, or R⁸ and R⁹ together with the carbon atoms to which they are attached from a fused heterocycle or fused substituted heterocycle.
   Clause 52. The method of any one of clauses 37, 38, 43, and 46, wherein Y is of the formula:

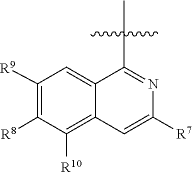

wherein,
R⁷ is selected from H, alkyl, substituted alkyl, alkenyl, substituted alkenyl, heterocycle and substituted heterocycle;
R¹⁰ is selected from OH, alkyl, substituted alkyl, alkenyl, substituted alkenyl, alkoxy, substituted alkoxy, —OCF₃, halogen, amine, substituted amine, amide, heterocycle and substituted heterocycle;
R⁸ and R⁹ are each independently selected from OH, alkyl, substituted alkyl, alkenyl, substituted alkenyl, alkoxy, substituted alkoxy, —OCF₃, halogen, amine, substituted amine, amide, heterocycle and substituted heterocycle; or R⁸ and R⁹ together with the carbon atoms to which they are attached form a fused ring selected from heterocycle, substituted heterocycle, cycloalkyl, substituted cycloalkyl, aryl and substituted aryl.
   Clause 53: The method of clause 52, wherein,
R⁷ is selected from hydrogen, C₁₋₅ alkyl and vinyl heterocycle;
R¹⁰ is selected from hydrogen, C₁₋₅ alkyl, amine, triazole, imidazole, amide, alkoxy, OCF₃ and hydroxy; and
R⁸ and R⁹ are each independently selected from hydrogen, C₁₋₅ alkyl, triazole, imidazole, amine, amide, alkoxy, OCF₃ and hydroxy, or R⁸ and R⁹ together with the carbon atoms to which they are attached from a fused heterocycle or substituted fused heterocycle.
   Clause 54. The method of any one of clauses 37, 38, 43, and 46, wherein Y is of the formula:

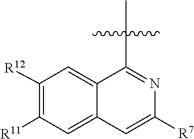

wherein,
R⁷ is selected from H, alkyl, substituted alkyl, alkenyl, substituted alkenyl, heterocycle and substituted heterocycle;
R¹¹ and R¹² are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle; or R¹¹ and R¹² together with the carbon atoms to which they are attached form a fused ring selected from heterocycle, substituted heterocycle, cycloalkyl, substituted cycloalkyl, aryl and substituted aryl.
   Clause 55: The method of clause 54, wherein
R⁷ is selected from hydrogen, C₁₋₅ alkyl, substituted C₁₋₅ alkyl, vinyl-heterocycle and substituted vinyl-heterocycle; and
R¹¹ and R¹² are each independently selected from hydrogen, C₁₋₅ alkyl, triazole, imidazole, amine, amide, alkoxy, halogen, OCF₃ and hydroxy, or R¹¹ and R¹² together with the carbon atoms to which they are attached form a fused heterocycle or substituted fused heterocycle.
   Clause 56. The method of any one of clauses 37, 38, 43, and 46, wherein Y is a group of the formula:

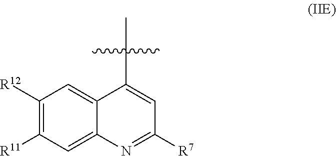

wherein,
R⁷ is selected from the group consisting of H, alkyl, substituted alkyl, alkenyl, substituted alkenyl, heterocycle and substituted heterocycle;
R¹¹ and R¹² are each independently selected from the group consisting of H, OH, alkyl, substituted alkyl, alkenyl, substituted alkenyl, alkoxy, substituted alkoxy, —OCF₃, halogen, amine, substituted amine, amide, heterocycle and substituted heterocycle; or R¹¹ and R¹² together with the carbon atoms to which they are attached form a fused ring selected from heterocycle, substituted heterocycle, cycloalkyl, substituted cycloalkyl, aryl and substituted aryl.
   Clause 57: The method of clause 56, wherein,
R⁷ is selected from hydrogen, C₁₋₅ alkyl, substituted C₁₋₅ alkyl, vinyl-heterocycle and substituted vinyl-heterocycle; and
R¹¹ and R¹² are each independently selected from hydrogen, C₁₋₅ alkyl, triazole, imidazole, amine, amide, alkoxy, halogen, OCF₃ and hydroxy, or R¹¹ and R¹² together with the carbon atoms to which they are attached form a fused heterocycle or substituted fused heterocycle.
   Clause 58. The method of any one of any one of clauses 4-57, wherein Y is selected from:

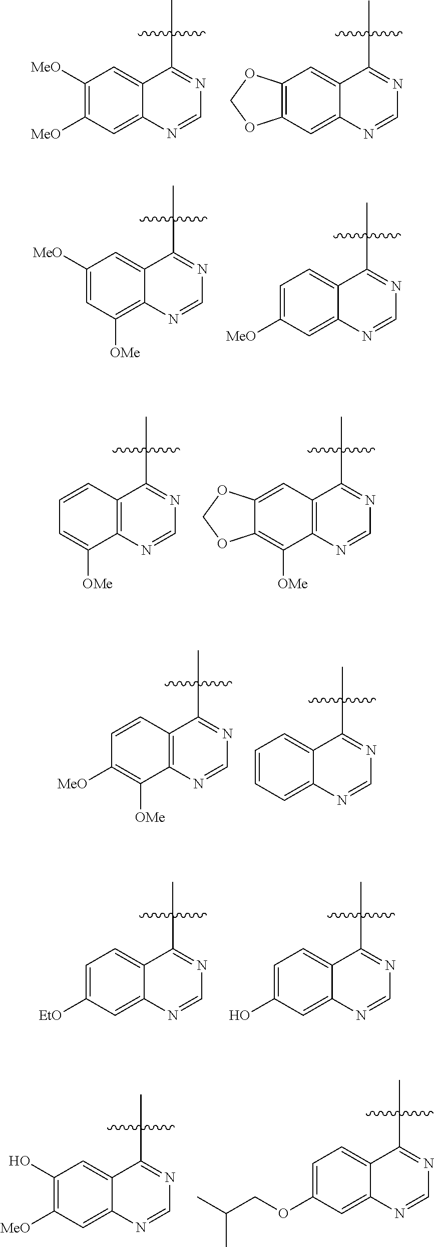

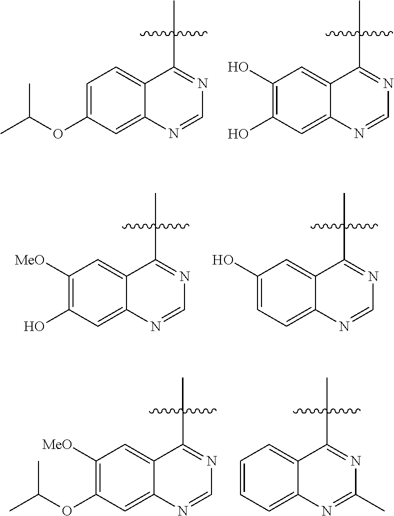

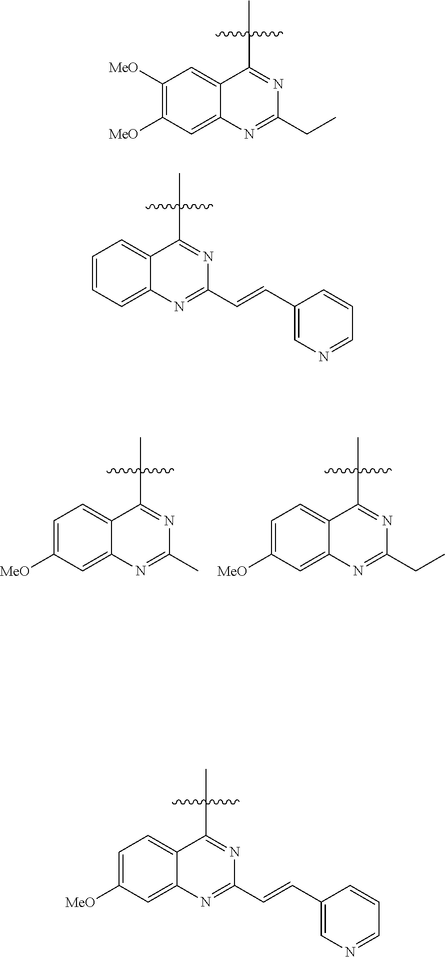

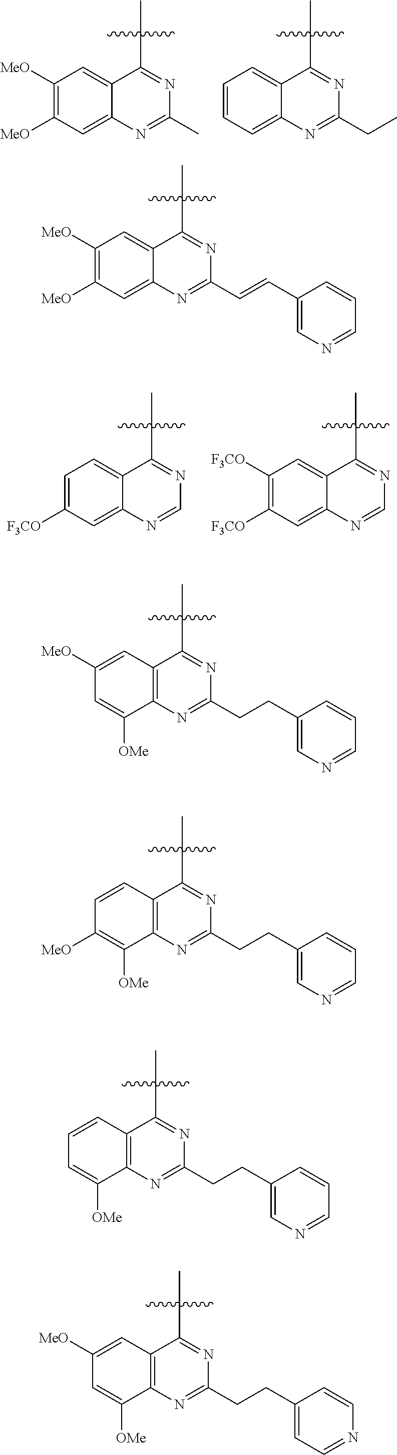

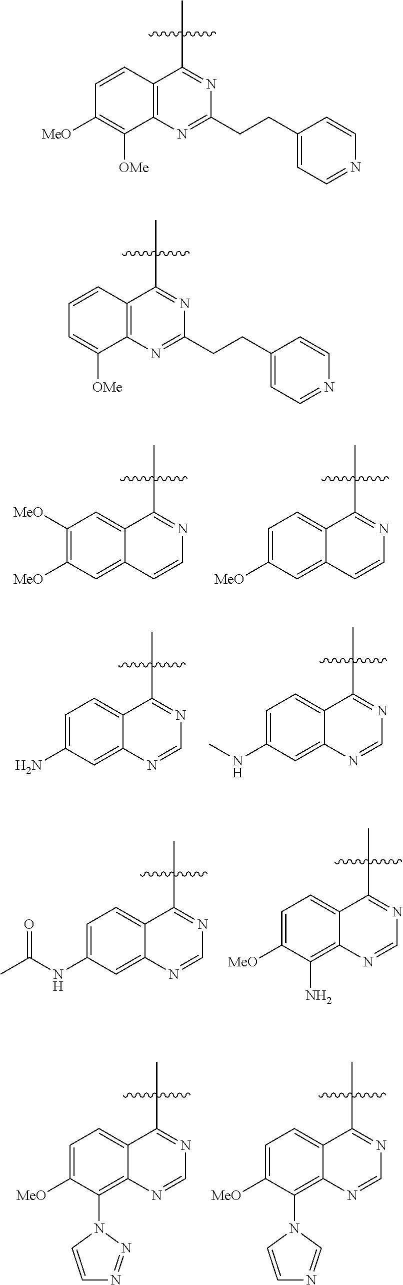

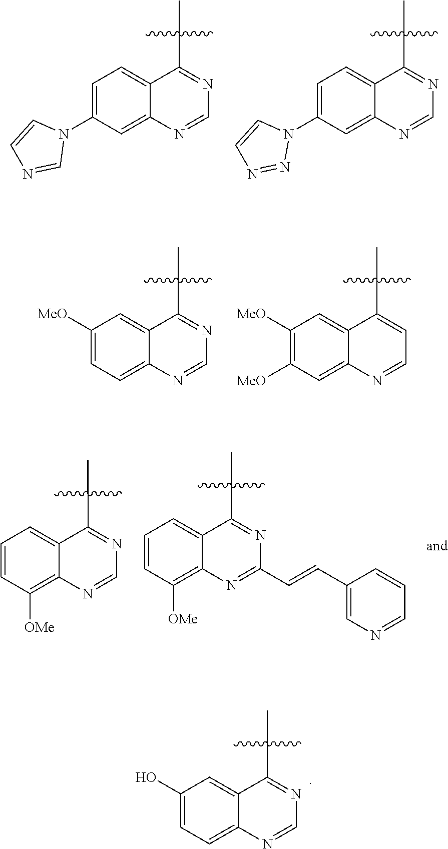

Clause 59. The method of any one of any one of clauses 4-57, wherein Y is selected from:

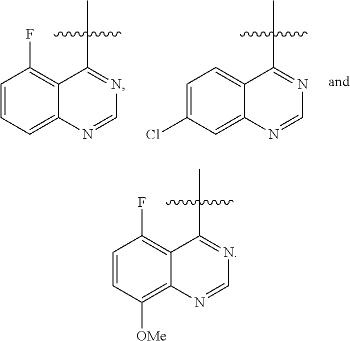

Clause 60. The method of any one of clauses 4-34, wherein Y is of the formula:

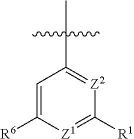

wherein:
Z¹ and Z² are each independently selected from CH and N;
R¹ is selected from H, alkyl, substituted alkyl, alkenyl, substituted alkenyl, heterocycle and substituted heterocycle;
R⁶ is selected from heterocycle, substituted heterocycle, cycloalkyl, substituted cycloalkyl, aryl and substituted aryl.
   Clause 61. The method of clause 60, of the formula:

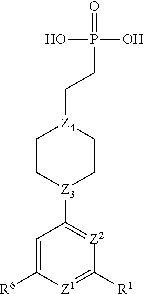

wherein: Z³ and Z⁴ are each independently selected from CR and N, wherein R is H, alkyl or substituted alkyl.
Clause 62. The method of clause 60 or 61, wherein Y is selected from:

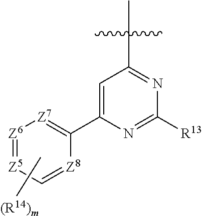

wherein,
Z⁵, Z⁶, Z⁷ and Z⁸ are each independently selected from CR¹⁴ and N;
R¹³ is selected from H, alkyl, substituted alkyl, alkenyl, substituted alkenyl, heterocycle and substituted heterocycle;
each R¹⁴ is independently selected from H, OH, alkyl, substituted alkyl, alkenyl, substituted alkenyl, alkoxy, substituted alkoxy, —OCF₃, amine, substituted amine, amide, heterocycle and substituted heterocycle; and
m is 0-5.
   Clause 63. The method of clause 60 or 61, wherein Y is selected from:

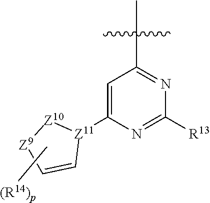

wherein,
Z⁹, Z¹⁰ and Z¹¹ are each independently selected from CR¹⁴ and N;
R¹³ is selected from H, alkyl, substituted alkyl, alkenyl, substituted alkenyl, heterocycle and substituted heterocycle;
each R¹⁴ is independently selected from H, OH, alkyl, substituted alkyl, alkenyl, substituted alkenyl, alkoxy, substituted alkoxy, —OCF₃, amine, substituted amine, amide, heterocycle and substituted heterocycle; and
p is 0-4.
   Clause 64. The method of any one of clauses 60-63, wherein Y is selected from:

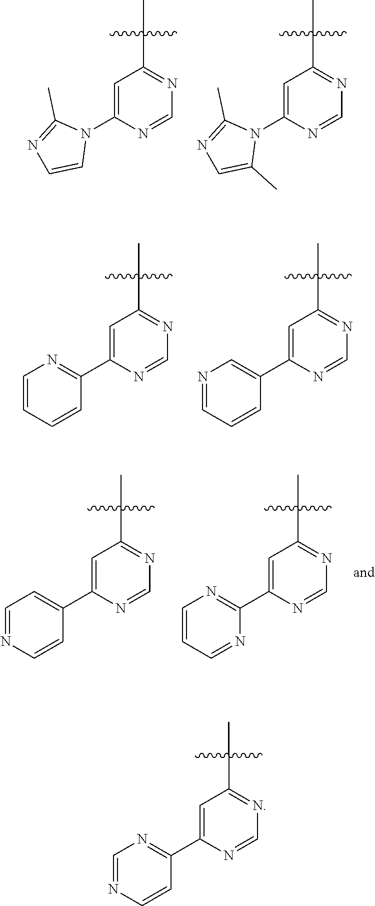

Clause 65. The method of any one of clauses 4-34, wherein Y is a group of the formula:

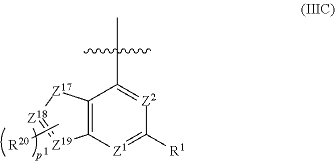

wherein,
Z¹, Z², Z¹⁷, Z¹⁸ and Z¹⁹ are each independently selected from CR²⁰ and N;
each R²⁰ is independently selected from H, OH, alkyl, substituted alkyl, alkenyl, substituted alkenyl, alkoxy, substituted alkoxy, —OCF₃, amine, substituted amine, amide, heterocycle and substituted heterocycle; and
p¹ is an integer from 0-4.
   Clause 66. The method of clause 65, wherein Y is of the structure:

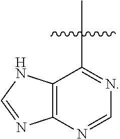 [SMILES: *c1ncnc2nc[nH]c12]

Clause 67. The method of any one of clauses 1-65, wherein the ENPP1 inhibitor is selected from the group consisting of

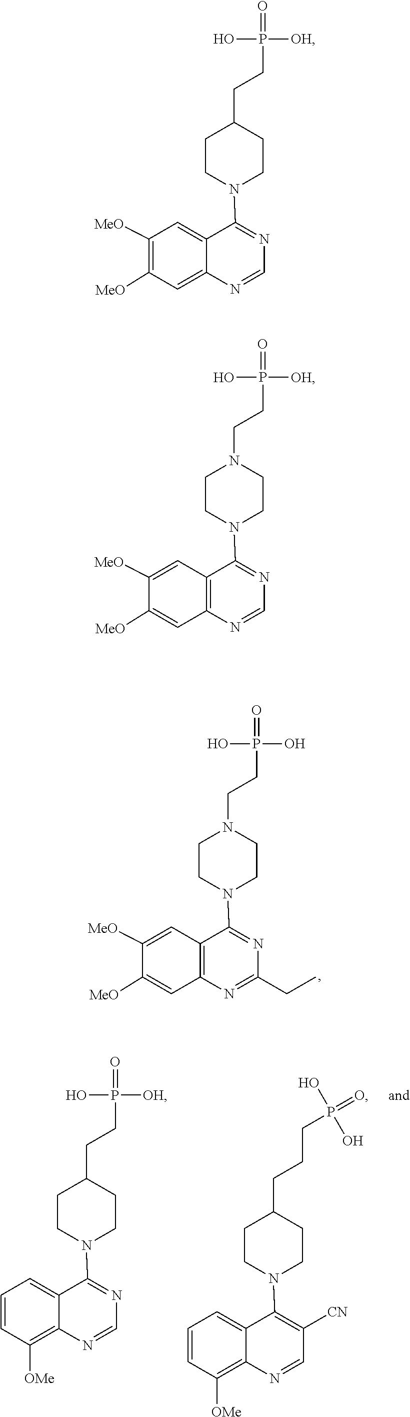 [SMILES: COc1cc2ncnc(N3CCC(CCP(=O)(O)O)CC3)c2cc1OC ; COc1cc2ncnc(N3CCN(CCP(=O)(O)O)CC3)c2cc1OC ; CCc1nc(N2CCN(CCP(=O)(O)O)CC2)c2cc(OC)c(OC)cc2n1 ; COc1cccc2c(N3CCC(CCP(=O)(O)O)CC3)ncnc12 ; COc1cccc2c(N3CCC(CCCP(=O)(O)O)CC3)c(C#N)cnc12]

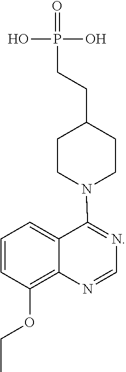 [SMILES: CCOc1cccc2c(N3CCC(CCP(=O)(O)O)CC3)ncnc12]

Clause 68. The method of any one of clauses 4-67, wherein the ENPP1 inhibitor is a compound selected from the compounds of Table 1 and Table 2.
Clause 69. The method of any one of claims 4-67, wherein the ENPP1 inhibitor is in a pharmaceutical composition, comprising:
a ENPP1 inhibitor of any one of clauses 4-67; and
a pharmaceutically acceptable excipient.
   Clause 70. The method of any one of clauses 1-69, wherein the cancer is a solid tumor cancer.
   Clause 71. The method of clause 70, wherein the cancer is a lymphoma.
   Clause 72. The method of clause 70 or 71, wherein the cancer is selected from, adrenal, liver, kidney, bladder, breast, colon, gastric, ovarian, cervical, uterine, esophageal, colorectal, prostate, pancreatic, lung (both small cell and non-small cell), thyroid, carcinomas, sarcomas, glioblastomas, melanoma and various head and neck tumors.
   Clause 73. The method of clause 72, wherein the cancer is breast cancer.
   Clause 74. The method of clause 72, wherein the cancer is glioblastoma.
   Clause 75. The method of any one of clauses 70-74, further comprising administration of one or more additional active agents.
   Clause 76. The method of clause 75, wherein the one or more additional active agents is a chemotherapeutic agent or an immunotherapeutic agent.
   Clause 77. The method of clause 75 or 76, wherein the one or more additional active agents is a small molecule, an antibody, an antibody fragment, an antibody-drug conjugate, an aptamer, or a protein.
   Clause 78. The method of anyone of claims 2-75, wherein the immune checkpoint inhibitor is selected from a cytotoxic T-lymphocyte-associated antigen 4 (CTLA-4) inhibitor, a programmed death 1 (PD-1) inhibitor and a PD-L1 inhibitor.
   Clause 79. The method of clause 78, wherein the immune checkpoint inhibitor is atezolizumab, avelumab, durvalumab, cemiplimab, ipilimumab, pembrolizumab, or nivolumab.
   Clause 80. The method of clause 79, wherein the immune check point inhibitor is ipilimumab, pembrolizumab, or nivolumab.
   Clause 81. The method of any one of clauses 75-77, wherein the one or more additional active agents comprises a chemotherapeutic agent.
   Clause 82. The method of clause 81, wherein the chemotherapeutic agent is a cGAMP-inducing chemotherapeutic.
   Clause 83. The method of clause 82 wherein cGAMP-inducing chemotherapeutic is an anti-mitotic or antineoplastic agent administered in an amount effective to induce the production of cGAMP in the subject.
   Clause 84. The method of clause 3, wherein the chemotherapeutic agent is a cytotoxic agent.
   Clause 85. The method of clause 3, wherein the chemotherapeutic agent is selected from the group consisting of daunorubicin, doxorubicin, epirubicin, idarubicin, mitoxantrone, and valrubicin.
   Clause 86. The method of clause 3, wherein the chemotherapeutic agent is doxorubicin.
   Clause 87. The method of any one of clauses 4-83, wherein the inhibitor is administered to the subject before radiation therapy.
   Clause 88. The method of any one of clauses 4-83, wherein the inhibitor is administered following exposure of the subject to radiation therapy.
   Clause 89. The method of any one of clauses 4-88, wherein the radiation therapy induces the production of cGAMP in the subject.
   Clause 90. The method of any one of clauses 4-89, wherein the radiation therapy is administered at a dosage and/or frequency effective to reduce radiation damage to the subject.
   Clause 91. The method of any one of clauses 1-90, wherein the ENPP1 inhibitor is cell impermeable.
   Clause 92. The method of any one of clauses 1-90, wherein the ENPP1 inhibitor is cell permeable.
   Clause 93. The method of any one of claims 1-92, wherein the subject is to undergo or has undergone resection of a malignant tumor.
   Clause 94. The method of any one of clauses 1-93, wherein the method comprises suppressing or preventing the recurrence of the cancer.
   Clause 95. The method of any one of clauses 1-94, wherein the method comprises suppressing or preventing malignant tumor cells from colonizing or invading vascular tissue.

1. A method of treating, reducing, or preventing metastasis of cancer in a subject in need thereof, comprising administering to the subject a therapeutically effective amount of a composition comprising an ectonucleotide pyrophosphatase/phosphodiesterase 1 (ENPP1) inhibitor.

2. The method of claim 1, further comprising administering to the subject a therapeutically effective amount of a composition comprising an immune checkpoint inhibitor in combination with the composition comprising an ENPP1 inhibitor.

3. The method of claim 1, further comprising administering to the subject a therapeutically effective amount of a composition comprising a chemotherapeutic agent in combination with the composition comprising an ENPP1 inhibitor.

4. The method of claim 1, further comprising administering radiation therapy to the subject.

5. The method of claim 1, wherein the ENPP1 inhibitor comprises the formula (VI): 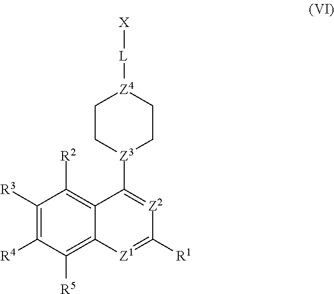
wherein,
X is a hydrophilic head group selected from boronic acid, hydroxamic acid, phosphonic acid, phosphonate, phosphonate ester, phosphate, phosphate ester, thiophosphate, thiophosphate ester, phosphoramidate and thiophosphoramidate;
L is a linker;
Z¹ and Z² are each independently selected from CR¹ and N;
Z³ and Z⁴ are each independently selected from CR and N, wherein R is H, alkyl or substituted alkyl;
each R¹ is independently selected from H, cyano, trifluoromethyl, halogen, amino, alkyl, substituted alkyl, alkenyl, substituted alkenyl, heterocycle and substituted heterocycle;
R² and R⁵ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle;
R³ and R⁴ are each independently selected from H, OH, alkyl, substituted alkyl, alkenyl, substituted alkenyl, alkoxy, substituted alkoxy, —OCF₃, halogen, amine, substituted amine, amide, heterocycle and substituted heterocycle; or R³ and R⁴ together with the carbon atoms to which they are attached form a fused ring selected from heterocycle, substituted heterocycle, cycloalkyl, substituted cycloalkyl, aryl and substituted aryl;
or a pharmaceutically acceptable salt or a solvate thereof.

6. The method of claim 5, wherein:
L is selected from —CH₂—, —(CH₂)₂—, —(CH₂)₃—, —(CH₂)₄—, —(CH₂)₅— and —(CH₂)₆—;
X is selected from: 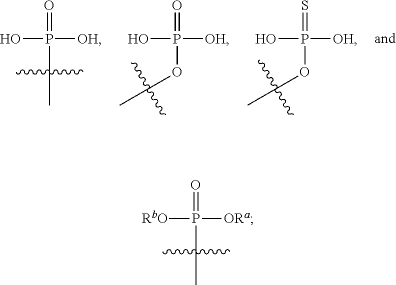
wherein:
R^{a} and R^{b} are each independently selected from aryl, alkyl, —CH₂OC(O)R^{e}, and —CH₂OC(O)OR^{e}; and
wherein R^{e} is alkyl.

7. The method of claim 6, wherein the ENPP1 inhibitor is of the formula: 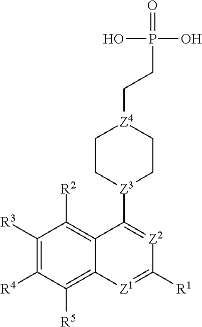
wherein,
Z¹ and Z² are each N;
Z³ is N; and
Z⁴ is CH or N.

8. The method of claim 5, wherein the portion of the ENPP1 inhibitor represented by 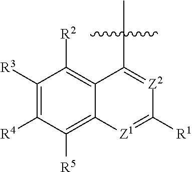
is selected from:

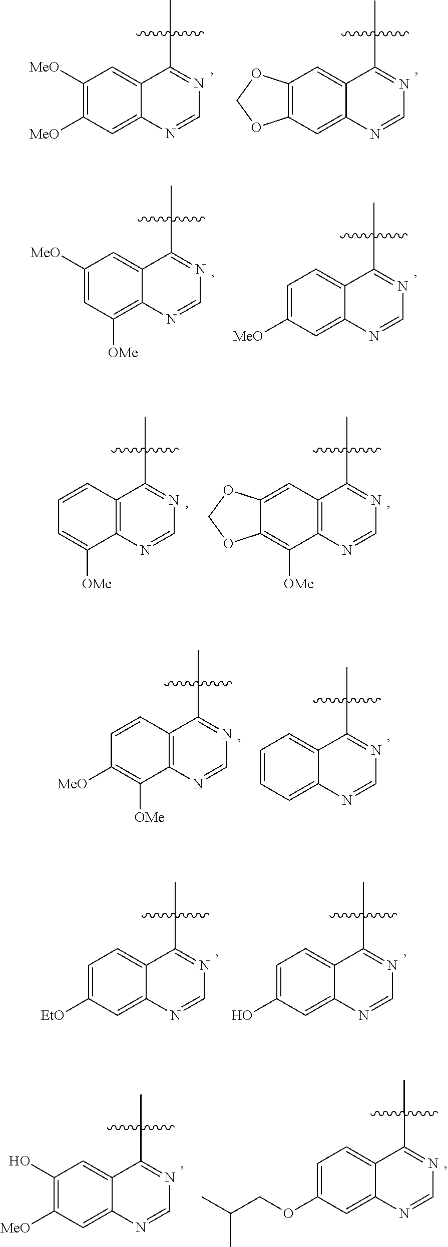

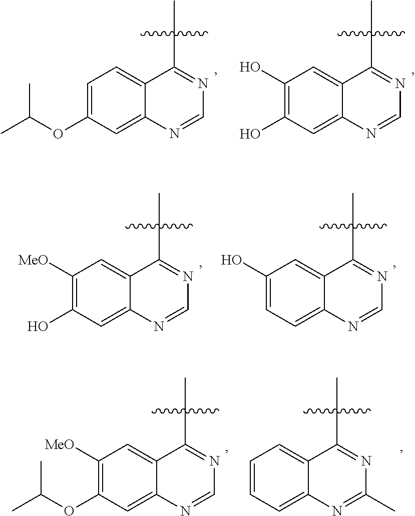

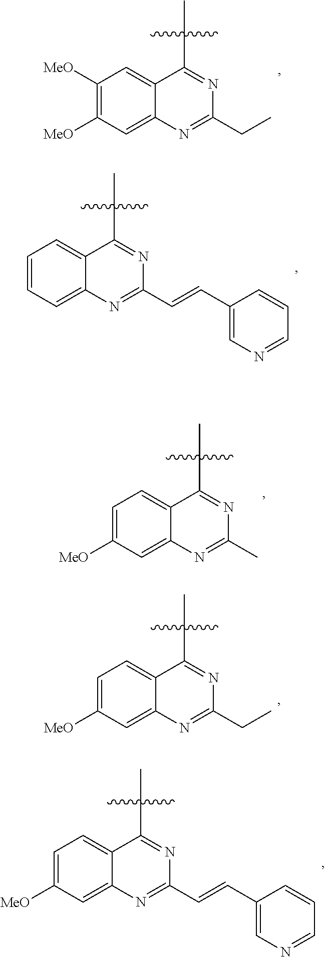 [SMILES: *c1nc(CC)nc2cc(OC)c(OC)cc12 ; *c1nc(/C=C/c2cccnc2)nc2ccccc12 ; *c1nc(C)nc2cc(OC)ccc12 ; *c1nc(CC)nc2cc(OC)ccc12 ; *c1nc(/C=C/c2cccnc2)nc2cc(OC)ccc12]

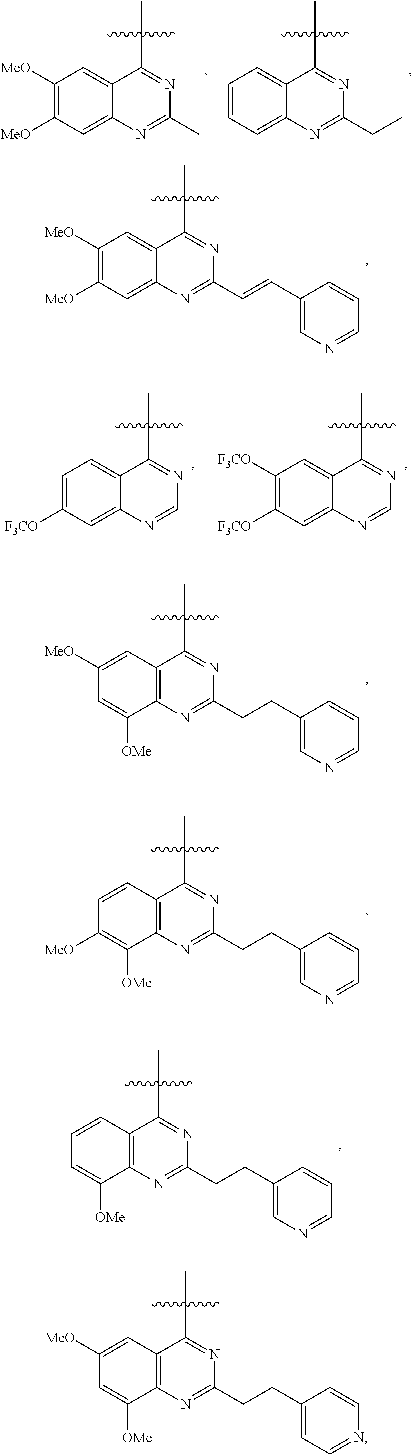

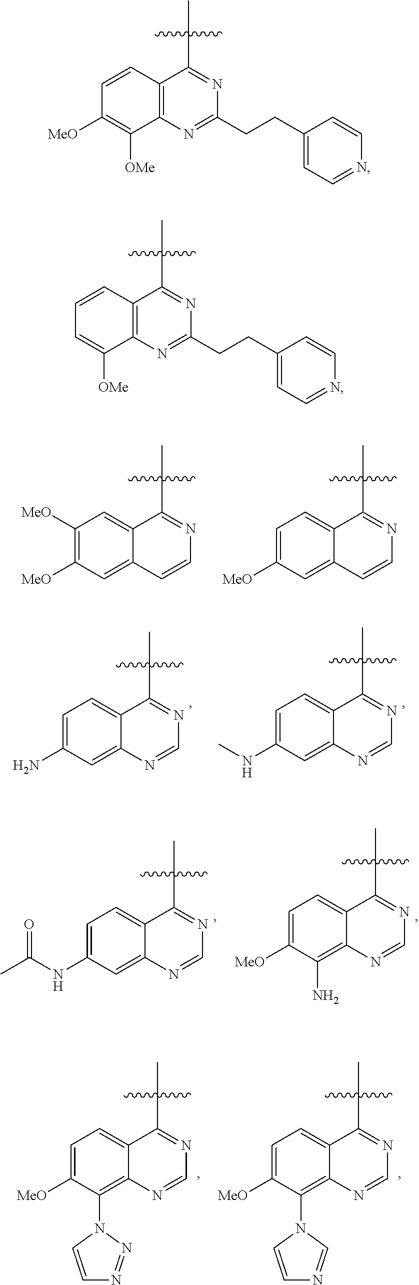

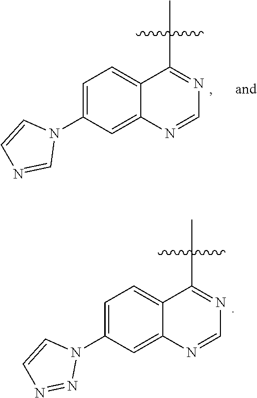 [SMILES: *c1ncnc2cc(-n3ccnc3)ccc12 ; *c1ncnc2cc(-n3ccnn3)ccc12]

9. The method of claim 5, wherein the ENPP1 inhibitor is selected from the group consisting of

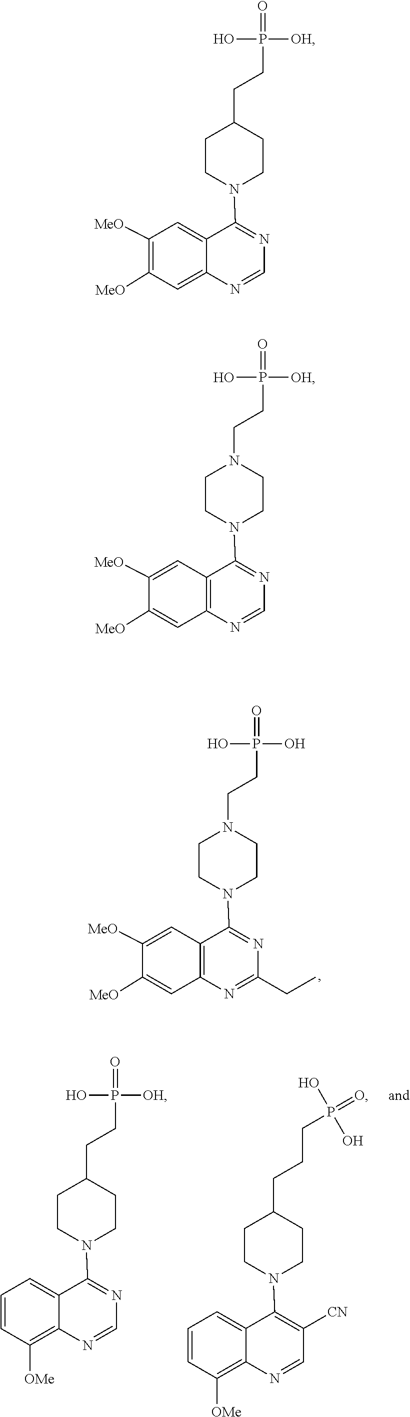 [SMILES: COc1cc2ncnc(N3CCC(CCP(=O)(O)O)CC3)c2cc1OC ; COc1cc2ncnc(N3CCN(CCP(=O)(O)O)CC3)c2cc1OC ; CCc1nc(N2CCN(CCP(=O)(O)O)CC2)c2cc(OC)c(OC)cc2n1 ; COc1cccc2c(N3CCC(CCP(=O)(O)O)CC3)ncnc12 ; COc1cccc2c(N3CCC(CCCP(=O)(O)O)CC3)c(C#N)cnc12]

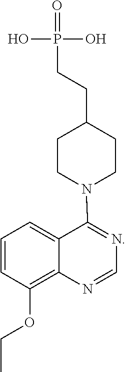 [SMILES: CCOc1cccc2c(N3CCC(CCP(=O)(O)O)CC3)ncnc12]

10. (canceled)

11. The method of claim 2, wherein the immune checkpoint inhibitor is a cytotoxic T-lymphocyte-associated antigen 4 (CTLA-4) inhibitor, a programmed death 1 (PD-1) inhibitor, or a PD-L1 inhibitor.

12. The method of claim 11, wherein the immune checkpoint inhibitor is atezolizumab, avelumab, durvalumab, cemiplimab, ipilimumab, pembrolizumab, or nivolumab.

13. (canceled)

14. The method of claim 3, wherein the chemotherapeutic agent is a cytotoxic agent.

15. The method of claim 3, wherein the chemotherapeutic agent is selected from the group consisting of daunorubicin, doxorubicin, epirubicin, idarubicin, mitoxantrone, and valrubicin.

16. The method of claim 15, wherein the chemotherapeutic agent is doxorubicin.

17. The method of claim 1, wherein the subject is to undergo or has undergone resection of a malignant tumor.

18. The method of claim 1, wherein the method comprises suppressing or preventing the recurrence of the cancer.

19. The method of claim 1, wherein the method comprises suppressing or preventing malignant tumor cells from colonizing or invading vascular tissue.

20. The method of claim 1, further comprising administering to the subject radiation therapy and a therapeutically effective amount of a composition comprising an immune checkpoint inhibitor in combination with the composition comprising an ENPP1 inhibitor.

21. The method of claim 20, wherein the immune checkpoint inhibitor is a cytotoxic T-lymphocyte-associated antigen 4 (CTLA-4) inhibitor, a programmed death 1 (PD-1) inhibitor, or a PD-L1 inhibitor.

22. The method of claim 20, wherein the immune checkpoint inhibitor is atezolizumab, avelumab, durvalumab, cemiplimab, ipilimumab, pembrolizumab, or nivolumab.
